(19) United States
(12) Patent Application Publication
(10) Pub. No.: US 2010/0227794 A1
(43) Pub. Date: Sep. 9, 2010

(54) SMART CONTRAST AGENT AND METHOD FOR DETECTING TRANSITION METAL IONS AND TREATING RELATED DISORDERS

(75) Inventors: Eric Martin Yezdimer, State College, PA (US); Tomohiro Umemoto, Kusatsu (JP)

Correspondence Address: STEIN MCEWEN, LLP, 1400 EYE STREET, NW, SUITE 300, WASHINGTON, DC, 20005, US

(73) Assignee: I.S.T. Corporation, Shiga (JP)

(21) Appl. No.: 12/730,536

(22) Filed: Mar. 24, 2010

Related U.S. Application Data
(63) Continuation-in-part of application No. 12/324,258, filed on Nov. 26, 2008.

(51) Int. Cl. A61K 38/00; C07K 2/00; A61P 25/28; A61P 25/16; A61P 25/18; A61P 25/24; A61P 35/00

(52) U.S. Cl. 514/2; 530/300

(57) ABSTRACT

The present disclosure provides smart contrast agents for transition metals and a method of using the same. The smart contrast agents include a core peptide and a first labeling group attached to a first end of the core peptide. The smart contrast agents can also include a second labeling group attached to a second end of the core peptide. The core peptide can bind to transition metals, and can be homologous to a fragment selected from the extended octarepeat region of a prion protein.

### CROSS-REFERENCE TO RELATED APPLICATION

This application claims the benefit of U.S. patent application Ser. No. 12/324,258, filed Nov. 26, 2008, the disclosure of which is incorporated herein, by reference.

This application incorporates by reference the sequence listing in the ASCII text file entitled “Replacement Sequence Listing,” created on Apr. 19, 2010, having a size of 10 KB.

### BACKGROUND

1. Field

Aspects of the present invention relate to a smart molecular contrast agent, and a method of using the smart contrast agent to detect transition metals and treat abnormal transition metal pathologies.

2. Description of the Related Art

Abnormal distributions of transition metals inside the body are potential diagnostic and predictive markers for several central nervous system diseases, including Alzheimer's disease, Parkinson's disease, bipolar disorders, depression, prion diseases, and glioblastomas. A key occurrence of many of these diseases is a disturbance of the natural homeostasis of transition metals inside the body. Methods that can non-invasively image transition metal distributions inside the body would be profoundly useful in diagnosing, studying, and treating a wide range of medical disorders.

The distribution of transition metals inside the human body is not uniform, and concentrations vary widely depending on the tissue or fluid being examined. For example, thin slice laser ablation inductively coupled plasma mass spectrometry (LA-ICP-MS) studies of human (M V Zorly, M Dehnhardt, G Reifenberger, K Zilles and J S Becker Imaging of Cu, Zn, Pb and U in human brain tumor resections by laser ablation inductively coupled plasma mass spectrometry. International Journal of Mass Spectrometry. 2006; 257: 27-33. J S Becker, M V Zorly, C Pickhardt, N Palomero-Gallagher and K Zilles Imaging of copper, zinc, and other elements in thin section of human brain samples (hippocampus) by laser ablation inductively coupled plasma mass spectrometry. Analytical Chemistry. 2005; 77: 3208-16) and rodent (B Jackson, S Harper, L Smith and J. Flinn. Elemental mapping and quantitative analysis of Cu, Zn, and Fe in rat brain sections by laser ablation ICP-MS. Analytical and Bioanalytical Chemistry. 2006; 384: 951-57) brain tissues reveal that the concentrations of zinc and copper are significantly localized on the millimeter scale, in various subsections of brain anatomy. Millimeter imaging resolutions have become common place when using magnetic resonance imaging (MRI) techniques, but direct in vivo imaging of key transition metals remains a challenge. The reason is that local variations of transition metal ions are often insufficient to impart enough signal variation for direct detection using current MRI techniques. To overcome this challenge, smart molecular contrast agents can be used. Smart contrast agents are molecules that undergo a chemical response to the presence of a target molecule or atom in their localized environment. Smart contrast agents are constructed in such a way that their chemical response to the target species induces a detectable modification of the electronic signals measured during an imaging scan. This modification varies as a function of the target species concentration. Smart fluorescence molecular contrast agents to detect transition metals are available, but in general, non-invasive fluorescence imaging is greatly limited in its application. Some smart MRI molecular contrast agents for transition metal imaging are also available, but currently have several important limiting characteristics. Therefore, there is a need for a new and improved series of compounds that can act as smart molecular contrast agents, for medical imaging of transition metals.

The use of MRI methods, to non-invasively examine and map the human body, has been one of the great advances of modern medicine. In particular, the ability to use MRI to diagnose a wide range of medical conditions has led to great improvements in the quality and overall success of patient care. Many variants of MRI technology have been reported.

During a typical MRI procedure, a primary static magnetic field, ranging between 0.1 to 3.0 Tesla, is applied to a patient. The longitudinal axis of the magnetic field is typically applied in the head to foot direction and labeled as the Z-axis. The primary magnetic field aligns the atoms with half-integer nuclear spins in one of two possible directions. The first direction is parallel to the magnetic field vector (B₀), and the second direction is antiparallel to the magnetic field vector. The parallel direction is the low energy state and the antiparallel direction represents a slightly higher energy state. The nuclear spins will also precess, in an out of phase manner, about the Z-axis. The frequency of the precession, otherwise called the Larmor frequency, is determined by the gyromagnetic ratio of the atomic nucleus, multiplied by the primary magnetic field strength. For example, the gyromagnetic ratio of ¹H is 42.6 MHz/Tesla, meaning that a B₀ strength of 1.5 Tesla will cause the nuclear spins of ¹H atoms to precess, at a frequency of 63.9 MHz.

In the presence of the primary magnetic field, the majority of the spin vectors inside a tissue sample cancel each other out, because of the occurrence of similar populations of both the low and high energy spin states. Boltzmann statistics, however, dictate that a small imbalance in the populations will exist, with the lower energy state having a slightly larger population. This imbalance of atomic nuclear spin states produces a small net magnetization of the patient's tissues, along the Z-axis. No net magnetization is present in the X-Y plane, because of the canceling effect caused by the spins precessing out of phase with each other.

The targeted atomic nucleus of choice of most MRI methods is ¹H. This is because of the natural high abundance of ¹H in water and biological tissues. Once the atomic spins reach equilibrium, after exposure to the primary static magnet, the diamagnetic tissues will exhibit a weak magnetic field along their longitudinal axis. This field is then perturbed by a radio frequency (RF) pulse that corresponds to the Larmor frequency of the targeted atomic nuclei. The RF pulse causes some of the targeted atomic spins to precess in phase with each other and produces a net magnetic field in the X-Y plane. Some of the atomic spins are also promoted to higher energy spin states and cause the spin vectors to tip away from the longitudinal axis, thereby reducing the net magnetic field of the tissue in the Z direction.

After the RF pulse has subsided, the atomic nuclei in the magnetized tissue sample undergo relaxation processes and return to their unperturbed alignment and out of phase precession about the Z-axis of the primary magnetic field. The field from the magnetized tissue induces a current the in RF coil, as described by Faraday's law of induction, and the energy imparted to the atomic spins from the RF pulse is dissipated. This is known as free induction decay (FID) and can be detected electronically in the RF coil.

The spin relaxation rate back to its unperturbed Z-axis alignment is called the longitudinal relaxation rate (T₁) and describes the recovery of tissue magnetization along the Z-axis. The rate of relaxation in the X-Y plane is referred to as the spin-spin, or transverse relaxation rate (T₂), and is governed by spin dephasing caused by the exchange of energy among the precessing atomic spins. The decay rate of the T₁ and T₂ relaxations occurs in an exponential fashion. The value of T₁ has been defined as the time required for the net magnetization along the Z-axis to recover (1-exp⁻¹), or 63.2% of its unperturbed value. The value of T₂ has been defined as the amount of time required for the magnetization in the X-Y plane to decay (1-exp⁻¹), or 63.2% of its perturbed value. The values for T₁ and T₂ are dependent on the chemical composition of tissue being sampled. For example the T₁ values at a B₀ field strength of 0.1 Tesla for liver, spleen, muscle, and fatty tissues are 265.0±47.4 ms, 394.6±17.3 ms, 309.8±48.5 ms, 152.5±11.9 ms, respectively. The T₁ times for water are considerable higher at 2000-3000 ms (J Halavaara, S Lukkarinen, R Sepponen, A Markkola and J. Tanttu. Contrast-to-noise ratio of multiple slice spin lock technique: prospects for liver imaging. British Journal of Radiology. 2003; 76: 788-91).

Gradient magnets are secondary magnets located inside the primary magnetic field. The purpose of the gradient magnets is to encode the signals received by the RF coil, so as to include information describing their individual spatial origins. They generate much weaker and more precise magnetic fields than the primary magnet. Under the influence of the primary magnet, all the atomic spins are aligned in a homogenous nature (along the Z axis), and the currents induced in the RF coil are therefore dependent on all of the atomic spins, regardless of their location inside the body.

The gradient magnets apply weak, but precisely controlled, linearly increasing magnetic fields, in X, Y, and Z directions. This has the effect of introducing a gradient in the net magnetic field, for example, by slightly increasing the magnetic field in the positive Z direction (i.e., head) and slightly decreasing the magnetic field in the negative Z direction (i.e., feet). The linear variation in the magnetic field strength across the body causes the precessional frequency of the atomic spins to vary linearly across the body. By controlling the exact frequency of the RF pulse to coincide with the Larmor frequency at a given position, it is possible to perturb only those atomic spins that are located along a designated magnetic field line. This limits the FID signals to those emanating from a precise two dimensional cross section of the body (otherwise known as a Z-slice).

The exact position of the atomic spins within the Z-slice can be encoded with X and Y position information, by applying additional gradient magnetic fields in those directions, after the RF pulse has ended. Applying a gradient magnetic field in the Y direction causes the precession frequency of atomic spins to be altered slightly; causing the atomic spins located in the upper Y direction to begin precessing out of phase with the atomic spins located in the lower Y direction. After a set amount of time, the gradient magnetic field in the Y direction is turned off. The atomic spins then return to their original uniform pre-Y magnet precession frequency, but the amount of dephasing that occurred during the time the magnet was energized, remains. The third gradient magnet is then turned on along the X direction, again causing the atomic spin frequencies to be altered linearly along the X direction.

The measurement of the FID yields a frequency spectrum that is tied to the position the atomic spins along the X direction. This procedure is then repeated many times, each time incrementally increasing the duration of the gradient magnetic field along the Y direction, in order to increase the amount of dephasing present in the atomic spins, before the FID frequency spectrum from the X-axis is recorded. This allows a two dimensional frequency versus phase, K-space map, of the FID signals to be produced. Two dimensional Fourier transformations then allow a spatial domain image of the Z-slice to be obtained. The entire process is then repeated for different cross sectional Z-slices, until an entire three dimensional image has been generated. Because the images are based on localized T₁ and T₂ relaxations times, which vary with tissue compositions, it is possible to distinguish between different tissue types in the image, often with millimeter resolutions.

Often it is desirable to further increase the relaxation rate of blood during a MRI procedure. Tumors, vascular leaks, and scar tissues often exhibit abnormal blood accumulation or pooling, but are sometimes difficult to distinguish from healthy tissues in an MRI image. To improve the contrast between healthy and unhealthy tissues, a common procedure is to inject a highly soluble paramagnetic compound into the bloodstream. Paramagnetic compounds used for this purpose are called MRI contrast agents (W Krause editor. Contrast Agents I: Magnetic Resonance Imaging. Berlin; Springer-Verlag, 2002). The MRI contrast agent increases the amount of electron-proton coupling, leading to faster energy dissipation from the atomic spins excited by the RF pulse. This decreases the time required for the atomic spins to return to their original state and promotes a change in the received signal that is a function of the in vivo distribution of the MRI contrast agent.

The paramagnetic compounds are usually small organic molecules tightly bound to a lanthanide metal ion. Several lanthanides, such as gadolinium, have favorable paramagnetic properties, but are toxic if administered into the body in their free ion form. Strong complexation with an organic chelater significantly reduces the toxicity. The change produced in T₁ relaxation times, due to the addition an MRI contrast agent, is often characterized by the following equation:

$${T}_{1 , obs} = {( \frac{1}{{T}_{1 , d}} + {n}_{L} {r}_{1 , p} [ L ] )}^{- 1} \qquad ( Equation 1 )$$

where T_{1,obs} is the observed longitudinal relaxation rate, T_{1,d} is the relaxation from the diamagnetic compounds in the sample, r_{1,p} is the relaxivity of a contrast agent measured in units of 1/(mM s), [L] is the concentration of paramagnetic ion introduced, and n_{L} is the number of paramagnetic ions contained in the MRI contrast agent. MRI contrast agents that are highly soluble in blood are sometimes referred to as blood-pool agents and are useful in identifying areas of the body with subtle variations in blood accumulation. Several blood-pool contrast agents are currently available, and a non-exhaustive list of these compounds, in their uncomplexed form, is as follows:
1,4,7,10-tetraazacyclododecane-1,4,7,10-tetraacetic acid [commercial name: Dotarem®] (DOTA) (AD Sherry, inventor The Board of Regents, The University of Texas System, assignee. Gadolinium chelates as NMR contrast agents. U.S. Pat. No. 4,639,365. 1984),
1,4,7,10-tetraazacyclododecane-1,4,7-trisacetic acid (DO3A),
10-(2-hydroxypropyl)-1,4,7,10-tetraazacyclododecane-1,4,7-triacetic acid [commercial name ProHance®] (HP-DO3A) (M F Tweedle, G T Gaughan and J T Hagan, inventor E. R. Squibb & Sons, Inc., assignee. 1-substituted-1,4,7-triscarboxymethyl-1,4,7,10-tetraazacyclododecane and analogs. U.S. Pat. No. 4,885,363. 1989),
10-[(2,3-dihydroxy-1-hydroxymethylpropyl]-1,4,7,10-tetraazacyclododecane-1,4,7-triacetic acid [commercial name: Gadovist®] (DO3A-B) (A Berg and T Almen, inventor Nycomed Imaging AS, assignee. Aminopolycarboxylic acids and derivatives thereof from magnetic resonance imaging. U.S. Pat. No. 5,419,893. 1995),
Diethylenetriamine-N,N,N′,N″,N″-pentaacetic acid [commercial name: Magnevist®] (DTPA) (H Gries and H-J Weinmann, inventor Schering Aktiengesellschaft, assignee. Diagnostic media. U.S. Pat. No. 4,647,447. 1987. H Gries, D Rosenberg, H-J Weinmann, U Speck, W Muetzel, G-A Hoyer and H Pfeiffer, inventor Schering Aktiengesellschaft, assignee. Sterile pharmaceutical compositions of gadolinium chelates useful enhancing NMR imaging. U.S. Pat. No. 4,957,939. 1990. H Gries, H-J Weinmann, U Speck, W Mutzel, G-A Hoyer and H Pfeiffer, inventor Schering Aktiengesellschaft, assignee. Method to enhance NMR imaging using chelated paramagnetic ions. U.S. Pat. No. 4,963,344. 1990), and
diethylenetriamine pentaacetic acid bismethylamide [commercial name: Omniscan®] (DTPA-BMA).

Agents with large n_{L}r_{1,p} values are preferred, because they increase the relaxation rate to a larger extent, enable faster MRI scanning times, and increase the ability to detect smaller variations in blood concentrations. This helps medical professionals to diagnose smaller abnormities in the patient's vascular system, which are often a sign of cancer, stroke, or injury.

Metallofullerenes and texaphyrins are examples of second generation MRI contrast agents with significantly higher relaxivities than traditional DOTA-based and DTPA-based compounds. Metallofullerenes are typically composed of a lanthanide atom trapped inside a carbon nanosphere cage (D K MacFarland, K L Walker, R P Lenk, S R Wilson, K Kumar, C L Kepley and J R Garbow. Hydrochalarones: A novel endohedral metallofullerene platform for enhancing magnetic resonance imaging contrast. Journal of Medicinal Chemistry. 2008; 51: 3681-83. R D Bolskar, A F Benedetto, L O Husebo, R E Prive, E F Jackson, S Wallace, L J Wilson and J M Alford. First soluble M@C derivates provide enhanced access to metallofullerenes and permit in vivo evaluation of Gd@C[C(COOH)] as a MRI contrast agent. Journal of the American Chemical Society. 2003; 125: 5471-78. R D Bolskar and J M Alford, inventor assignee. Derivatization and solubilization of fullerenes for use in therapeutic and diagnostic applications. U.S. patent application Ser. No. 10/263,375. 2003. M B Miller, K A Murphy, H C Doren, S A Stevenson, J P Stevenson and S Luo, inventor Luna Innovation, Inc., assignee. Imaging and treatment method for body. U.S. Pat. No. 6,471,942. 2002. M Mikawa, H Kato, M Okumura, M Narazaki, Y Kanazawa, N Miwa and H Shinohara. Paramagnetic water-soluble metallofullerences having the highest relaxivity for MRI contrast agents. Bioconjugate Chemistry. 2001; 12: 510-14), Texaphyrins are composed of an expanded porphyrin ring that can complex with a lanthanide atom, most notably gadolinium or lutetium. Some texaphyrin-lanthanide complexes also have potential therapeutic applications as radiation enhancers and photosensitizers (J L Sessler and R A Miller. Texaphyrins: New drugs with diverse clinical applications in radiation and photodynamic therapy. Biochemical Pharmacology. 2000; 59: 733-39). Several classes of metal nanoparticles may also be used as MRI contrast agents including iron oxide particles, gold nanoparticles, and gold nanoshells (SP Leary, C Y Liu and M L J Apuzzo. Toward the emergence of nanoneurosurgery: Part II: Nanomedicine: Diagnostics and imaging at the nanoscale level. Neurosurgery. 2006; 58: 805-23). Besides possessing a significant ¹H relaxivity, gold nanoparticles and gold nanoshells can also be designed to serve as therapeutic photosensitizers (D P O'Neal, L R Hirsch, N J Halas, J D Payne and J L West. Photo-thermal tumor ablation in mice using near infrared-absorbing nanoparticles. Cancer Letters. 2004; 209: 171-76).

Other atomic nuclei, such as ¹⁹F, have also been used in MRI applications. The gyromagnetic ratio for ¹⁹F is 40.1 MHz/Tesla. Therefore, ¹⁹F precesses at a different frequency than ¹H atoms exposed to the same primary magnetic field. The use of ¹⁹F MRI methods can offer advantages of over ¹H MRI methods, for several applications. The primary advantage is that ¹⁹F MRI images offer greatly reduced background signals, because of the very low natural occurrence of ¹⁹F inside the body. One exemplary application of note is the positional tracking, inside the body, of injected compounds, cells, or other materials that have artificially labeled ¹⁹F atoms.

Another class of MRI imaging, that differs from the traditional relaxivity based approach described above, uses chemical exchange saturation transfer (CEST) to produce non-invasive images (K M Ward, A H Aletras and R S Balaban. A new class of contrast agents for MRI based on proton chemical exchange dependent saturation transfer (CEST). Journal of Magnetic Resonance. 2000; 143: 79-87. A D Sherry and M Woods. Chemical exchange saturation transfer contrast agents for magnetic resonance imaging. Annual Review of Biomedical Engineering. 2008; 10: 391-411). During CEST imaging an RF pulse is directed at the protons of a contrast agent. The energy of RF pulse promotes spin flipping processes that eventually yield a saturated spin state with an equal population of parallel and anti-parallel ¹H nuclear spins within the contrast agent. Chemical exchange processes, usually between amines, amides, hydroxyls and the surrounding water solvent occur on timescales faster than nuclear spin relaxation. As the saturated spins from the contrast agent are exchanged with other molecules in the surrounding molecular environment, the net magnetization of the main population of non-contrast agent ¹H atoms is decreased. The amount of magnetization loss can be measured and used to produce a CEST imagine.

The presence of paramagnetic ions, capable of inducing hyperfine shifts, in the contrast agent increases the chemical shift of endogenous protons and permits better selection of the resonance frequency of the contrast agent protons by the RF pulse. The use of a paramagnetic ion in a CEST procedure is often referred to as PARACEST. This is particularly important during in vivo imaging, because the resonance spectrum of the hydrogen atoms is broaden by their complex biological environment.

In traditional relaxation MRI methods gadolinium(III) is often used as the paramagnetic ion. Gadolinium(III) is special case in the lanthanide series because it has seven unpaired electrons distributed isotropically in its 4 f orbitals. This prevents gadolinium(III) from inducing hyperfine nuclear magnetic resonance shifts in neighboring protons, thus, contrast agents using gadolinium(III) to not alter the Larmor frequency of surrounding protons. This property is advantageous for relaxivity based imaging, but is not appropriate PARACEST imaging. Other lanthanides ions do however exhibit hyperfine shifts. Dysprosium is a lanthanide that does produce hyperfine shifts but has a weak effect on T₁ and T₂ relaxation times. Fast nuclear spin relaxation times are not desirable in PARACEST imaging, because they lessen the amount of time the exchanged saturated spins have before they re-equilibrate.

Another method capable of non-invasive imaging is called positron emission tomography (PET). PET imaging works by detecting gamma ray electromagnetic radiation emitted during the nuclear decay process of unstable tracer radioisotopes. The tracer radioisotopes (H Jadvar and J A Parker. Clinical PET and PET/CT. New York: Springer, 2005. S Dresel editor. PET in Oncology. New York: Springer, 2008), which require a cyclotron to produce, are incorporated inside biologically active or tagged molecules, and are often referred to as PET contrast agents. PET contrast agents are usually prepared on site, due to the prohibitively short half lives of the tracer radioisotopes (Table 1) and are injected into the patient immediately before conducting a PET scan.

TABLE 1
Selected PET and SPECT isotopes

| Isotope | Half-life | Decay Mode | γ-Energy (keV) | Application |
|---|---|---|---|---|
| ¹¹C | 20.4 minutes | β⁺ | 511 | PET |
| ¹³N | 9.97 minutes | β⁺ | 511 | PET |
| ¹⁵O | 122 seconds | β⁺ | 511 | PET |
| ¹⁸F | 110 minutes | β⁺ | 511 | PET |
| ^{99m}Tc | 6 hours | γ | 140 | SPECT |
| ¹¹¹In | 72 hours | γ | 250 | SPECT |
| ¹²³I | 13 hours | γ | 160 | SPECT |

The unstable tracer radioisotopes used in PET scans are neutron-deficient and undergo a decay process where a proton is transmuted into a neutron, positron (β⁺), and an electron neutrino (ν). The positron travels a few millimeters before it encounters an electron and annihilates it, in a matter-antimatter reaction. The particle annihilation produces gamma ray photons that propagate in opposite linear directions, from the annihilation point. A PET machine is comprised of thousands of discrete scintillator crystals that surround the patient in a series of linked rings. Each crystal is attached to a photomultiplier tube that is in turn connected to a computer. The gamma ray photons interact with the crystals, causing a burst of longer wavelength light that is detected by the photomultiplier tubes. The path that the gamma rays take is called the line of response (LOR). For each annihilation event, it is expected that crystals on opposite sides of the patient should each register a gamma ray, at approximately the same time.

By comparing the crystals' position in the detection ring and the time of each impact, it is possible to calculate the LOR for a single radioisotope decay event. Unpaired gamma rays that are detected are considered noise and are discarded. The radioisotope position should be located somewhere along the LOR. After many LOR have been recorded, a three dimensional map overlaying all the LOR can be constructed. Areas with higher concentrations of tracer radioisotopes will have more LORs running through them, and hence, a stronger density on the PET image.

The most common of PET contrast agent is fluorodeoxyglucose (FDG), where the fluorine component is comprised of the relatively short-lived ¹⁸F isotope. Increased muscle, tissue, or brain activity, often result in increased glucose usage, and thus, localized areas of increased glucose concentration can be mapped three dimensionally, using PET. Complexation of FDG with other biomolecules, such as antibodies targeting specific cellular receptors, or other compounds, is commonplace. This permits the mapping of areas with elevated concentrations of antigens and has been profoundly useful in detecting and/or confirming the presence of many types of ailments, including cancer, heart disease, and Alzheimer's disease.

Still another method of medical imaging is called single photon emission computed tomography (SPECT). SPECT is very similar to PET, however, a key difference is that the tracer radioisotope used directly emits gamma rays during its decay process, rather than requiring a positron-electron annihilation reaction. In SPECT, there is no dual emission of gamma ray photons, so the gamma rays are measured in a two dimension fashion, using a specially designed gamma ray camera. Multiple two dimension images are obtained from different vantage points of the subject, and a three dimensional model is constructed using well documented computation techniques.

The radioisotope tracers used in SPECT are therefore different from those used in PET studies. A common isotope used is the metastable ^{99m}Tc nucleus, which has a half-life of 6 hours and a gamma emission at 0.14 MeV. This half life is much longer than those typical to PET scans and is a significant advantage to the practical application of SPECT versus PET. In a similar fashion to MRI contrast agents, the ^{99m}Tc metal ion is often complexed with other organic chelater molecules, in order to lower its toxicity, and to allow the tracer to be chemically attached to various antibodies or other biomolecules.

A key property of methods like PET and SPECT is that the local physiological environment of the tracer isotope does not strongly affect the magnitude of signal received by the imager. Therefore, the signal intensity of PET and SPECT images is proportional to only the concentration of the contrast agent. Images from PET, x-ray computed tomography (CT), SPECT, and MRI scans can also be run in combination, to give composite, fused images.

Fluorescence imaging is another method available to investigate living cells and tissues in a non to semi-invasive manor. During fluorescence imaging, a fluorescence dye is injected into a tissue or animal model. This sample is then exposed to ultraviolet or visible light, to excite the nonbinding electrons of the dye into a higher energy state. The dye molecule then undergoes vibrational relaxations, while in the excited state, reducing the energy gap between the excited and ground states. Eventually a photon is released, when the excited electron is returned to the ground state. The lowering of the energy gap has the effect of increasing the wavelength (i.e., Stokes' shift) of the emitted photon.

The emission wavelength is usually in the visible range, although some fluorescence dyes have emission spectra that extend into the near-infrared and infrared ranges. Digital cameras and/or photomultiplier tubes record the emitted light. Wavelength filters, monochromators, or polychromators are often used to measure the exact wavelength of the light emitted and allow the creation of two dimensional images at multiple emission wavelengths. Numerous fluorescence dyes, designed for a variety or purposes, are commercially available.

The use of fluorescence imaging in vitro cell experiments, ex vivo tissue experiments, and in vivo rodent studies is commonplace, because of its low cost, ease of use, and decreased restrictions on the degree of surgical invasiveness of laboratory animals. Practical application of fluorescence imaging in humans has proven more difficult, because of the limited penetration of the excitation and emission light through human tissue. The problem is exasperated for imaging of brain centralized disorders, because of the presence of the encasing skull bone. However, these factors do not necessarily preclude the use of fluorescence imaging in humans, particularly during open surgery, or medical procedures relying on catheters and endoscopes.

Fluorescence dyes can be designed to act as smart molecular contrast agents. Fluorescence is an electron driven phenomenon and is strongly influenced by the electrostatic environment of the fluorescence dye. In this regard, fluorescence imaging is similar to MRI imaging, in that the contrast agents respond to their local environments. This is in contrast to PET and SPECT imaging, in which the gamma ray emission from the contrast agents is largely independent of their local environment.

A common method of constructing a smart fluorescence contrast agent is to employ phenomena called Förster resonance energy transfer (FRET) (L Stryer. Fluorescence energy transfer as a spectroscopic ruler. Annual Review of Biochemistry. 1978; 47: 819-46. R M Clegg. Fluorescence resonance energy transfer. Fluorescence imaging spectroscopy and microscopy, series. X F Wang and B Harman editor. John Wiley and Sons, Inc., 1996. C Berney and G Danuser. FRET or No FRET: A quantitative comparison. Biophysical Journal. 2003; 84: 3992-4010). The acronym FRET is also referred to as fluorescence resonance energy transfer. FRET is a process where an excited fluorescent dye (the donor) can non-radioactively transfer a portion of its energy to a neighboring molecule (the acceptor). The acceptor is often a fluorescence dye itself, although this is not required.

Electromagnetic energy absorbed by the donor molecule is transferred to the acceptor molecule, through a coupling of the electrostatic dipole-dipole interactions between the donor and acceptor molecules. The transfer of energy causes an electron in the acceptor molecule to become electronically excited, reducing the amount of fluorescence observed from the donor molecule. The acceptor molecule may also emit its own photon, after an internal vibrational relaxation phase, which is significantly red-shifted, when compared to the donor emission spectrum. The efficiency of the energy transfer depends on the orientation of the donor and acceptor molecules' transition dipole moments and the distance between the two groups. The maximal range of most FRET donor and acceptor pairs is usually less than 100 Angstroms. A common implementation of FRET probes is in the use of tracing protein-protein interactions in cellular microscopy, where one protein is tagged with a donor molecule, and a second protein is tagged with an acceptor molecule. The fluorescence image of the cell will then exhibit FRET effects, if the two proteins of interest are within 100 Angstroms of each other, indicating an in vitro co-localization of the two proteins. FRET methodologies have also been used to create smart fluorescence constant agents for pH, using labeled DNA chains (T Ohmichi, Y Kawamoto, P Wu, D Miyoshi, H Karimata and N Sugimoto. DNA-based biosensor for monitoring pH in vitro and in living cells. Biochemistry. 2005; 44: 7125-30) and calcium ions (A Miyawaki, J Llopis, R Heim, J M McCaffery, J A Adams, M Ikura and R Y Tsien. Fluorescent indicators for Ca+2 based on green fluorescent proteins and calmodulin. Nature. 1997; 388: 882-7), using short peptide sequences coupled to fluorescence proteins (R Y Tsien The green fluorescent protein. Annual Review of Biochemistry. 1998; 67: 509-44. N C Shaner, R E Campbell, P A Steinbach, B N G Giepmans, A E Palm and R Y Tsien. Improved monomeric red, organic and yellow fluorescent proteins derived from Discosoma sp. red fluorescent protein. Nature Biotechnology. 2004; 22: 1567-72).

One example of a useful application of the current invention would be the early prediction of Alzheimer's disease. Alzheimer's disease is a degenerative neurological disease affecting roughly 1 in 2 of people over 85 years of age. It characterized by the formation of amyloid β (Aβ) plaques and neurofibrillary tangles in brain tissues, which eventually lead to pronounced neuronal destruction, memory loss, brain atrophy, and death. The normal functioning and pathology of Alzheimer's disease involves several versions of amyloid β proteins, which can exist in soluble, fragmented, and/or aggregate forms. Several imaging technologies utilizing amyloid binding groups for Alzheimer's disease detection have been reported. Some approaches of note include:
N-methyl-[¹¹C]2-(4′-methylaminophenyl)-6-hydroxybenzothiazole (PIB), a radiolabled modified thioflavin-T compound (W E Klunk, J Chester A. Mathis and Y Wang, inventor University of Pittsburgh, assignee. Thioflavin derivatives for use in the antemortem diagnosis of Alzheimer's disease and in vivo imaging and prevention of amyloid deposition. U.S. Pat. No. 7,351,401. 2004. W E Klunk, H Engler, A Nordberg, Y Wang, G Blomqvist, D P Holt, M Bergstrom, I Savitcheva, G-f Huang, S Estrada, B Ausen, M L Debnath, J Barletta, J C Price, J Sandell, B J Lopresti, A Wall, P Koivisto, G Antoni, C A Mathis and B Langström. Imaging brain amyloid in Alzheimer's disease with Pittsburgh compound-B. Annals of Neurology. 2004; 55: 306-19. W E Klunk, J Chester A. Mathis and Y Wang, inventor University of Pittsburgh, assignee. Thioflavin derivates for use in antemortem diagnosis of Alzheimer's disease and in vivo imaging and prevention of amyloid deposition. U.S. Pat. No. 7,270,800. 2003) for use in PET scans;
(E,E)-1-fluoro-2,5-bis(3-hydroxycarbonyl-4-hydroxy)stytylbenzene (FSB) for use with ¹H and ¹⁹F MRI (M Higuchi, N Iwata, Y Matsuba, K Sato, K Sasamoto and TC Saido. ¹⁹F and ¹H MRI detection of amyloid beta plaques in vivo. Nature Neuroscience. 2005; 8: 527-33);
an Ge/DTPA-Aβ₁₋₄₀ compound (J F Poduslo, T M Wengenack, G L Curran, T Wisniewski, E M Sigurdsson, S I Macura, B J Borowski and C R J Jr. Molecular targeting of Alzheimer's amyloid plaques for contrast-enhanced magnetic resonance imaging. Neurobiology of Disease. 2002; 11: 315-29. T Wisniewski, D Turnbull, E M Sigurdsson and Y Z Wadghiri, inventor New York University, assignee. Detection of Alzheimer's amyloid by magnetic resonance imaging. U.S. Pat. No. 6,821,504. 2004) for use with ¹H MRI;
stilbenes labeled compounds with Ge/DTPA, Ge/DOTA and Gd⁺³/DO3A groups for use with ¹H MRI (M J Allen Delivery and activation of contrast agents for magnetic resonance imaging. thesis 2004; California Institute of Technology, Pasadena. M J Allen, S Fraser, R E Jacobs and T J Meade, inventor California Institute of Technology, assignee. Magnetic resonance imaging agents for in vivo labeling and detection of amyloid deposits. U.S. Pat. No. 7,029,655. 2001); and
amyloid binding moieties comprising of a benzothiazole derivative chemically bound to DTPA (X Huang, J L Kremsky and P L C SR., inventor The General Hospital Corporation, Prime Organics, Inc., assignee. Amyloid-binding, metal-chelating agents. U.S. patent application Ser. No. 11/096,919. 2005. X Huang, inventor. Amyloid-binding, metal-chelating agents. U.S. patent application Ser. No. 10/762,965. 2004) for use with ¹H MRI.

All of these methods are very promising, but unfortunately suffer from several drawbacks. For example, thioflavins and styrylbenzenes are known to have nM affinities for both Aβ_{[1-40]} and Aβ_{[1-42]} aggregates (H F Kung, C-W Lee, Z-P Zhuang, M-P Kung, C Hou and K Plössl. Novel stilbenes as probes for amyloid plaques. Journal of the American Chemical Society. 2001; 123: 12740-41) which have different pathological roles and are difficult to distinguish, without compounds containing antibody-level selectivity. Small molecule amyloid targeting motifs may also non-selectivity bind other hydrophobic and amyloid-like lipid regions inside body tissues. Non-selective tissue binding in white brain matter greatly reduces the ability for early, pre-symptomatic detection of plaque formation in a PIB PET image, because the signals from any initial amyloid plaque formation are obscured by the background signals from nonselective PIB binding. PET imaging methods using, for example PIB, also suffer from the very short half lives of tracer radioisotopes like ¹¹C and necessitate a high rate of blood brain barrier passage and rapid body clearance, to achieve strong signal/noise ratios and selective imaging of amyloid deposits.

An alternative route for the imaging of brain tissues from Alzheimer's disease patients relates to transition metal homeostasis and imbalances caused by Alzheimer disease pathology. Imaging of transition metal distributions in vivo can be advantageous over antibody based imaging techniques, because the concentrations of certain transition metals can exceed the concentration of antibody targets, such as cellular receptors, neurotransmitters, and proteins by orders of magnitude (A I Bush. Metals and neuroscience. Current Opinion in Chemical Biology. 2000; 4: 184-91). Senile plaques from Alzheimer's disease patients have been found to contain significant accumulations of iron, zinc, and copper (M A Lovell, J D Robertson, W J Teesdale, J L Campbell and W R Markesbery. Copper, iron and zinc in Alzheimer's disease senile plaques. Journal of Neurological Sciences. 1998; 158: 47-52). Zinc affinities for several β-amyloid fragments, full proteins, and aggregations have been measured to be in the low μM range (C Talmard, A Bouzan and P Faller. Zinc binding to amyloid-beta: Isothermal titration calorimetry and Zn competition experiments with zinc sensors. Biochemistry. 2007; 46: 13658-66). Micromolar concentrations of zinc ions (EC₅₀ of 120-140 μM) have been found to strongly increase the specific conversion of endogenous β-amyloid peptides into insoluble aggregates (A M Brown, D M Tummolo, K J Rhodes, J R Hofmann, J S Jacobsen and J Sonnenburg-Reines. Selective aggregation of endogenous beta-amyloid peptide and soluble amyloid precursor protein in cerebrospinal fluid by zinc. Journal of Neurochemistry. 1997; 69: 1204-12). Treatments with clioquinol, a selective copper and zinc chelater molecule, have shown significant inhibition of Aβ accumulation in transgenic mice (R A Cherny, C S Atwood, M E Xilinas, D N Gray, W D Jones, C A McLean, K J Barnham, I Volitakis, F W Fraser, Y-S Kim, X Huang, L E Goldstein, R D Moir, J T Lim, K Beyreuther, H Zheng, R E Tanzi, C L Masters and A I Bush. Treatment with a copper-zinc chelator markedly and rapidly inhibits β-amyloid accumulation in Alzheimer's disease transgenic mice. Neuron. 2001; 30: 665-76). Studies of human cerebrospinal fluid (CSF) report strong inverse correlations of Aβ_{[1-42]} levels and copper, zinc, iron, manganese, and chromium (D Strozyk, L J Launer, P A Adlard, R A Chemy, A Tsatanis, I Volitakis, K Blennow, H Petrovitch, L R White and A I Bush. Zinc and copper module Alzheimer ABeta levels in human cerebrospinal fluid. Neurobiology of Aging. 2007; online). The link between the formation of amyloid plaques and these metals provides a reasonable expectation that high resolution imaging of these ions in brain tissue could provide pre-symptomatic prediction of the onset of Alzheimer's disease.

Concentrations of iron in the brain are generally the highest of all transition metals. The highly unpaired electron configuration of Fe^{+2/+3} atoms, and their routine complexation with large proteins like ferritin and transferrin, makes direct MRI detection feasible, without the aid of signal enhancing contrast agents. Changes in iron concentrations of Alzheimer's patients has been detected using MRI, by measuring the field dependent R₂ increase (FDRI) in the transverse relaxation rate of brain tissues (G Bartzokis, D Sultzer, J Mintz, L E Holt, P Marx, C K Phelan and S R Marder. In vivo evaluation of brain iron in Alzheimer's disease and normal subjects using MRI. Biological Psychiatry. 1994; 35: 480-7). Others have theorized about using MRI to directly detect observed imbalances in manganese (II) ions in prion diseases (B-S Wong, S G Chen, M Colucci, Z Xie, T Pan, T Liu, R Li, P Gambetti, M-S Sy and D R Brown. Aberrant metal binding by prion protein in human prion disease. Journal of Neurochemistry. 2001; 78: 1400-08). In vivo detection of transition metals with fewer unpaired electrons, or a lower physiologically abundance, is not considered feasible without the use of selective smart contrast agents. Direct ¹H MRI detection of copper and zinc has not been achieved in vivo to date, because of a combination of lower concentrations and significantly weaker interactions with the surrounding ¹H nuclear spins.

### SUMMARY

Aspects of the present invention relate to a smart contrast agent for transition metals, and a method of using the same. Aspects of the present invention also relate to the use of the contrast agents to treat medical disorders exhibiting abnormal transition metal pathologies.

According to aspects of the present invention, provided is a smart contrast agent comprising: a core peptide comprising a transition metal binding domain derived from the prion protein (SEQ ID NOS: 1 and SEQ ID NOS: 2) that is constructed using 70% homologous fragments from the extended octarepeat region of SEQ ID NOS: 3; and a labeling group attached to the core peptide.

According to aspects of the present invention, provided is a smart contrast agent comprising: a core peptide comprising a transition metal binding domain derived from a prion protein (SEQ ID NOS: 1 and SEQ ID NOS: 2) that is constructed using 70% homologous fragments from the extended octarepeat region of SEQ ID NOS: 3; a first labeling group attached to a first end of the core peptide; and a second labeling group attached to a second end of the core peptide.

According to aspects of the present invention, provided is a method of detecting a transition metal, comprising: applying the smart contrast agent to a mammal; and detecting whether the smart contrast agent has bound to a transition metal.

According to aspects of the present invention, provided is a method of treating disease, comprising: delivering the smart contrast agent into a mammal; and altering the distribution of the transition metal inside the mammal, such that negative effects of a disease are reduced.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 illustrates a smart contrast agent including a first labeling group, according to aspects of the present invention;

FIG. 2 illustrates another smart contrast agent including first and second labeling groups, according to aspects of the present invention;

FIGS. 3A-3D illustrate smart contrast agents, according to aspects of the present invention;

FIGS. 4A-4D illustrate smart contrast agents, according to aspects of the present invention;

FIGS. 5A-5E illustrate smart contrast agents, according to aspects of the present invention;

FIG. 6 illustrates a smart contrast agent, according to aspects of the present invention;

FIG. 7 illustrates an electrospray mass spectrum of a smart contrast agent P1P, according to aspects of the present invention;

FIG. 8 illustrates a 600 MHz ¹H NMR spectrum of P1P, according to aspects of the present invention;

FIGS. 9A-9D illustrate the activity of 10 μM P1P when titrated with zinc(II), copper(II), magnesium(II), and calcium(II) ions, where the diamonds, boxes, and triangles denote pH conditions of 7.6, 7.0, and 6.4, respectively;

FIGS. 10A-10C illustrate the activity of 10 μM P1P mutants when titrated with copper(II) ions;

FIGS. 11A-11C illustrate the activity of 10 μM P1P mutants when titrated with for zinc(II) ions;

FIG. 12 illustrates the bovine serum stability of the P1P peptide, where column A, B, and C denote P1P exposed to serum for 0 hours, P1P exposed to serum for 2 hours, and P1P in a control phosphate buffer, respectively;

FIGS. 13A-13C illustrate the activity of 10 μM P1P mutants, when titrated with gadolinium(III) ions;

FIG. 14 illustrates MALDI mass spectroscopy results for a P15/Gd complex;

FIGS. 15A and 15B illustrate the HPLC of the P15/Gd complex, using a detection wavelength of 205 nm;

FIGS. 16A and 16B illustrate the HPLC of the P15/Gd complex, using a detection wavelength of 463 nm;

FIGS. 17A and 17B illustrate the activity of the P15/Gd complex, at concentrations of 10 and 100 μM, respectively;

FIGS. 18A-18C illustrate the reversibility of a 100 μM P15/Gd complex, when titrated with zinc(II), copper(II), and calcium(II) ions. FIG. 18C shows the effect of adding excess EDTA after the reactions described in FIG. 18A are obtained;

FIGS. 19A-19B illustrate ¹H MRI relaxivity of the P15/Gd complex in different copper and calcium solutions, respectively;

FIG. 20 illustrates an electrospray mass spectrum of a smart contrast agent P41, according to aspects of the present invention;

FIG. 21 illustrates a 600 MHz ¹H NMR spectrum of P41, according to aspects of the present invention;

FIG. 22A-22C illustrate the activity of 10 μM P41 when titrated with magnesium(II), calcium(II), manganese(II), iron(II), zinc(II), copper(II), gadolinium (III) ions, at pH=7.6, pH=7.0 and pH=6.4;

FIG. 23A illustrates the bovine serum stability of the P41 and P42 peptides;

FIG. 23B illustrates the bovine serum stability of the P45 and P46 peptides;

FIG. 23C illustrates the bovine serum stability of the P58 and P48 peptides;

FIG. 23D illustrates the bovine serum stability of the P57 and P57/Gd peptides;

FIG. 23E illustrates the bovine serum stability of the P1S and P1P peptides;

FIG. 23F illustrates the bovine serum stability of the P31 peptide;

FIG. 23G illustrates the bovine serum stability of the P43, P44, and P50 peptides;

FIG. 24 illustrates an electrospray mass spectrum of a smart contrast agent P50, according to aspects of the present invention;

FIG. 25 illustrates a 600 MHz ¹H NMR spectrum of P50, according to aspects of the present invention;

FIG. 26A-26C illustrate the activity of 10 μM P50 when titrated with magnesium(II), calcium(II), manganese(II), iron(II), zinc(II), copper(II), gadolinium (III) ions at pH=7.6, pH=7.0 and pH=6.4;

FIGS. 27A-27B illustrate fluorescence images of the approximate position of P50 inside a nude mouse, using respective excitation pulses of 430 and 460 nm;

FIG. 28 illustrates an electrospray mass spectrum of a smart contrast agent P20, according to aspects of the present invention;

FIG. 29 illustrates a 600 MHz ¹H NMR spectrum of P20, according to aspects of the present invention;

FIGS. 30A-30D illustrate the activity of 10 μM P20 when titrated with zinc(II), copper(II), magnesium(II), and calcium(II) ions, where the diamonds, boxes, and triangles denote pH conditions of 7.6, 7.0, and 6.4, respectively;

FIG. 31A illustrates an electrospray mass spectrum of a smart contrast agent P57, according to aspects of the present invention;

FIG. 31B illustrates an electrospray mass spectrum of a smart contrast agent P57/Gd, according to aspects of the present invention;

FIG. 32 illustrates a 600 MHz ¹H NMR spectrum of P57, according to aspects of the present invention;

FIG. 33A-33C illustrate the activity of 10 μM P57/Gd when titrated with magnesium(II), calcium(II), manganese(II), iron(II), zinc(II), copper(II), gadolinium (III) ions at pH=7.6, pH=7.0 and pH=6.4;

FIG. 34 illustrates the ¹H MRI relaxivity of the P57/Gd complex in different copper solutions;

FIG. 35 illustrates the in vitro imaging of P57/Gd. Wells numbered 1-5 and 6-10 represent increasing concentrations of P57/Gd, ran in duplicate. The concentrations displayed are 0 μM, 10 μM, 25 μM, 50 μM, and 100 μM of P57/Gd;

FIG. 36A illustrates a coronal slice from an ¹H MRI image of an ICR mouse;

FIG. 36B illustrates several images from a SPECT scan of an ICR mouse;

FIG. 37 illustrates an electrospray mass spectrum of a smart contrast agent P54, according to aspects of the present invention;

FIG. 38 illustrates a 600 MHz ¹H NMR spectrum of P54, according to aspects of the present invention;

FIG. 39A-39B illustrate the activity of 10 μM P54 and P58 when titrated with, calcium(II), zinc(II), and copper(II) ions at pH=7.6, respectively; and

FIG. 40A-40B illustrate the absorbance spectrum of P54 and P58, respectively, where the solid curve denotes the contrast agent only and the dashed curve denotes the contrast agent in the presence of 3 equivalents of copper(II) ions at pH=7.6.

### DETAILED DESCRIPTION OF THE EXEMPLARY EMBODIMENTS

Reference will now be made in detail to the exemplary embodiments of the present invention, examples of which are illustrated in the accompanying drawings. The exemplary embodiments are described below, in order to explain aspects of the present invention, by referring to the figures.

“Homology”, “homologs of”, “homologous”, “identity”, or “similarity” refers to sequence similarity between two polypeptides, with identity being a more strict comparison. Homology and identity can each be determined by comparing a position in each sequence that may be aligned for purposes of comparison. When a position in the compared sequence is occupied by the same amino acid, then the molecules are identical at that position. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences.

The normal, healthy conformation of the cellular prion protein (PrP^{C}) is a short-lived glycoprotein, and is found anchored to the outside membranes of a wide range of cell types. The expression of the prion is exceptionally high in brain tissue, and it is thought to play a regulatory role in the oxidation chemistry of the brain. Several debilitating neurodegenerative diseases, including scrapie in sheep and goats, bovine spongiform encephalopathy, Creutzfeldt-Jakob disease, Gerstmann-Stäussler-Scheinker syndrome, fatal familial insomnia, and kuru, are characterized by the production of a misfolded conformation of an endogenous cellular form of the prion protein. The misfolded “scrapie” prions (PrP^{Sc}) form partially protease-resistant aggregates, eventually leading to the formation of plaques and neuron death. Metal imbalances of copper and manganese have been found in the brains of scrapie infected mice, prior to the onset of symptoms (A M Thackray, R Knight, S J Haswell, R Bujdoso and D R Brown. Metal imbalance and compromised antioxidant function are early changes in prion disease. Biochemistry Journal. 2002; 362: 253-58). Similar metal imbalances have been found in human brains with sporadic Creutzfeldt-Jakob disease (B-S Wong, S G Chen, M Colucci, Z Xie, T Pan, T Liu, R Li, P Gambetti, M-S Sy and DR Brown. Aberrant metal binding by prion protein in human prion disease. Journal of Neurochemistry. 2001; 78: 1400-08).

The entire PrP^{C} is capable of binding 4-5 Cu⁺² ions and has been theorized to act as natural sensor and transport mechanism for extracellular copper ions (A P Garnett and J H Viles. Copper binding to the octarepeats of the prion protein. Journal of Biological Chemistry. 2003; 278: 6795-802). One area of prion research has focused on a conserved region with a repeated sequence of eight amino acids PrP^{C}_{[60-91]}, as the primary source of metal biochemistry, and is located near the non-membrane bound N-terminus of the protein. Table 2 shows the correspondence between the mouse and human prion sequences.

TABLE 2
Alignment of the Mouse and Human Prion Sequence

| | |
|---|---|
| Human 1 | Met Ala Asn Leu Gly Cys Trp Met Leu Val |
| Mouse 1 | Met Ala Asn Leu Gly Tyr Trp Leu Leu Ala |
| | *** *** *** |
| Human 11 | Leu Phe Val Ala Thr Trp Ser Asp Leu Gly |
| Mouse 11 | Leu Phe Val Thr Met Trp Thr Asp Val Gly |
| | *** *** *** *** |
| Human 21 | Leu Cys Lys Lys Arg Pro Lys Pro Gly Gly |
| Mouse 21 | Leu Cys Lys Lys Arg Pro Lys Pro Gly Gly |
| Human 31 | Trp Asn Thr Gly Gly Ser Arg Tyr Pro Gly |
| Mouse 31 | Trp Asn Thr Gly Gly Ser Arg Tyr Pro Gly |
| Human 41 | Gln Gly Ser Pro Gly Gly Asn Arg Tyr Pro |
| Mouse 41 | Gln Gly Ser Pro Gly Gly Asn Arg Tyr Pro |
| Human 51 | Pro Gln Gly Gly Gly Gly Trp Gly Gln Pro |
| Mouse 51 | Pro Gln Gly Gly --- Thr Trp Gly Gln Pro |
| | *** *** |
| Human 61 | His Gly Gly Gly Trp Gly Gln Pro His Gly |
| Mouse 60 | His Gly Gly Gly Trp Gly Gln Pro His Gly |
| Human 71 | Gly Gly Trp Gly Gln Pro His Gly Gly Gly |
| Mouse 70 | Gly Ser Trp Gly Gln Pro His Gly Gly Ser |
| | *** *** |
| Human 81 | Trp Gly Gln Pro His Gly Gly Gly Trp Gly |
| Mouse 80 | Trp Gly Gln Pro His Gly Gly Gly Trp Gly |
| Human 91 | Gln Gly Gly Gly Thr His Ser Gln Trp Asn |
| Mouse 90 | Gln Gly Gly Gly Thr His Asn Gln Trp Asn |
| | *** |
| Human 101 | Lys Pro Ser Lys Pro Lys Thr Asn Met Lys |
| Mouse 100 | Lys Pro Ser Lys Pro Lys Thr Asn Leu Lys |
| | *** |
| Human 111 | His Met Ala Gly Ala Ala Ala Ala Gly Ala |
| Mouse 110 | His Val Ala Gly Ala Ala Ala Ala Gly Ala |
| Human 121 | Val Val Gly Gly Leu Gly Gly Tyr Met Leu |
| Mouse 120 | Val Val Gly Gly Leu Gly Gly Tyr Met Leu |
| Human 131 | Gly Ser Ala Met Ser Arg Pro Ile Ile His |
| Mouse 130 | Gly Ser Ala Met Ser Arg Pro Met Ile His |
| | *** |
| Human 141 | Phe Gly Ser Asp Tyr Glu Asp Arg Tyr Tyr |
| Mouse 140 | Phe Gly Asn Asp Trp Glu Asp Arg Tyr Tyr |
| | *** *** |
| Human 151 | Arg Glu Asn Met His Arg Tyr Pro Asn Gln |
| Mouse 150 | Arg Glu Asn Met Tyr Arg Tyr Pro Asn Gln |
| | *** |
| Human 161 | Val Tyr Tyr Arg Pro Met Asp Glu Tyr Ser |
| Mouse 160 | Val Tyr Tyr Arg Pro Val Asp Gln Tyr Ser |
| | *** *** |
| Human 171 | Asn Gln Asn Asn Phe Val His Asp Cys Val |
| Mouse 170 | Asn Gln Asn Asn Phe Val His Asp Cys Val |
| Human 181 | Asn Ile Thr Ile Lys Gln His Thr Val Thr |
| Mouse 180 | Asn Ile Thr Ile Lys Gln His Thr Val Thr |
| Human 191 | Thr Thr Thr Lys Gly Glu Asn Phe Thr Glu |
| Mouse 190 | Thr Thr Thr Lys Gly Glu Asn Phe Thr Glu |
| Human 201 | Thr Asp Val Lys Met Met Glu Arg Val Val |
| Mouse 200 | Thr Asp Val Lys Met Met Glu Arg Val Val |
| Human 211 | Glu Gln Met Cys Ile Thr Gln Tyr Glu Arg |
| Mouse 210 | Glu Gln Met Cys Val Thr Gln Tyr Gln Lys |
| | *** *** *** |
| Human 221 | Glu Ser Gln Ala Tyr Tyr --- --- Gln Arg |
| Mouse 220 | Glu Ser Gln Ala Tyr Tyr Asp Gly Arg Arg |
| | *** *** *** |
| Human 229 | Gly Ser Ser Met Val Leu Phe Ser Ser Pro |
| Mouse 230 | Ser Ser Ser Thr Val Leu Phe Ser Ser Pro |
| | *** *** |
| Human 239 | Pro Val Ile Leu Leu Ile Ser Phe Leu Ile |
| Mouse 240 | Pro Val Ile Leu Leu Ile Ser Phe Leu Ile |
| Human 249 | Phe Leu Ile Val Gly |
| Mouse 250 | Phe Leu Ile Val Gly |

The underlined region in the above sequences is commonly referred to as the octarepeat region and consists of four repeats of the sequence PHGGGWGQ, hereafter referred to as the octarepeat unit. An expanded octarepeat region containing residues 51-100 in humans, or residues 51-99 in mouse, contains similar peptide sequences both upstream and downstream of the octarepeat region and is also biologically relevant to prion biometal chemistry. The full octarepeat region is capable of reducing Cu⁺² to Cu⁺, through a mechanism that is partially catalyzed by its tryptophan residues (F H Ruiz, E Silva and N C Inestrosa. The N-terminal tandem repeat region of human prion protein reduces copper: Role of tryptophan residues. Biochemical and Biophysical Research Communications. 2000; 269: 491-95).

Gene knockout experiments in mice lines have shown that completely removing the octarepeat sequences from the prion increases scrapie incubation times and profoundly modifies the presentation of the disease (E Flechsig, D Shmerling, I Hegyi, A J Raeber, M Fischer, A Cozzio, Cv Mering, A Aguzzi and C Weissman. Prion protein devoid of the octapeptide repeat region restores susceptibility to Scrapie in PrP knockout mice. Neuron. 2000; 27: 399-408). The octarepeat region and copper/zinc ions were found to be critical components in the rapid endocytosis of a biotinylated murine PrP (W Sumudhu, S Perera and N M Hooper. Ablation of the metal ion-induced endocytosis of the prion protein by disease-associated mutation of the octarepeat region. Current Biology. 2001; 11: 519-23). Genetic mutations producing additional inserts of octarepeat sequences are however associated with transmissible familial variants of Creutzfeldt-Jakob disease and Gerstmann-Stäussler-Scheinker syndrome although their role in the disease pathology remains unresolved (S Krasemann, I Zerr, T Weber, S Poser, H Kretzschmar, G Hunsmann and W Bodemer. Prion disease associated with a novel nine octarepeat repeat insertion in the PRNP gene. Brain Research. Molecular Brain Research. 1995; 34: 173-76. L G Goldfarb, P Brown, W R McCombie, D Goldgaber, G D Swergold and D C Gajdusek. Transmissible familial Creutzfeldt-Jakob disease associated with five, seven and eight extra octapeptide coding repeats in the PRNP gene. PNAS. 1991; 88: 10926-30).

Other studies have shown that the addition of copper ions cause PrP^{C} to undergo a rapid, albeit reversible, conversion to a protease-resistant form that is structurally distinct from PrP^{Sc}, as determined from antibody binding (E Quaglio, R Chiesa and D A Harris. Copper converts the cellular prion protein into a protease-resistant species that is distinct from the scrapie isoform. Journal of Biological Chemistry. 2001; 276: 11432-38). In transgenic mice, however, the octarepeat domain was found to be unnecessary for the conversion of a “mini-prion” adaptation of PrP^{C} into an infectious conformation, indicating that metal interactions with the normal expressed form of the octarepeat region are not responsible for the transmissibility or pathogenesis of prion disease (S Supattapone, P Bosque, T Muramoto, H Wille, C Aagaard, D Peretz, H-OB Nguyen, C Heinrich, M Torchia, J Safar, F E Cohen, S J DeAmond, S B Prusiner and M Scott. Prion protein of 106 residues creates an artificial transmission barrier for prion replication in transgenic mice. Cell. 1999; 96: 869-79. M Rogers, F Yehiely, M Scott and S B Prusiner. Conversion of truncated and elongated prion proteins into the scrapie isoform in cultured cells. PNAS. 1993; 90: 3182-86).

The crystal and solution structure of copper(II) bound to the HGGGW motif indicates that the Cu⁺² is square-pyramidal, coordinated with 3 nitrogens and 1 oxygen equatorial ligands and one axial water molecule (C S Burns, E Aronoff-Spencer, C M Dunham, P Lario, N I Avdievich, W E Antholine, M M Olmstead, A Vrielink, G J Gerfen, J Peisach, W G Scott and G L Millhauser. Molecular features of the copper binding sites in the octarepeat domain of the prion protein. Biochemistry. 2002; 41: 3991-4001. M Mentler, A Weiss, K Grantner, Pd Pino, D Deluca, S Fiori, C Renner, W M Klaucke, L Moroder, U Bertsch, H A Kretzschmar, P Ta van and F G Parak. A new method to determine the structure of the metal environment in metalloproteins: investigation of the prion protein octapeptide repeat Cu(2+) complex. European Biophysics Journal. 2005; 34: 97-112). Electron paramagnetic resonance (EPR) spectra of the Cu⁺²/HGGGW motif are indistinguishable from larger multiple octarepeats peptides (E Aronoff-Spencer, C S Burns, N I Avdievich, G J Gerfen, J Peisach, W E Antholine, H L Ball, F E Cohen, S B Prusiner and G L Millhauser. Identification of the Cu2+ binding sties in the N-terminal domain of the prion protein by EPR and CD spectroscopy. Biochemistry. 2000; 39: 13760-71) at physiological pH and imply the HGGGW motif may represent a generalized building block of the complete octarepeat region. The binding of successive copper atoms to the octarepeat region, however, yields an increasing affinity from the μM to fM scales and indicates the presence of cooperative interactions between the copper binding sites (A P Garnett, C E Jones and J H Viles. A survey of diamagnetic probes for copper²⁺ binding to the prion protein. ¹H NMR solution structure of the palladium²⁺ bound single octarepeat. Dalton Transactions. 2006; 509-18).

Using ¹H NMR, circular dichroism spectrometry and other experiments, other researchers (J H Viles, F E Cohen, S B Prusiner, D B Goodin, P E Wright and H J Dyson. Copper binding to the prion protein: Structural implications of four identical cooperative binding sites. PNAS. 1999; 96: 2042-47) hypothesized that a peptide composed of 2 octarepeat units could coordinate with copper in a square planar fashion, through the imidazole nitrogen of both histidine resides, a nitrogen ligand from the backbone of neighboring glycine reside, and a water molecule. They also proposed a similar cross octarepeat coordination for a 4 octarepeat peptide comprised of the sequence PrP^{C}_{[58-91]}. This is contrary to the self contained Cu⁺²/HGGGW building block hypothesis, but does offer an explanation for the source of the observed cooperative chemistry of prion copper binding. Other researchers have also found that the solution structure of the octarepeat peptides to be highly dependent on the number of octarepeat units, their concentration, pH and the complexing ion concentrations (D Valensin, M Luczkowski, F M Mancini, A Legowska, E Gaggelli, G Valensin, K Rolka and H Kozlowski. The dimeric and tetrameric octarepeat fragments of prion protein behave differently to its monomeric unit. Dalton Transactions. 2004; 1284-93). Other transition metals, including zinc, for example, are known to bind the octarepeat region. Binding affinities, using direct and glycine competitive assays with the PrP^{C}_{[52-98]} fragment has found the metal K_{d} to be 8 fM, 15 nM, 410 nM, and 2.95 mM for Cu⁺², Ni⁺², Zn⁺², and Mn⁺², respectively (G S Jackson, I Murray, L L P Hosszu, N Gibbs, J P Waltho, A R Clarke and J. Collinge. Location and properties of metal-binding sites on the human prion protein. PNAS. 2001; 98: 8531-35). Endocytosis of the prion protein has been observed for Cu⁺² and Zn⁺², but not Mn⁺² and is consistent with the weaker affinities for Mn⁺². The prion protein has also been theorized to play a critical role in neuronal zinc homeostatis (W Sumudhu, S Perera and N M Hooper. Ablation of the metal ion-induced endocytosis of the prion protein by disease-associated mutation of the octarepeat region. Current Biology. 2001; 11: 519-23).

Studies exploring the solution structure for octarepeat peptides binding with non-copper metal ions are limited. The binding structure of palladium (II) and nickel (II), to the peptide PrP^{C}_{[58-66]} (QPHGGGWGQ), were examined using ¹H NMR (A P Garnett, C E Jones and J H Viles A survey of diamagnetic probes for copper²⁺ binding to the prion protein. ¹H NMR solution structure of the palladium²⁺ bound single octarepeat. Dalton Transactions. 2006; 509-18). The NMR coordination geometry using Pd⁺² was found to be different from the crystal structure of the copper(II)/HGGGW complex. Although the peptide to metal stoichiometry remained the same at 1:1, the Cu⁺² interaction with the N_{δ} side chain of the histidine group was observed to convert to an interaction with the histidine N_{ε} atom. Palladium (II) also bound with the backbone nitrogens of the Gly_{[62]} and Gly_{[63]} residues, instead of the expected copper binding mode to Gly_{[61]} and Gly_{[62]}. The difference is believed to be due in part to the increased size of the Pd⁺² ion, and the inability of the peptide to form a tight coordinate ring structure, as in the case of the crystallized copper ion. Even more surprising, complexes with nickel (II) were paramagnetic, high spin species, implying a tetrahedral or octahedral binding geometry.

The general conclusion is that the octarepeat region of the prion can be selective to transition metal ions, whose proper homeostasis is critical for maintaining a healthy individual. Multiple complexation structures are thermally accessible at physiological conditions and the frequency of their occurrence depends on several variables, including: the type and concentrations of metals present, pH, and the exact subsection of the octarepeat region being examined.

The imaging of transition metal distributions, using a smart contrast agent that includes a peptide fragment(s) selected from the extended octarepeat region, is advantageous over previously reported small molecule, smart contrast agents. Estimates of the maximum concentration of copper and zinc ions in brain tissue range up to 500 μM, or perhaps higher. These metals however rarely exist in their free aqua form and are usually found strongly bound to various functional proteins. For example, it is estimated that 90% of the total concentration of zinc atoms is static and is locked up inside critical proteins and enzymes. Only the remaining 10% of the zinc is liable and potentially accessible to contrast agents. A similar situation exists for copper. The binding affinities for any smart contrast agent should be strong enough to compete with enough indigenous proteins in order to undergo a detectable amount of complexation with the targeted metal ions, but not strong enough to adversely interfere with the natural metal homeostasis in the body. The prion protein is highly conserved among mammals and normally found in significant concentrations inside the intercellular spaces of tissues. The octarepeat region has been found to possibly regulate copper and zinc concentrations, via endocytosis. Therefore it is reasonable to assume the binding affinities and hydrophobic/hydrophilic properties of the octarepeat region have already been largely optimized by evolutional processes for in vivo transition metal detection. Gene knock out experiments with mice have shown that viable mice are possible even with a complete lack of prion protein expression, so it is also reasonable to assume that temporary dosing with fragments from the extended octarepeat region should not be overly toxic.

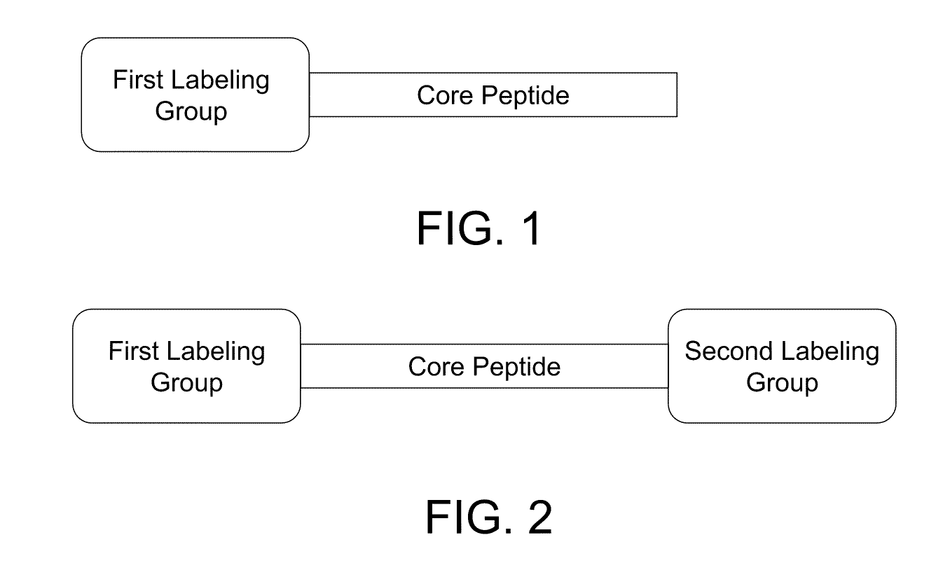

FIG. 1 illustrates a smart contrast agent, according to an exemplary embodiment of the present invention. As shown in FIG. 1, the smart contrast agent includes a core peptide and a first labeling group attached to an end of the core peptide. The core is a peptide sequence corresponding to selected fragment(s) from the extended octarepeat region of a prion protein of SEQ ID NOS 3. The extended octarepeat region is used as a primary template for the core peptide, because it is naturally expressed in the intercellular spaces of mammalian tissues. The core peptide can have metal affinities that will not cause a large interference with normal tissue transition metal homeostasis, but are strong enough to compete with other metalloproteins.

The core peptide can include a transition metal binding domain. Some embodiments of the core peptide have at least 70% homology with a peptide selected from SEQ ID NOS 4-22.

According to some embodiments, the core peptide can include multiple occurrences of peptides with at least 70% homology to SEQ ID NOS 4-22. For example, increasing the number of peptide occurrences from 1 to 2 can increase the binding affinities of the core peptide, because of cooperative effects between each peptide unit. For safety considerations the number of unmodified octarepeat units included in the core peptide would generally not exceed the number of normally expressed units, because genetic mutations producing PrP^{c} with additional inserts of octarepeat sequences have been associated with transmissible familial forms of Creutzfeldt-Jakob disease and Gerstmann-Stäussler-Scheinker syndrome. However, it is unlikely that a core peptide containing greater than 4 unmodified octarepeat units would make an effective sensor for Cu⁺², because of the sequence's documented ability to reduce Cu⁺² to Cu⁺. Other limiting factors, including low solubility and high molecular weight, are also expected to adversely affect the utility of smart contrast agents having a core peptide with greater than 4 unmodified octarepeat units.

The core peptide can include additional amino acids, in order to facilitate metal binding or the connection of one or more labeling groups to the core peptide. As referred to herein, a labeling group can be any compound that enhances the detectability of the core peptide. Examples of the labeling group include a fluorescent compound, a fluorescence quenching compound, a ¹H MRI labeling compound, a ¹⁹F MRI labeling compound, a PET labeling compound, a SPECT labeling compound, or a combination thereof.

Examples of the fluorescent compound include lissamine rhodamine B, naphtho fluorescein, rhodamine 6G, rhodamine red-X, tetramethylrhodamine, Texas red dye, X-rhodamine, eosin, erythrosin, fluorescein, HEX, JOE, Oregon green 488, Oregon green 514, rhodamine green, 2′,4′,5′,7′-tetrabromosulfonefluorescein, TET, BODIPY, cascade yellow, dansyl, dapoxyl, NBD, PyMPO, pyrene, and the like. The fluorescent compound can be, for example, a fluorophore of a fluorescent protein. Examples of the fluorescent protein include proteins having at least 70% homology with the green fluorescent protein (GFP) of Aequorea Victoria and proteins having at least 70% homology with the Discosoma striata marine anemone fluorescent protein (DsRed), or the like.

Examples of the ¹H MRI labeling compounds include lanthanide binding groups, such as DTPA, DOTA, DO3A, metallofullerenes, texaphyrins or the like. The lanthanide binding groups can be complexed with lanthanides like gadolinium to produce a relaxation rate agent or lanthanides like dysprosium to produce a chemical shift agent for use in PARACREST imaging. Other types of ¹H MRI labeling compounds include iron oxide particles, gold nanoparticles, gold nanoshells, and the like. Examples of the PET emitting compounds include any compounds including a PET emitting atom, such as ¹¹C, ¹⁸F, ¹²⁴I, and the like. Examples of the SPECT emitting compounds include any compounds including a SPECT emitting atom, such as ^{99m}Tc, ¹¹¹In, ¹²³I, and the like.

The smart contrast agent can further include a standardized biogroup, such as an antibody, an antigen, a transport protein, a transport peptide, a sugar, a nanoparticle, biotin, thiobiotin, polyethylene glycol, and/or an amlyoid binding compound. The standardized biogroup can be covalently bonded to the first labeling group or to the core peptide, for example.

FIG. 2 illustrates a smart contrast agent, according to another exemplary embodiment of the present invention. The smart contrast agent includes the core peptide, a first labeling group attached to a first end of the core peptide, and a second labeling group attached to a second end of the core peptide. The smart contrast agent can be in the form of an organic salt, a hydrate, or a salt hydrate. The end termini of the smart contrast agent can be in a zwitterionic form or a neutral free amine-acetylated form.

For ease of explanation, the first labeling group is referred to as being attached to the N-terminus of the core peptide, and second labeling group is referred to as being attached to the C-terminus of the core peptide. However, according to the aspects of the present invention, the orientations of the first and second labeling groups, with respect to the N-terminus and C-terminus of the core peptide, can be reversed.

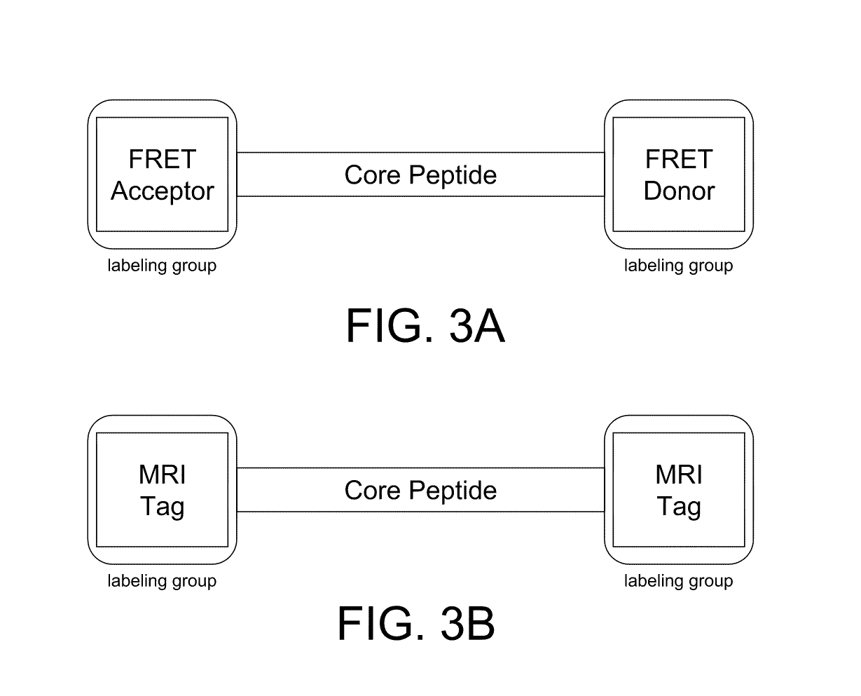

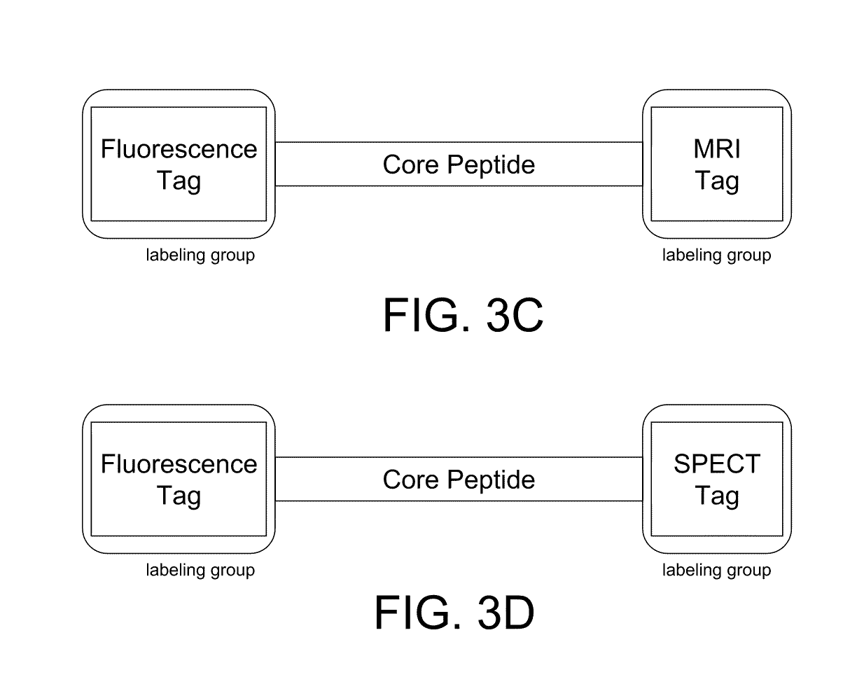

In FIG. 3A, the smart contrast agent includes a FRET acceptor, as a first labeling group, and a FRET donor, as a second labeling group, which are attached to a core peptide. For example, the FRET donor can be 5-carboxyfluorescein (5-FAM), and the FRET acceptor can be 4-(dimethylamino) azobenzene-4′-carboxylic acid (dabcyl), which are shown below with L-lysine linkers denote hereafter as K(5-FAM) and K(dabcyl).

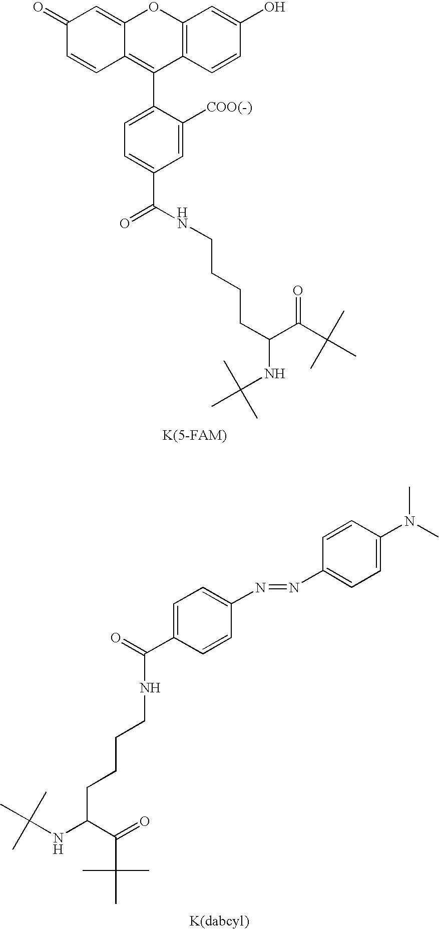

The 5-FAM and the dabcyl groups can be attached to the core peptide, thereby forming an intensity-based fluorescence smart contrast agent, according to an exemplary embodiment of the present invention, which can report on copper and zinc ions. The smart contrast agent would then be capable of undergoing a FRET mechanism, in order to report on changes in a local environment and/or the dynamics or structure of the core peptide. The transition metal once bound to the core peptide can also act as a FRET acceptor group itself, provided the complex has a significant electromagnetic absorption in the visible or near-infrared range. The electrostatic field produced from the positive charge of a transition metal, bound to the peptide, can also cause a red-shifting of both the absorbance and fluorescence spectrums of the labeling groups, providing yet another mechanism to report on the presence of copper or zinc ions.

During a peptide binding event, the average distance and orientation between the donor and acceptor groups is altered, producing a change in the fluorescence spectrum. The intensity of the peptide fluorescence signal is a function of both the target metal ion concentration and the concentration of the peptide itself. Individually, the free 5-FAM group is a commonly used, strong, fluorescence compound, with a molar excitation coefficient from 79,000 to 81,000 M⁻¹ cm⁻¹. Its maximum excitation wavelength is 492 nm and its maximum fluorescence emission wavelength is 515 nm. The fluorescence intensity of the free 5-FAM group is not effected by up to 100 times excess of Cu⁺² ions or a 10 times excess of Zn⁺² ions. The dabcyl group is an efficient absorber (32,000 M⁻¹ cm⁻¹) of visible light, with no emission spectrum. Interaction of the core peptide with the targeted metal ions produces a fluorescence intensity shift that can be used to estimate the concentration of metal ions present, provided an estimate of the smart contrast agent concentration is also available.

Changes in the core peptide structure and the hydration environment of the contrast agent, during target metal ion complex formation, also causes a corresponding detectable change in MRI relaxivity. FIG. 3B illustrates smart contrast agents, according to an exemplary embodiment of the present invention, where the smart contrast agent includes MRI tags as the first and second labeling groups. Other labeling configurations can be selected to produce smart contrast agents with both a fluorescence and MRI/SPECT detection channel. FIG. 3C and FIG. 3D illustrate smart contrast agents, according to an exemplary embodiment of the present invention, where the smart contrast agent includes a fluorescence tag as the first labeling group and either a MRI or SPECT tag as the second labeling group.

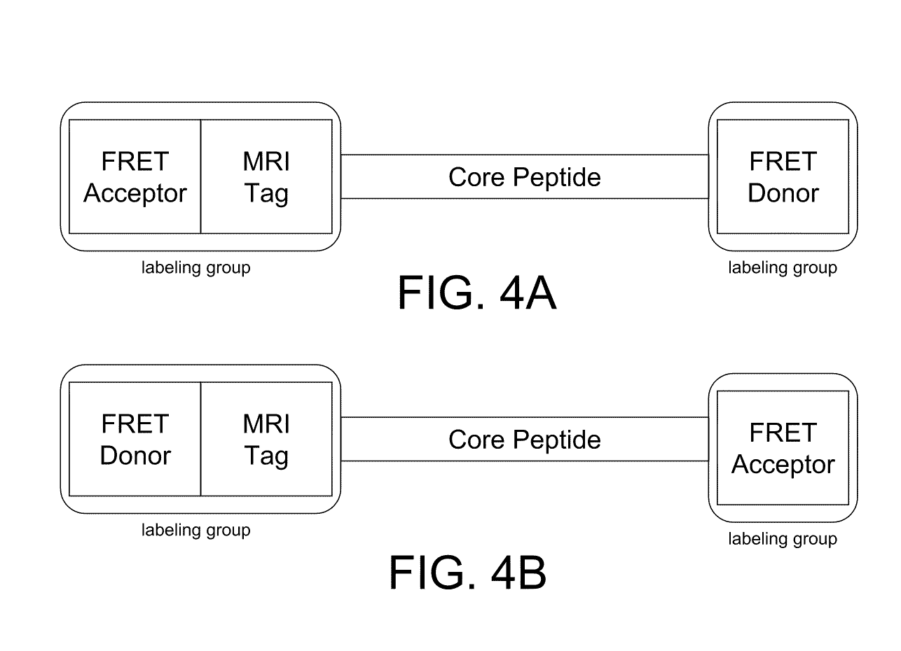

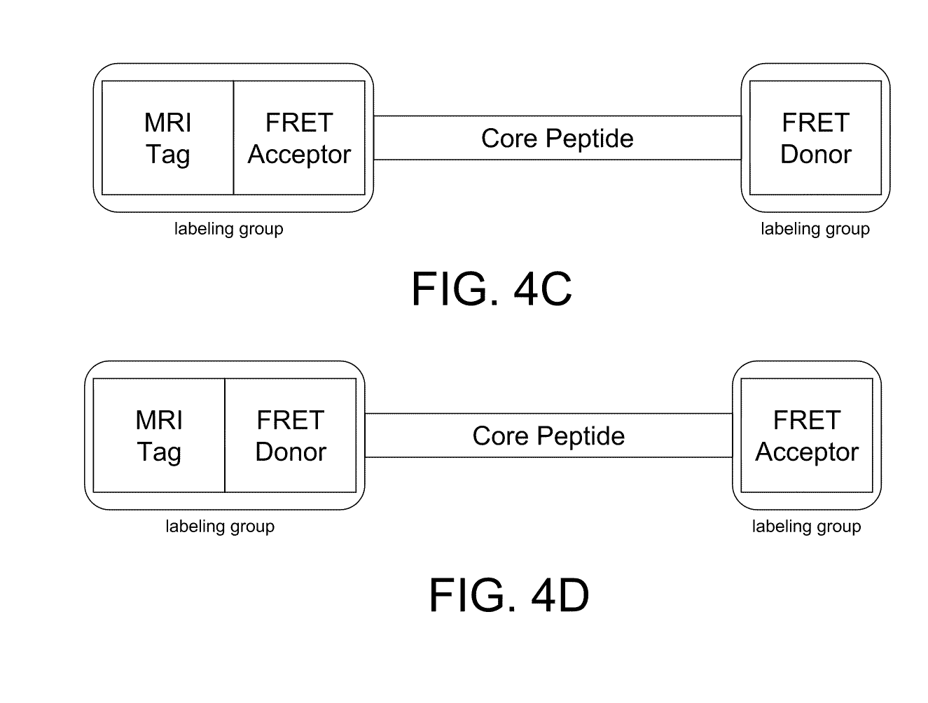

FIGS. 4A-4D illustrate smart contrast agents that include first labeling groups that include two labeling compounds, and second labeling groups that include a single labeling compound, according to exemplary embodiments of the present invention. In FIG. 4A, the smart contrast agent includes a combination of a FRET acceptor and an MRI tag, as a first labeling group, and a FRET donor, as a second labeling group. In FIG. 4B, the smart contrast agent includes a FRET donor and an MRI tag, as the first labeling group, and a FRET acceptor as the second labeling group. In FIGS. 4C and 4D, the sequence order of the FRET donor/acceptor and the MRI tag are reversed, with respect to the sequence order shown in FIGS. 4A and 4B.

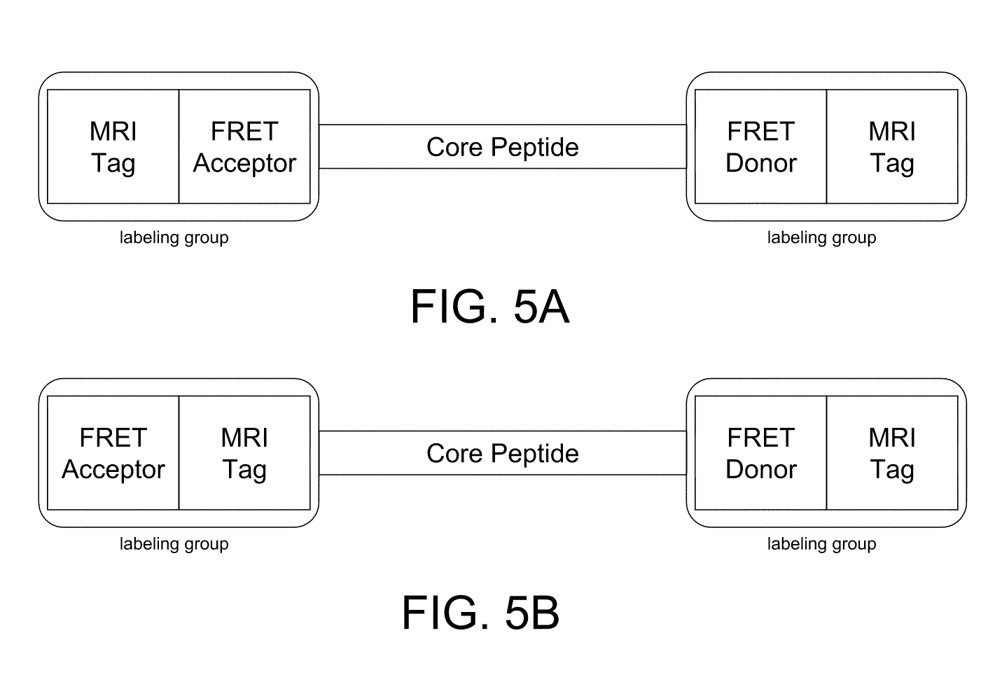

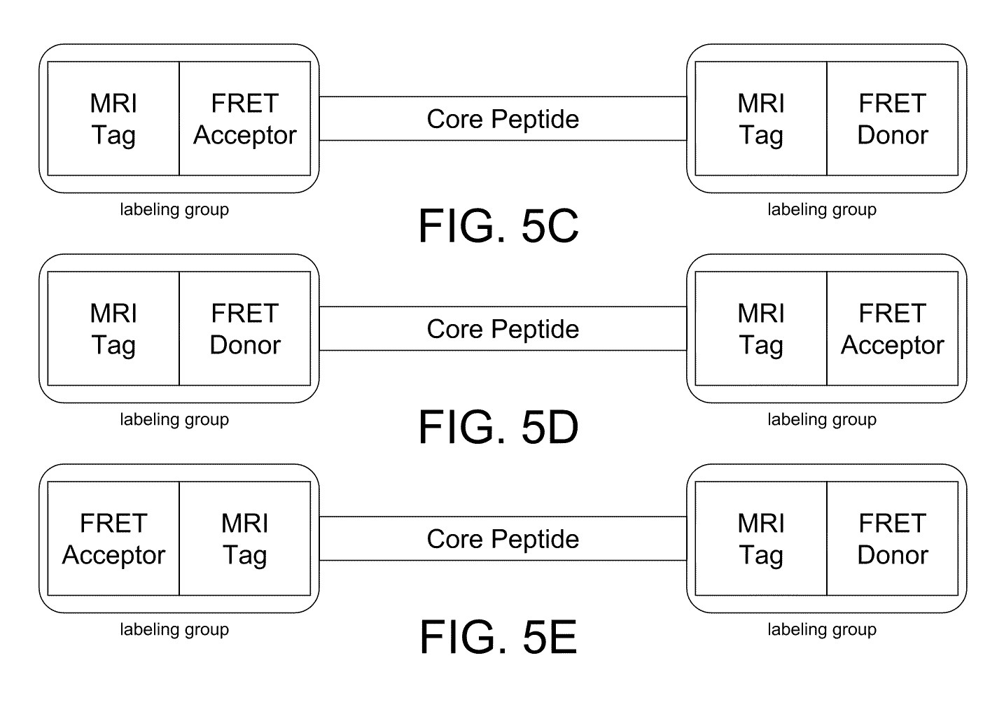

FIGS. 5A-5E illustrate smart contrast agents that include a core peptide and first and second labeling groups that each include two labeling compounds. As shown in FIGS. 5A-5E the first and second labeling groups include various combinations of FRET compounds and MRI tags.

The use of only a single detection channel may not permit both the concentration of the peptide and target metal to be determined simultaneously. The idealized equation, describing the interaction of the smart contrast agent with a single transition metal ion, is given by the reversible chemical reaction [C:M] [symbol] [C]+[M], where C is the contrast agent, M is the metal ion, and C:M is the contrast agent metal ion complex. Defining α=[C:M]/[C]₀ the equilibrium constant of the reaction can be expressed in terms of the fraction of contrast agent molecules complexed to the target transition metal ion by,

$$K = \frac{α}{( 1 - α ) * ( {[ M ]}_{0} - {[ C ]}_{0} α )} \qquad ( Equation 2 )$$

In the above formula, K is the binding affinity, and the subscript 0 referrers to the initial concentrations. The solution for α is then given by:

$$α = \frac{\begin{matrix}1 + {K [ M ]}_{0} + {K [ C ]}_{0} - \\ \sqrt{- 4 {{{K}^{2} [ M ]}_{0} [ C ]}_{0} + {( - 1 - {K [ M ]}_{0} - {K [ C ]}_{0} )}^{2}}\end{matrix}}{2 {K [ C ]}_{0}} \qquad ( Equation 3 )$$

To determine [M]₀ for each voxel or pixel requires knowledge of both α and [C]₀. During imaging, the concentration of peptide across pixels or voxels will generally not be uniform or static, because of biological mechanisms involved in distributing, circulating, and clearing the contrast agent from the tissue or cells being studied. For the purposes of more clearly describing the aspects of the present invention, it is useful to imagine an idealized imaging system, where the primary measurement channel would be engineered to solely be a function of the variable a for each voxel or pixel. The secondary measurement channel would then be engineered to solely be a function of [C]₀ for each voxel or pixel.

According to an exemplary embodiment of the present invention, the first and/or second labeling groups may include an additional detection channel to help determine the local concentration of the smart contrast agent. For example, replacing the dabcyl FRET acceptor in the previous example with another FRET acceptor group having a fluorescence emission spectrum, such as rhodamine red, TAMRA, Texas red, or other suitable FRET acceptor compounds, would impart a second red shifted peak to the fluorescence signal. Measurements of the intensity ratio between the donor and acceptor fluorescence peaks would then report on the degree of complexation described by α in a method that is more independent of the contrast agent concentration and would then represent an improved primary channel. Measurements of the total fluorescence over both the donor and acceptor emission spectra, would then represent an improved secondary channel, providing more direct information about the local concentration of the smart contrast agent. The replacement of the quencher with a fluorescence group would also increase the net amount of photons received by the detector and would improve the detection limit of the smart contrast agent.

A secondary channel can however be omitted, because it is not necessary to determine the exact local concentration of the target transition metal to produce a useful diagnostic tool. The minimum requirement for a diagnostic application is only that the digital image obtained from a diseased tissue contains enough consistently distinct features, as compared to a similar healthy tissue, so as to permit diagnosis by a medical professional.

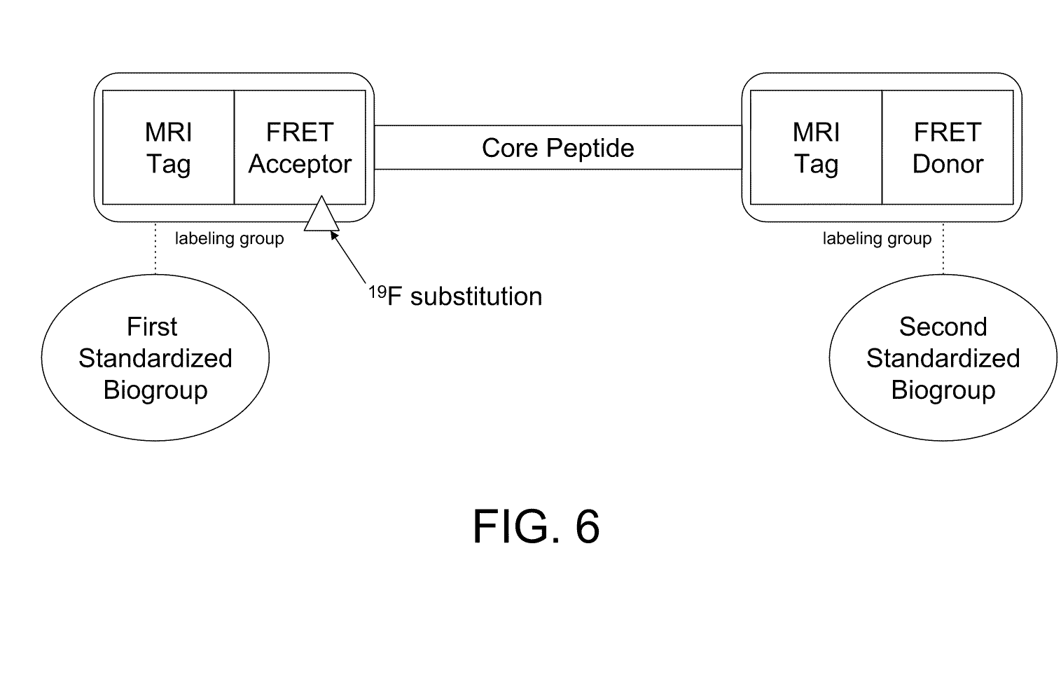

FIG. 6 illustrates a smart contrast agent, according to another exemplary embodiment of the present invention. In FIG. 6, the smart contrast agent includes first and second labeling groups. The first labeling group comprises an MRI tag, a FRET acceptor, and a first standardized biogroup. The second labeling group comprises an MRI tag, a FRET donor, and a second standardized biogroup. In addition, the FRET acceptor compound includes a fluorine substitution, such that the FRET acceptor compound can also be used as a ¹⁹F MRI labeling compound.

The first and second standardized biogroups can be independently selected from an antibody, an antigen, a transport protein, a transport peptide, a sugar, a nanoparticle, biotin, thiobiotin, polyethylene glycol, an amyloid binding compound, or a combination thereof. The first and second biogroups can be covalently bonded to the first and second labeling groups.

In other exemplary embodiments of the present invention, the labeling groups can include various combinations of the ¹H MRI and ¹⁹F MRI labeling compounds, to serve as simultaneous hybrids of both the primary and secondary channels. In this implementation, the signal changes, occurring as a result of transition metal complexation from the ¹H MRI and ¹⁹F MRI labeling compounds, would depend on both the smart contrast agent's local concentration and the amount of contrast agent complexed with the target transition metal. The exact function of the ¹H MRI and ¹⁹F MRI signal dependences would, however, be different. Both α and [C]₀ could be determined for each voxel, by constructing two standardized functions (one for the ¹H signal and one for the ¹⁹F signal) and solving for the two unknown, common variables between them, namely α and [C]₀.

In another exemplary embodiment of the present invention, the core peptide is chemically attached to either a labeling compound with a PET emitting atom, such as ¹¹C, ¹⁸F, or ¹²⁴I; or a labeling compound with a SPECT emitting atom, such as ^{99m}Tc, ¹¹¹In, or ¹²³I, in order to induce an improved secondary detection channel. The intensities of PET and SPECT signals have the advantage of being largely independent of their local chemical environment, under physiological conditions, and can be used to measure the local concentration of the smart contrast agent. The first and second labeling groups could include fluorescence and/or MRI tags, and any combination of either the PET and/or SPECT tags. Dual fluorescence/PET, fluorescence/SPECT, MRI/PET, or MRI/SPECT scans can be used to construct digital maps of the local concentrations of both the core peptide and the targeted metal ion concentration within a sample.

In another exemplary embodiment of the present invention, the core peptide is chemically attached to either a labeling compound with a PET emitting atom, such as ¹¹C, ¹⁸F, or ¹²⁴I; a labeling compound with a SPECT emitting atom, such as ^{99m}Tc, ¹¹¹In, or ¹²³I; a MRI labeling compound containing a lanthanide atom such as gadolinium, europium, dysprosium; or a labeling compound with at least one ¹⁹F atom. Binding of the core peptide with the targeted transition metal ion causes a structural change in the smart contrast agent that alters its solubility and transportation kinetics within a biological sample. PET, SPECT, ¹H MRI, or ¹⁹F MRI imaging of the time dependent accumulation and clearance of the smart contrast agent is then used, as a primary channel, to construct digital maps of the targeted metal ion within the sample.

One aspect of any ¹H MRI contrast agent containing a paramagnetic lanthanide ion, is to assure that the lanthanide ion remains tightly bound under physiological conditions. Free lanthanide ions can be highly toxic and may be dislodged from their intended chelater site, by decreases in pH or binding competition from other transition metals. Thermodynamic stability constants for many compounds that are used as tagging groups in previous smart contrast agents for zinc and copper, as well as several ¹H MRI tags included in aspects of the present invention, have been reported (W P Cacheris, S C Quay and S M Rocklage. The relationship between thermodynamics and the toxicity of gadolinium complexes. Magnetic Resonance Imaging. 1990; 8: 467-81. E Toth, R Kiraly, J Platzek, B Raduchel and E Brucher. Equilibrium and kinetic studies on complexes of 10[2,3-dihydroxy-(1-hydroxymethyl)-propyl]-1,4,7,10-tetraazacyclododecane-1,4,7-triacetate. Inorganica Chimica Acta. 1996; 249: 191-99. K Kumar, A Chang, L C Francesconi, D D Dischino, M F Malley, J Z Gougoutas and M F Tweedle. Synthesis, stability, and structure of gadolinium(III) and yttrium(III) macrocyclic poly(amin carboxylates). Inorganic Chemistry. 1994; 33: 3567-75. G Anderegg, F Amaud-Neu, R Delgado, J Felcman and K Popov. Critical evaluation of stability constants of metal complexes of complexones for biomedical and environmental applications. Pure and Applied Chemistry. 2005; 77: 1445-95). Knowledge of the stability constants enables equilibrium predictions regarding the completeness of the complexation with various lanthanides, like gadolinium. The effectiveness of competitive binding reactions from other transition metals, likely to be encountered during in vivo applications, can also be estimated. Solving of the equilibrium equations for dilute 1:1 mixtures of the chelater compounds and gadolinium ions reveal that the fraction of gadolinium ions bound at physiological pH is near 100%. Significant release of gadolinium ions from the chelater sites does occur under acidic conditions, when the pH approaches 4-4.5 units, as shown in the following Table 3.

TABLE 3
Estimated pH required to yield a 0.01%, 0.1% or 1.0% release of gadolinium ions

| | EDTA | DTPA | DTPA-BMA | DO3A-B | DO3A | DOTA |
|---|---|---|---|---|---|---|
| 0.01% release | 5.4 | 4.3 | 4.5 | 5.9 | 5.2 | 4.5 |
| 0.10% release | 4.4 | 3.6 | 3.4 | 4.9 | 4.3 | 3.9 |
| 1.00% release | 3.4 | 2.9 | 2.7 | 4.1 | 3.6 | 3.3 |

Introduction of equal molar amounts of copper and/or zinc to these solutions, however, can dramatically affect the system equilibrium even at physiological pH. Copper and zinc are known to strongly compete with gadolinium ions for the chelater sites in compounds like ethylenediaminetetraacetic acid (EDTA), DTPA and DO3A-B, as shown in the following Table 4.

TABLE 4
Hypothetical percentage of free gadolinium ions at pH = 7.4

| | EDTA | DTPA | DTPA-BMA | DO3A-B | DOTA |
|---|---|---|---|---|---|
| | (200 μM) | (200 μM) | (200 μM) | (200 μM) | (200 μM) |
| 200 μM Gd⁺³ | 0.0004% | 0.000006% | 0.0003% | 0.0003% | 0.000008% |
| 200 μM Gd⁺³ + | 86.2% | 22.4% | 1.2% | 61.3% | 5.9% |
| 200 μM Cu⁺² | | | | | |
| 200 μM Gd⁺³ + | 38.4% | 1.3% | 0.4% | 12.4% | 1.1% |
| 200 μM Zn⁺² | | | | | |

Table 4 shows the predicted amount of gadolinium exchange expected for a few hypothetical solutions of 0.2 mM chelater+0.2 mM Gd⁺³+0.2 mM of either Zn⁺² or Cu⁺². The actual stabilities of any ¹H MRI tagging group would also likely be significantly affected by the chemistry of other neighboring functional groups within the smart contrast agent, and thus, the thermodynamic stabilities presented in Table 4 may not be high quality predictors of the degree of lanthanide release observed for a specific agent. These predictions also exclude the affects of any reactions of the metal ions with metalloproteins present during in vivo applications. Nevertheless, these predictions are still useful illustrations of the possible limitations imposed from undesirable metal exchange reactions on the practical application of smart ¹H MRI constants agents for transition metals. Compounds like EDTA, DTPA, and DO3A-B have a strong degree of ion exchange, while compounds like DTPA-BMA and DOTA are considerably more stable toward the bound gadolinium complex. The idealized smart contrast agent for transition metals, employing a ¹H MRI tag, incorporates a MRI tagging group that is several orders of magnitude more stable toward the paramagnetic lanthanide complex than analogous complexes with the targeted transition metals. Metallofullerenes are also possible candidates to reduce undesirable metal exchange processes, because metallofullerenes typically require high temperatures, far in excess of those found in physiological conditions, to remove significant amounts of the trapped lanthanide atoms from their fullerene cages.

Besides significant increases in toxicity, gadolinium release can also cause misinterpretations of relaxivity data, if not properly considered. Under a magnetic field strength of 1.0 T, the r₁ and r₂ relaxation times of the aqua Gd⁺³ ion is 9.1 mM⁻¹ s⁻¹ and 10.3 mM⁻¹ s⁻¹, respectively. These relaxation rates are about 2.5 times higher than those from DTPA/Gd⁺³, DOTA/Gd⁺³, DTPA-BMA/Gd⁺³ and HP-DO3A/Gd⁺³ complexes, so any release of Gd⁺³ atoms from the chelater site can significantly contribute to the net sample relaxivity. Nuclear relaxation measurements using mixtures of Gd⁺³ and transition metal ions (in particular Cu⁺² and Zn⁺² ions) should be made in consideration of the effect of metal exchange processes, and the affect that free aqua Gd⁺³ ions could have on the measured relaxation times.

According to another aspect of the present invention, the smart contrast agent can be used as a therapeutic treatment of disease, by the metal chelation of either zinc or copper. For example, amyloid plaques, which play a central role in the pathology of Alzheimer's disease, are known to sequester copper, zinc, and iron (M A Lovell, et al. 1998). The reduction of the bioavailability of copper and zinc is also known to reduce the activity of amyloid degrading enzymes (D Strozyk, et al. 2007), potentially leading to further decreases in zinc/copper bioavailability and creating a feedback mechanism favoring run-away amyloid accumulation. The copper binding affinities of K(FAM)PHGGGWGQK(dabcyl) (P1P) and Ac-K(6-FAM)PHGGGWGQP(CF₃)—NH₂ (P50), for instance, are on the low μM to nM scale and are strong enough to compete for copper atoms bound to amyloid plaques. The chelation and removal of zinc and copper atoms from the amyloid plaques can thereby help break up and dissolve the deposit, freeing the metal ions to further enhance the activity of amyloid degrading enzymes.

Increasing the bioavailability of intracellular copper has been shown to restore cognitive function in rodents by inhibiting the accumulation of β-amyloid trimers and phosphorylated tau (P J Crouch, et al. 2009). Increasing bioavailability of copper or zinc inside the brain is however difficult because passage of these transition metal ions into the brain is highly regulated by a complex series of proteins. In another exemplary embodiment of the present invention, the contrast agent can also be made to act as transition metal delivery system capable of transcending the blood-brain barrier. This is accomplished by binding the agents with their target metal prior to delivery into the body. The agent/metal complex can then cross the blood brain barrier, releasing the target metal inside the brain and helping to stimulate the activity of amyloid degrading enzymes.

Another example of the current invention relates to the treatment of copper overload diseases such as Wilson's disease (R Kitzberger, et al. 2005). Extrapyramidal symptoms are the predominant neurological feature of Wilson's disease and are caused by excess accumulation of copper in the liver, eventually leading to an overload of copper inside the brain. There are three main drugs currently approved for the treatment of copper overload disorders: zinc salt, penicillamine and trientine (M L Schilsky 2001). Zinc salts, such as zinc chloride, zinc sulfate, or zinc acetate block the absorption of copper by triggering the production of metallothionein proteins. The clearance rate of copper using zinc salts however is low and therefore is used primarily as a maintenance therapy, rather than initial treatments in symptomatic patients. Penicillamine is a copper chelator and promotes the excretion of copper in the urine. However, the side effects of penicillamine are significant and usually lead to the discontinuation of use by 10-20% of patients. Trientine is another copper chelator and has a better safety profile than penicillamine, but is still known to result in anemia, systemic lupus, bronchitis and asthma. In another example of the current invention, the contrast agent can function as a strong and selective chelator of copper ions, removing excess copper from both the circulatory and central nervous systems. The advantage of the present teachings over existing drugs is that, because agent is derived from the commonly expressed prion protein, prolonged use should be better tolerated.

In another example of the current invention, the contrast agent can be used as a therapeutic drug against several types of neurological disorders that exhibit pathologies with altered transition metal homeostasis. These include Alzheimer's disease, Parkinson's disease, dementia with Lewy bodies, bipolar disorders and depression. In addition to those examples already mentioned, there are numerous other disorders where a transition metal chelator or transition metal delivery system could have profound usefulness as a therapeutic treatment. For example, an associative link between the deregulation of serum copper has also been found with declines in cognitive function during Alzheimer's disease (R Squitti, et al. 2009). Concordant bicompartmental dopaminergic deficits in neurologic Wilson disease have also been observed (H Barthel, et al. 2003), suggesting that the pathologies of Wilson's disease and Parkinson's disease are related. The aggregation of α-synuclein into Lewy bodies, one of the hallmark features of both Parkinson's disease and dementia with Lewy bodies, is also copper dependent, arising from strong affinities of α-synuclein to copper. The potential formation of a neurotoxic copper-dopamine complex that is recognized by dopamine transporters could also provide a possible pathway for the selective death of dopamine active cells (I Paris, et al. 2001). Bipolar disorders have an exceptionally high level of serum copper and other transition metal abnormalities (M S Mustak, et al. 2008). Zinc homeostasis is also known to be altered during depression (G Nowak, et al. 2005).

### Example 1

### A Fluorescent Agent that Includes 2 Labeling Groups

The compound used in this example will be referred to as P1P and includes the following sequence, K(FAM)PHGGGWGQK(dabcyl).

P1P was chemically synthesized, in its zwitterionic form, using standard Fmoc/tBu methods (W C Chan, et al. 2000). The K(FAM) and K(dabcyl) groups were attached to the side chain of each lysine residue, prior to their attachment to the core peptide (PHGGGWGQ). Lysine peptide building blocks incorporating FAM and dabcyl on to the side chain are commercially available as Fmoc-Lys(5-FAM)-OH and Fmoc-L-Lys(dabcyl)-OH. These building blocks were used in the synthesis procedure, using standard Fmoc/tBu techniques for peptide synthesis. The quality of the commercially available building block, Fmoc-Lys(5-FAM)-OH, can vary and a direct separation of potential isomeric K(FAM) variations in the peptide product by HPLC was not possible, because of similar column retention times. The fluorescence properties of the K(FAM) labeling group itself, however, are largely unaffected by the possible isomeric variations. Synthesis schemes that attempted to add the FAM or dabcyl groups during a post core peptide attachment step failed to produce acceptable product yields, because of poor selectivity of the reaction to the targeted lysine side chain.

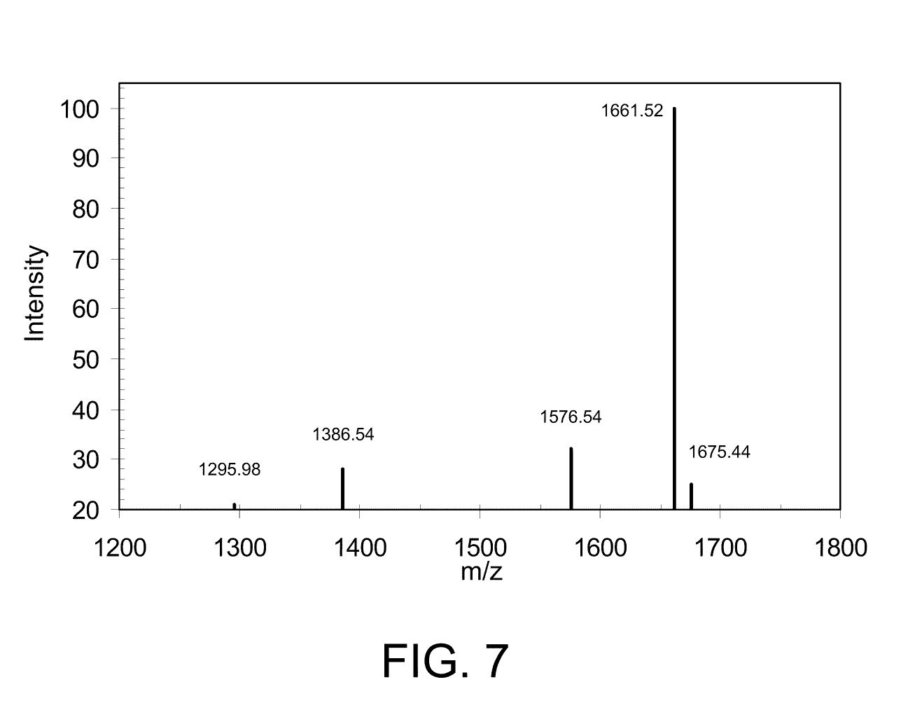

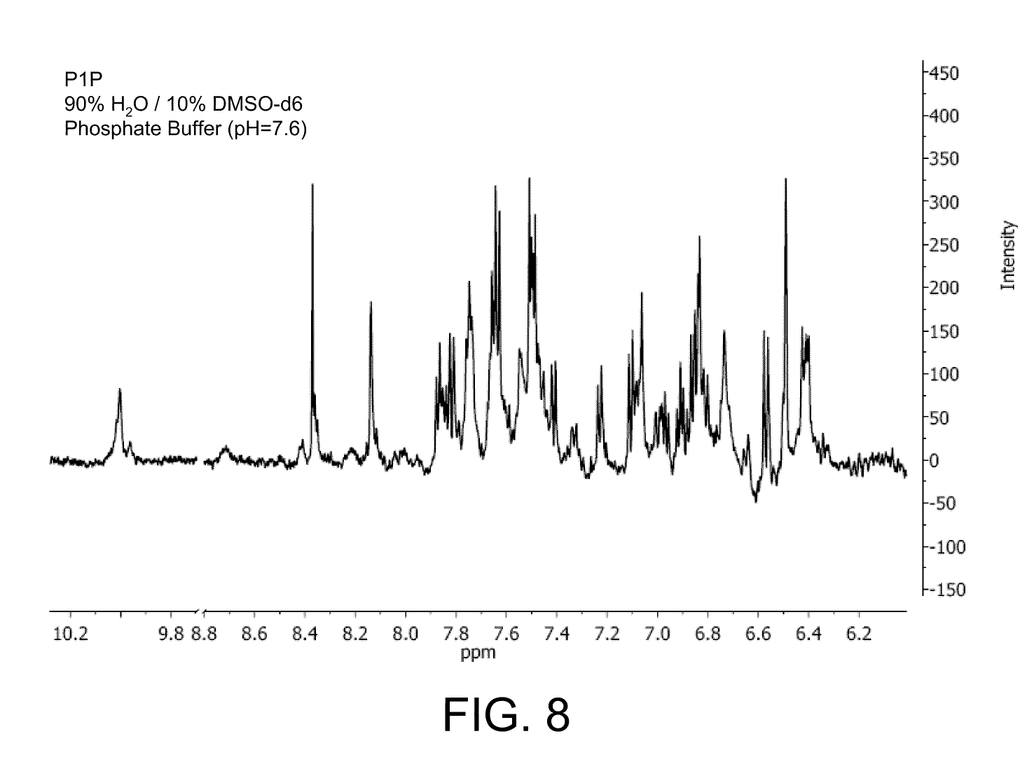

The crude peptide product was purified using reverse-phase HPLC and C18 column. Purification was continued until >95%, as confirmed by HPLC and electrospray mass spectrometry, as shown in FIG. 7. The primary peptide impurity, as confirmed by tandem mass spectrometry, was determined to contain an additional methyl group located within the K(FAM) residue and this impurity is evidenced by the 1675 m/z peak in FIG. 7. To better characterize the chemistry of P1P, a high resolution 600 MHz ¹H NMR scan of the peptide, in a 90/10% H₂O/DMSO-d6 solvent mixture, was conducted using a sodium phosphate buffer (pH=7.6). The ¹H NMR spectrum was obtained at room temperature, by averaging 8196 individual NMR scans. The hydrophilic region of the spectrum is shown in FIG. 8.

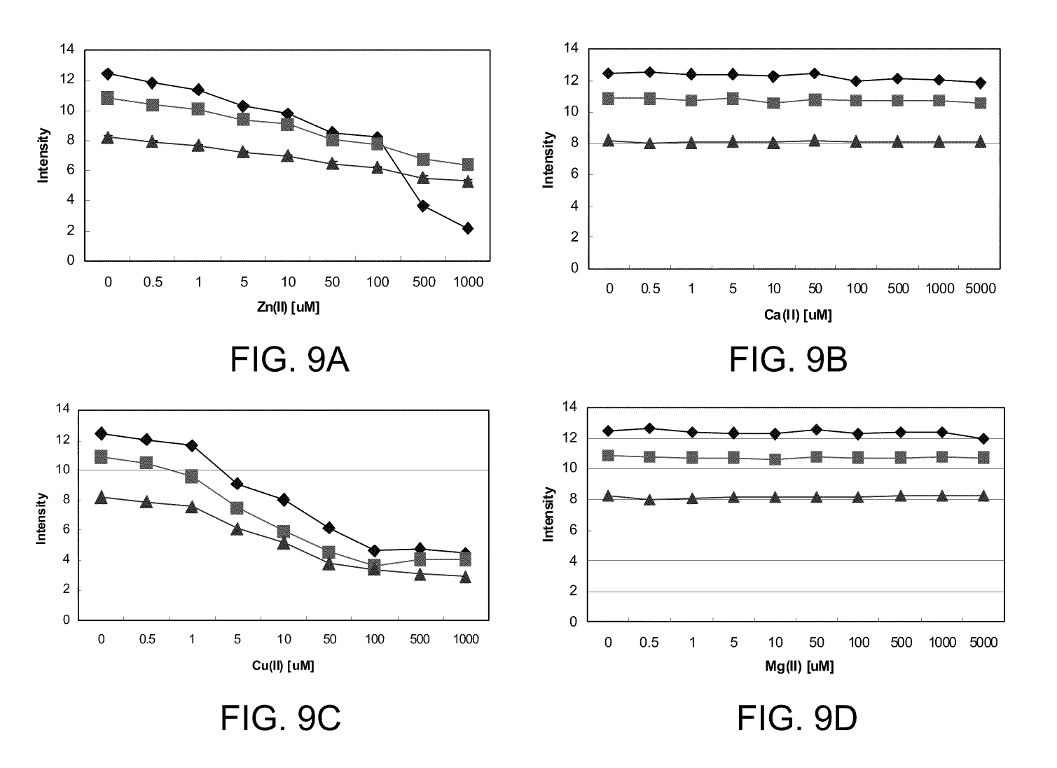

Fluorescence titrations were used to determine the ability of P1P to bind with various cations in aqueous solutions of 10 μM P1P, 20 mM buffer and 2% DMSO. These solutions were titrated with CaCl₂, MgCl₂, MnCl₂, FeCl₂, CuSO₄, and ZnCl₂ (FIGS. 9A-9D). The fluorescence was measured with a Fluoroskan FL machine. The excitation filter wavelength was 485 nm and the fluorescence emission filter was 520 nm. The solutions were buffered at pH=6.4, pH=7.0, and pH=7.6. The solutions were tested under various buffer conditions using 20 mM of either m-ethylmorphine (NEM), tris (hydroxymethyl) aminomethane (TRIS), 4-(2-hydroxyethyl) piperazine-1-ethanesulfonic acid (HEPES), 4-morpholinoethanesulfonic acid (MES), and 4-morpholinepropanesulfonic acid (MOPS) as appropriate. The pH was adjusted to the desired level by using small additions of either HCl or NaOH. The fluorescence of each condition was tracked over a period of 120 minutes, to insure equilibrium was obtained. After 120 minutes the pH of each condition was double checked again with pH paper, to confirm the pH remained constant during the experiment.

The fluorescence intensity of FAM strongly depended on the pH and decreased rapidly at pH values of less than 7. The decrease in the baseline fluorescence intensity of the P1P, which decreased as pH decreased, was largely due to the strong pH dependence of the FAM fluorescence intensity. Test solutions that showed a change from the desired pH (typically solution containing high amounts of metal salts) were discarded from analysis. NEM solutions containing more than 0.1 mM CuSO₄ or ZnCl₂ also showed the presence of faint suspensions or salt residues collecting on the walls of the well plate. This material formed slowly, over tens of minutes, and did not seem to affect the fluorescence results. The material formed in the absence of peptide, and rerunning the experiments with an alternative buffer of 20 mM TRIS prevented the formation any visible particles or residue. The fluorescence P1P titration results were largely unchanged by the use of a TRIS buffer, with the exception of a minor effect at the higher copper concentrations. TRIS is well known to bind copper ions and this effect is presumably caused from competition of the peptide with the TRIS buffer.

The results show that P1P has no fluorescence change in the present of large excesses of Ca⁺², Mg⁺² and Fe⁺² indicting P1P does not interact with these ions. These properties relate to practical applications, because inside the body the concentration these ions are likely to exceed the concentration of copper and zinc, and any significant affinity for Ca⁺², Mg⁺², or Fe⁺² could create significant background interference in the imaging signal. A weak interaction of P1P with Mn⁺² was observed at 100 times the P1P concentration, however this is not expected to cause a significant amount of signal interference, because the concentration of Mn⁺² is generally much less that of zinc and copper ions inside the body.

FIGS. 9A-9D show fluorescent titration results of Cu⁺², Zn⁺², Mg⁺², and Ca⁺² binding to P1P. The reaction with P1P with Cu⁺² was strong at all pH values studied. To estimate the binding affinities a simple theoretical model was constructed. In the model only two states of the P1P peptide were assumed to exist, namely the metal bound and metal unbound state. Each state has a characteristic fluorescence, denoted by f_{bound} and f_{unbound}, and whose intensity increases linearly with increasing molecular concentration in accordance with Beer's Law of dilute solutions. A combination of bound and unbound peptide populations yields a net fluorescence intensity, I, of

I=f_{bound}α+i f_{unbound}(1−α) (equation 4).

Substituting Equation 3 for a permits the fitting of a three parameter model (f_{bound}, f_{unbound}, K) describing the fluorescence behavior of the peptide interacting with a transition metal. This model found to describe the interaction of P1P with Cu⁺² well and yielded binding constants of 5.1 μM±3.6 μM, 2.0 μM±3.0 μM, and 17.8 μM±5.5 μM at pH 6.4, 7.0, and 7.6 respectively. These binding affinities are significantly stronger than other ¹H MRI smart contrast agents reported in the literature for copper. The reaction of P1P with Zn⁺² was considerably weaker than Cu⁺², but still significant.

The mechanism of copper binding P1P was examined by synthesizing three mutant versions of P1P, namely P16, P17, and P18. The synthesis method was the same as P1P and the sequences of these mutant peptides are provided in Table 5.

TABLE 5
List of Peptide Sequences Used

| Name | Sequence |
|---|---|
| P1P | K(FAM)PHGGGWGQK(dabcyl) |
| P15 | K(DO3A)K(FAM)PHGGGWGQK(dabcyl)K(DO3A) |
| P16 | K(FAM)PHGGGAGQK(dabcyl) |
| P17 | K(FAM)PAGGGWGQK(dabcyl) |
| P18 | K(FAM)PHGGGWGAK(dabcyl) |
| P20 | Ac-K(5-FAM)PHGGGWGQPHGGGWGQK(dabcyl)-NH₂ |
| P31 | (5-FAM)-KPHGGGWGQK(dabcyl) |
| P41 | K(6-FAM)PHGGGWGQK(dabcyl) |
| P42 | [dK(5-FAM)]PHGGGWGQK(dabycl) |
| P43 | K(5-FAM)[dP]HGGGWGQK(dabycl) |
| P44 | K(5-FAM)P[dH]GGGWGQK(dabycl) |
| P45 | K(5-FAM)PHGGG[dW]GQK(dabycl) |
| P46 | K(5-FAM)PHGGGWG[dQ]K(dabycl) |
| P48 | [dK(6-FAM)]PHGGGWGQK(dabycl) |
| P54 | Ac-K(6-FAM)PHGGGWGQP-NH₂ |
| P58 | Ac-PHGGGWGQPK(6-FAM)-NH₂ |
| P50 | Ac-K(6-FAM)PHGGGWGQP(CF₃)-NH₂ |
| P57 | Ac-K(6-FAM)PHGGGWGQK(DO3A)-NH₂ |
| P1S | K(5-FAM)PHGGGWGQK(dabcyl) |

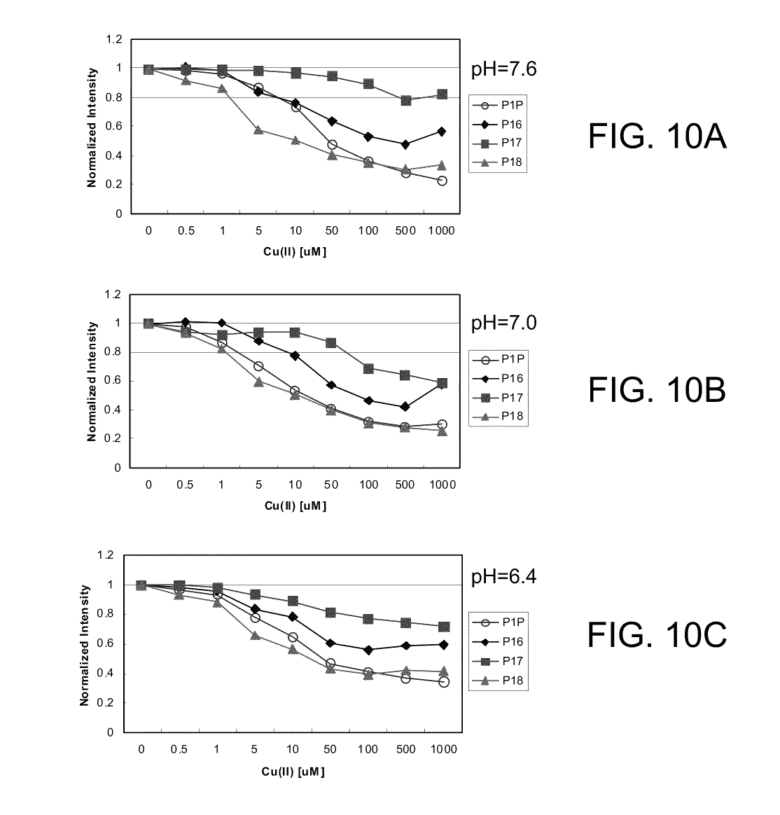

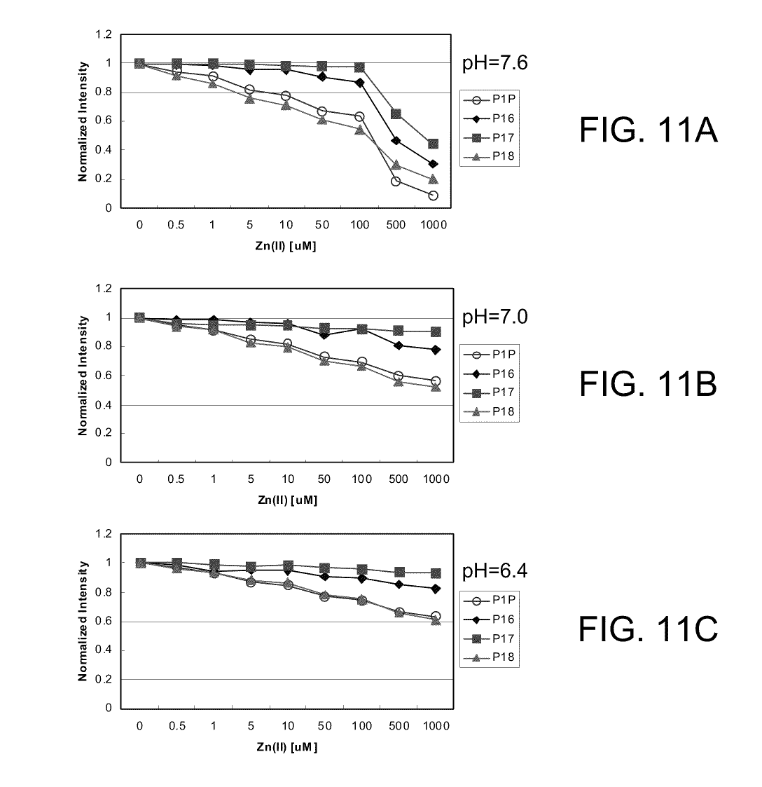

FIGS. 10A-10C show fluorescent titration results of Cu⁺² binding to P16, P17, and P18. FIGS. 11A-11C show fluorescent titration results of Zn⁺² binding to P16, P17, and P18. The results showed that the histidine residue was the most critical to copper and zinc binding activity. The tryptophan residue also had a significant effect on the binding affinities for copper and zinc, but to a lesser extent as compared to the histidine residue mutation. Mutation of the glutamine residue to alanine had little effect on the binding affinity for copper and zinc at pH≦7. Interestingly, the replacement of glutamine with alanine seemed to increase the binding activity of P18 with copper and zinc, at pH=7.6. This mutation could represent a more optimized smart peptide contrast agent. No binding with Ca⁺² or Mg⁺² was found with either the P16, P17, or P18 peptides.

The stability of P1P inside the body is also an important factor. It is possible that proteins and enzymes inside the body could adversely react with P1P, causing premature degradation of the P1P peptide. To test the practically of using P1P in vivo, solutions of P1P in fetal bovine serum and a control protein free phosphate buffer solution were prepared. The solutions were then incubated for a period of 2 hours at 37° C.

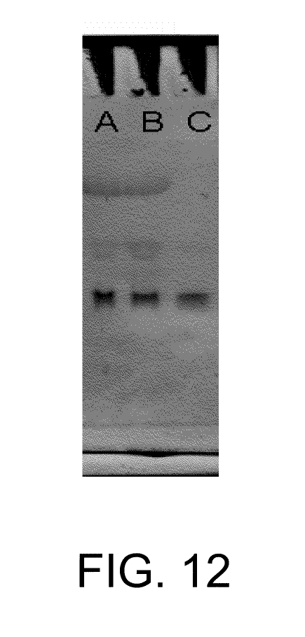

As shown in FIG. 12, an SDS-PAGE procedure was preformed to check if any fragmentation bands were present, thereby indicating that a degradation reaction was occurring in animal serum. For the condition of 0 hour exposure, the P1P peptide sample was mixed with serum at room temperature, but then quickly treated with an enzyme stop solution within 2 minutes of exposure. No fragmentation was detected in either the 0 hour or 2 hour exposure conditions. Similar results were found for P16, P17, and P18.

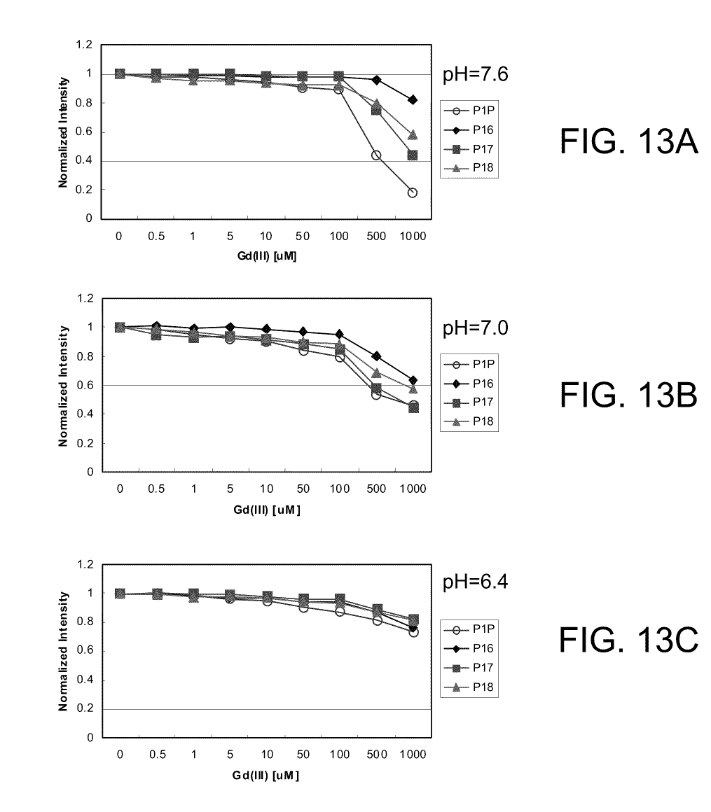

Application of the current invention as a ¹H MRI contrast agent can be accomplished with the chemical addition of lanthanide binding groups, such as DO3A, DOTA, or DTPA, to impart significant increases in the P1P base relaxivity. Cross-reactions between the Gd⁺³ binding moieties and the core peptide copper and zinc binding sites are undesirable. To achieve the lowest levels of toxicity and highest levels of targeted metal selectivity it is important that any cross-reactions between the different metal binding sites remain as weak as possible. FIGS. 13A-13C show the effect on FRET activity for the titration of P1P, P16, P17, and P18 with Gd⁺³ ions. There was a marked increase in Gd⁺³ binding with increasing pH.

The order of important of P1P residues to binding with Gd⁺³ ions was found to be: tryptophan>glutamine>histidine. Interactions of Gd⁺³ ions with the aromatic rings of 5-FAM and dabcyl are also likely non-negligible. We have shown that the glutamine to alanine mutation comprising the P18 peptide either had no effect, or even increased the peptide binding strength with copper and zinc ions. This mutation was also found to reduce the affinity of Gd⁺³ ions to the base P1P peptide. Therefore, one aspect of the present invention encompasses the replacement or deletion of the glutamine residue from the octarepeat unit, in order to decrease unwanted Gd⁺³ cross-reactions and improve copper/zinc binding affinities.

### Example 2

### An Agent that Uses Both a Fluorescent and ¹H MRI Detection Channels

The compound used in this implementation of the invention will be referred to as P15 and has the following sequence, K(DO3A)K(FAM)PHGGGWGQK(dabcyl)K(DO3A), where the K(DO3A) group denotes a DO3A group attached to the peptide backbone through a L-lysine linker, as shown below.

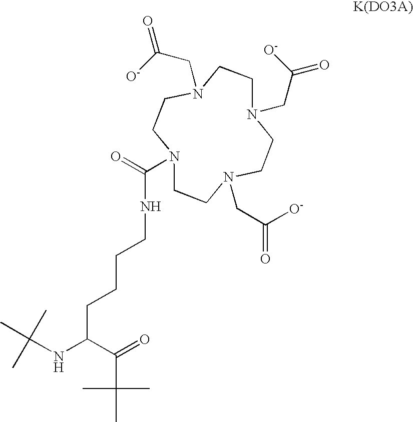

P15 was chemically synthesized, in its zwitterionic form. The attachment of the K(FAM), K(dabcyl), and K(DO3A) groups however necessitated the FAM, dabcyl and DO3A groups to be attached to the side chain of each lysine residue, prior to their incorporation into the backbone chain. Lysine peptide building blocks incorporating FAM, dabcyl, and DO3A onto the side chain are commercially available as Fmoc-Lys(5-FAM)-OH, Fmoc-L-Lys(dabcyl)-OH, and Fmoc-L-Lys(DO3A)-tris(t-Bu)—OH. These building blocks were used in our synthesis procedure using standard Fmoc/tBu techniques for peptide synthesis. The crude peptide product was purified using reverse-phase HPLC and a C12 column. Purification of the peptide was continued until the purity of the product reached >90%, as confirmed by HPLC and MALDI mass spectrometry. The peptide content in the gross product was estimated at 73% from spectrograph analysis, at the FAM absorbance wavelength (492 nm).

To prepare the ¹H MRI active form of P15 Gd⁺³ ions were complexed into the DO3A center of the K(DO3A) residues. A 1.0 mM solution of P15 was prepared using ultra pure water, and the pH was then adjusted to 8.0 using small additions of NaOH. Two molar equivalents of Gd⁺³ ions were added to the P15 solution, from a standardized solution of 1.0 M GdCl₃. The solution was allowed to incubate in the dark for 2 hours, at 30° C. The final pH of the solution was determined to be ˜6.0, predicting that >99% of the Gd⁺³ ions were complexed with the K(DO3A) residues. The peptide solution was then freeze-dried for later use and storage, at −80° C.

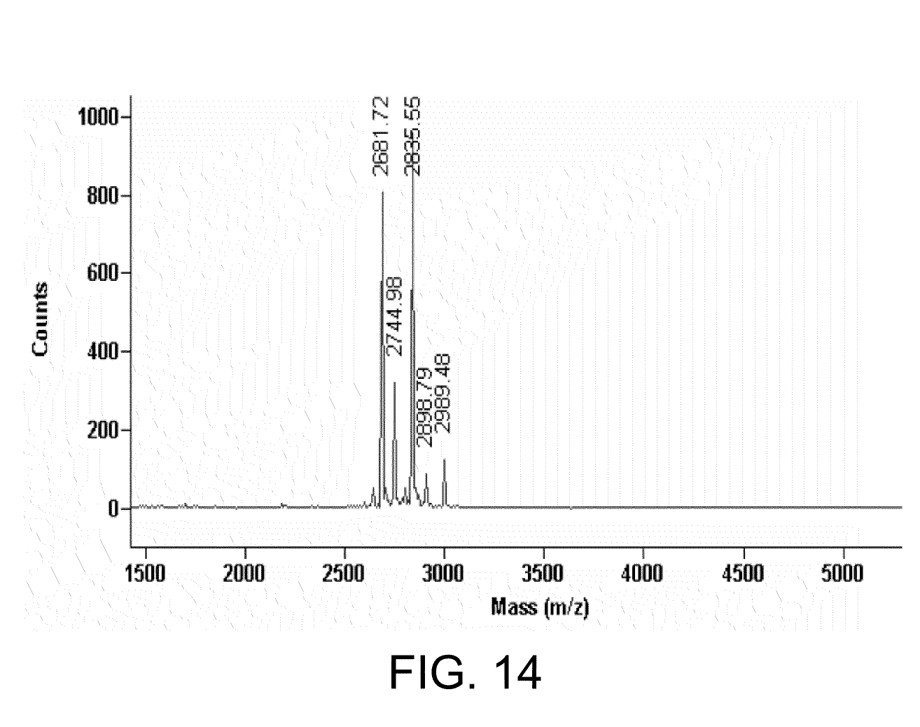

To confirm the successful coordination of the Gd⁺³ ions with the DO3A centers, a small amount of the P15/Gd dried mixture was re-dissolved using only water. This sample was analyzed using MALDI mass spectrometry and HPLC. The MALDI mass spectrum was conducted using the neutral form of sinapinic acid as the matrix, on a standard steel plate. Sinapinic acid is a diprotic acid with pKa values of 4.4 and 9.2. The resultant matrix solutions had a pH near 6.8. The mass spectrum of the negative ion mode is reported in FIG. 14. The theoretical masses and charges of P15 and various Gd⁺³ and Zn⁺² complexes are provided in the following Table 6.

TABLE 6
MALDI Mass Spectrum Peaks of P15

| | Theoretical | Theoretical | Theoretical | Observed | |
|---|---|---|---|---|---|
| | Solution | Solution | [−1] Ion | MALDI | Instrument |
| Species | Charge | Mass | Mass | Mass | Bias |
| P15 | −7 | 2681.74 | 2687.78 | 2681.72 | −6.06 |
| P15 + Zn(II) | −5 | 2747.15 | 2751.18 | 2744.98 | −6.20 |
| P15 + Gd(III) | −4 | 2838.99 | 2842.01 | 2835.55 | −6.46 |
| P15 + Zn(II) + Gd(III) | −2 | 2904.40 | 2905.41 | 2898.79 | −6.62 |
| P15 + 2 Gd(III) | −1 | 2996.24 | 2996.24 | 2989.48 | −6.76 |

The results indicate the P15 peptides can successfully complex with up to 2 Gd⁺³ ions. Additional complexation with Zn⁺² impurities was also observed. The same sample was also rerun using 0.1% TFA in the matrix formulation (to lower the pH significantly). No significant deviation in the spectrum peak positions or relative intensities was seen. It is likely that the Zn⁺² ions are also coordinated with the DO3A centers, because DO3A is expected to also exchange Gd⁺³ with Zn⁺² ions. The degree of metal exchange for the P15 smart contrast agent, however, is not currently well understood. The source of the zinc ion impurities is also unknown, but may be from exposure to the steel MALDI absorption plate, or ion impurities in the reconstituting water or matrix. The instrument bias of 6-7 atomic mass units represents a 0.2% error in the total mass and is within the expected sensitivities of the MALDI mass spectrometer.

The presence of zinc bound impurities in the MALDI spectrum highlights a potential limitation that metal exchange with the K(DO3A) residue may have. This limitation, however, is reduced in alternative implementations of the present invention, by replacing the DO3A group with another ¹H MRI label compound possessing a higher affinity for Gd⁺³, as compared to Zn⁺² and/or other physiologically abundant transition metals. Some examples include, but are not limited to DOTA, DTPA-MPA, and metallofullerene labeling compounds. Provided their size does not adversely affect the biodistribution or core peptide activity, additional alternative implementations of the current invention could utilize iron oxide particles, gold nanoparticles, and/or gold nanoshells, as ¹H MRI label compounds.

It is also possible that Gd⁺³ complexation at the K(DO3A) sites are hindered by the close proximity of the K(FAM) and/or K(dabycl) residues. Additional spacer/linker groups or amino acids (i.e., like glycine or alanine), between the K(DO3A) and K(5-FAM) residues, or between the K(DO3A) and K(dabycl) residues can be used to improve the stability of the labeling groups toward gadolinium.

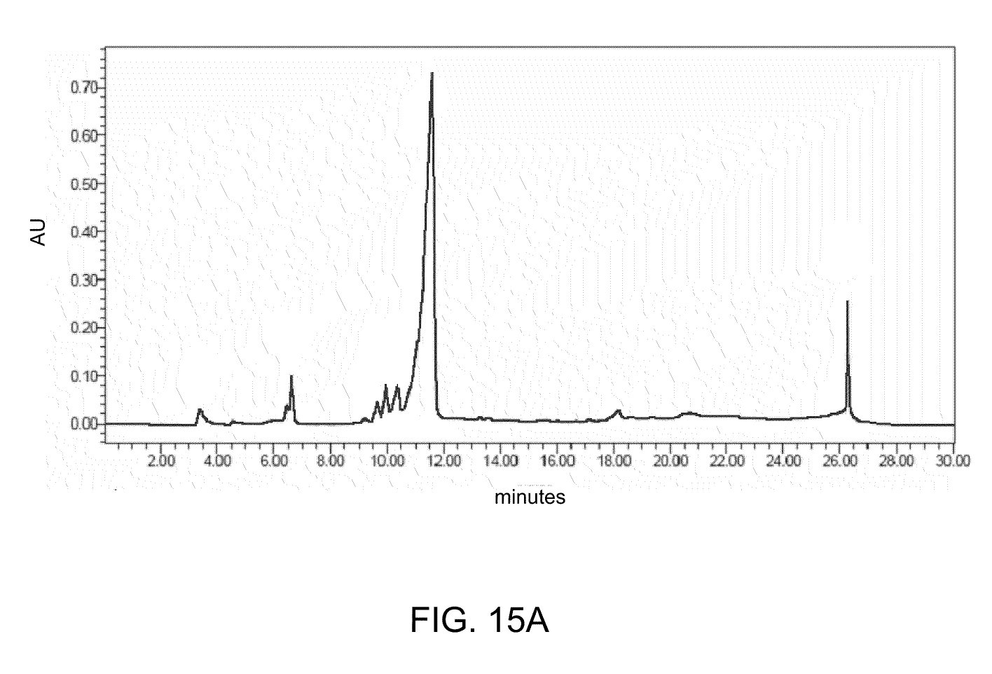

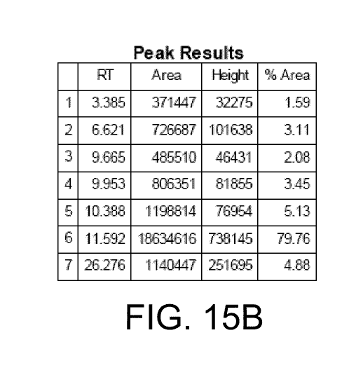

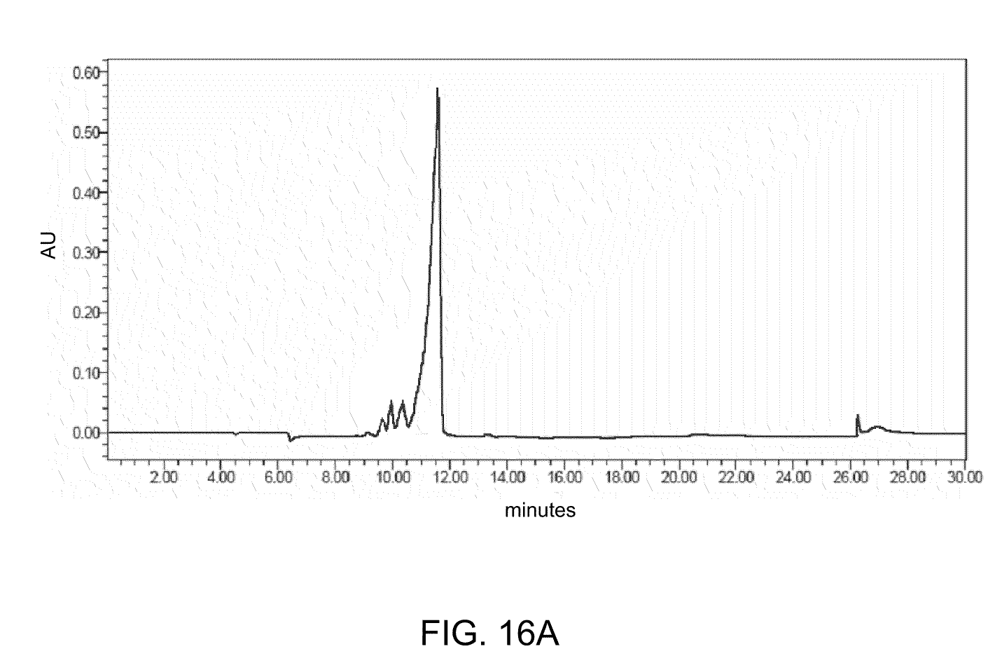

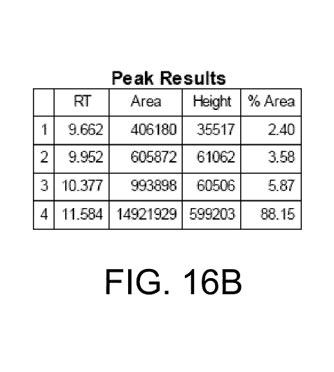

HPLC scans of the P15/Gd sample were also carried out to gauge the number and percent concentrations of different peptide species present in the sample. The buffer solutions used in the HPLC scans of P15/Gd consisted of only water and acetonitrile. The binding affinity of DO3A for Gd⁺³ ions decreases with decreasing pH, so trifluoroacetic acid (TFA) and other acidifying agents were not used in the HPLC buffers. The HPLC runs were recorded using detection wavelengths of 205 nm and 463 nm. The 205 nm detection wavelength was used to detect any π bonds present, while the 463 nm detection wavelength was used to help isolate species containing fluorescent active 5-FAM groups. The results for the 205 nm detection wavelength are shown in FIGS. 15A and 15B, and the results for the 463 nm detection wavelength are shown in FIGS. 16A and 16B.

Comparisons between the 205 nm and 463 nm HPLC spectra allow for the determination of species with and without an active fluorescent FAM group. Both detection wavelength spectra show a primary, weakly skewed, peak centered at ˜11.5 minutes. Several weaker peaks are also observed, however only those between 9-11 minutes retention time are thought to contain some form of the complete P15 peptide. HPLC peaks outside this range have weak to no absorbance at 463 nm and are probably impurities that are missing the FAM fluorescence group. The primary 463 nm peak contains >88% of the total peak area, showing that single complexed state of P15/Gd likely dominated in solution.

The presence of copper containing particles was also observed in the NEM buffered solutions presented in Example 1. The use of other Good's buffers were explored, however, the alternative buffers also experienced similar particles being formed, in the complete absence of the peptide. The use of the copper interacting TRIS buffer removed the appearance of initial salt particles, but appeared to promote the formation of large, red, amorphous, insoluble particles of the P15/Gd peptide. Although the solubility of the octarepeat region is expected to decrease with an increasing degree of copper complexation and is part of the natural functioning of the octarepeat region, the introduction of peptide-buffer interactions over the introduction buffer-metal interactions was avoided in the experimental design. The formation of large, red, amorphous insoluble particles of the P15/Gd peptide were not observed under a microscope, using 0.1 mM peptide and a buffer solution of 50 mM NEM, at pH=7.0. Exposure to 10 fold excess copper did not produce any visible signs of P15/Gd amorphous particles. As a result, NEM at pH=7.0 was selected as the buffer system.

Solutions were prepared to examine the fluorescence activity of P15/Gd, when titrated with the biologically relevant ions. Fluorescence phenomena were selected as the primary method to study the structural behavior and thermodynamics of the P15/Gd peptide, because the fluorescence intensities were assumed to be partially independent of the localized metal coordination chemistries occurring inside each ¹H MRI tag site. Solutions of 10 and 100 μM P15/Gd were titrated separately with 0-1000 μM of CaCl₂, ZnCl₂, and CuSO₄. The solutions were incubated at room temperature, in the dark, for 120 minutes, to allow equilibrium to be reached. The fluorescence was measured with a Fluoroskan FL machine. The excitation filter wavelength was 485 nm, and the fluorescence emission filter was 520 nm. The pH of each final solution was checked with pH paper to confirm that the pH of each solution remained constant.

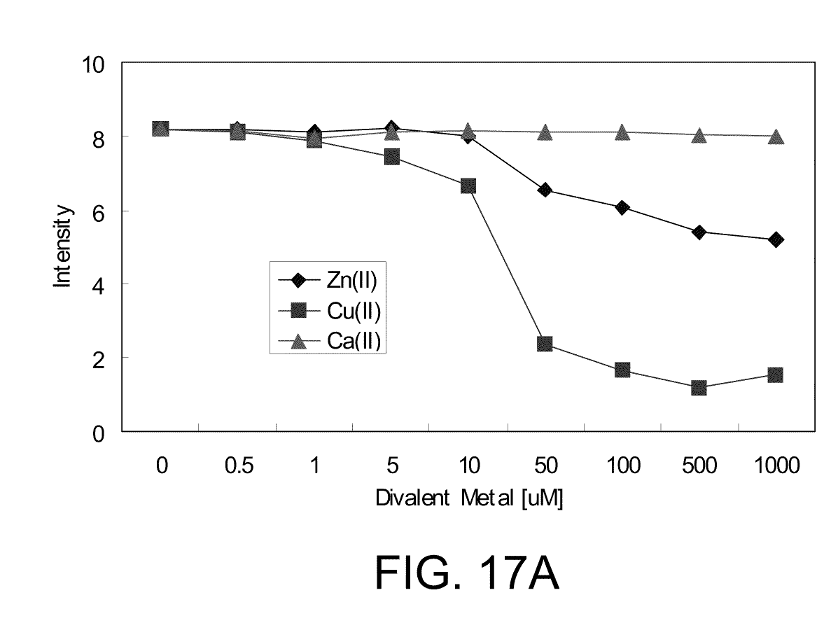

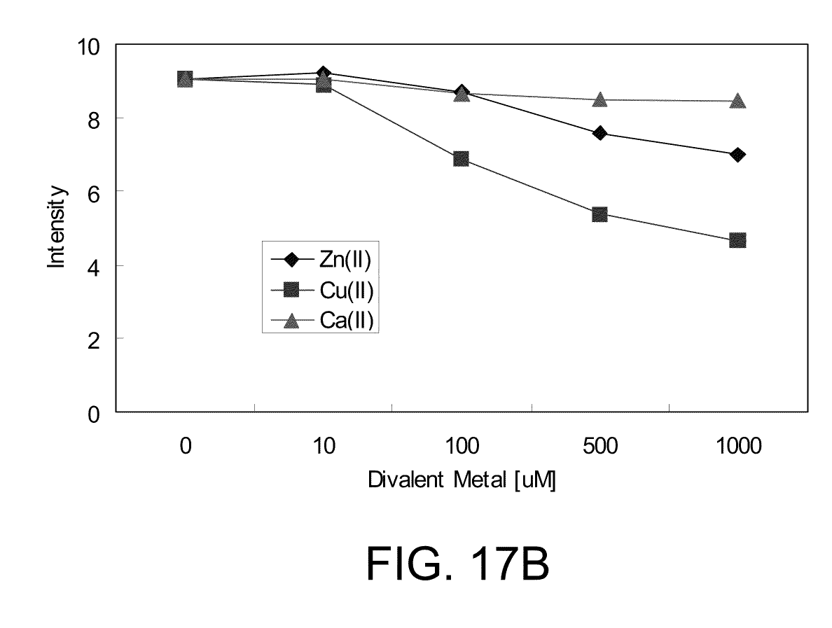

The fluorescence titration results are shown in FIGS. 17A and 17B. The binding affinity of P15/Gd with copper was the strongest, and a weaker binding affinity for zinc was observed. These fluorescence results reveal that the selectivity of P15/Gd is analogous to P1P and that P15/Gd is primarily selective towards Cu⁺² ions. Using Equation 4 and the titration data against 10 μM of P15/Gd, the dissociation constant between P15/Gd and Cu⁺² was determined to be 14.7 μM±11.1 μM, at pH=7. A very weak interaction with calcium was also observed against 100 μM P15/Gd, although the intensity shift was on the same order as the uncertainty in the experiment.

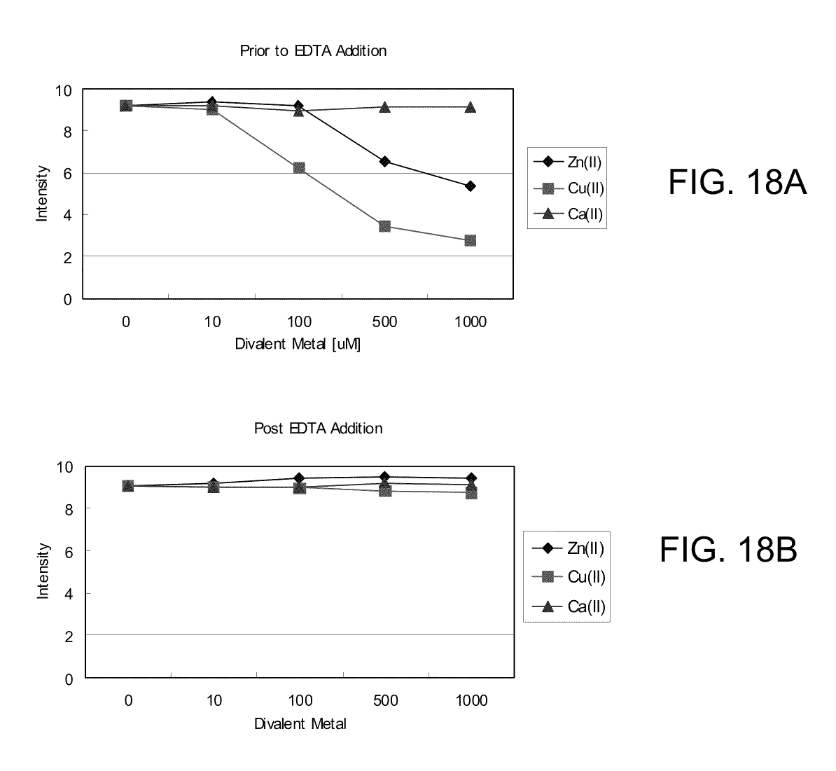

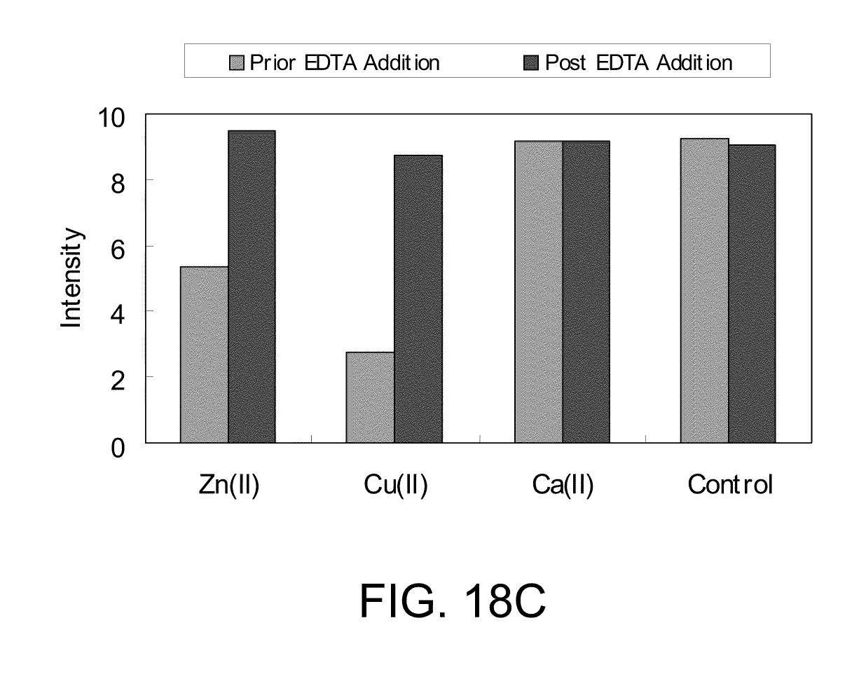

The reversibility of target binding is a key property of any smart contrast agent. The reversibility of P15/Gd was examined by treating the resultant solutions from the fluorescence titration experiments, with excess EDTA (FIGS. 18A and 18B). A concentrated solution of EDTA was added (for a total final solution concentration of 5 mM EDTA) to each of the divalent metal conditions. The pH of each solution was checked with pH paper, to assure the pH remained unchanged. FIG. 18C show the change in fluorescence intensity for each of the 1000 μM divalent metal ion conditions, before and after the EDTA treatment. In each case the fluorescence quickly returned to the fluorescence intensity of the P15/Gd only control condition, showing that reactions with copper, zinc, and/or calcium were reversible.

The r₁ relaxivity for P15/Gd, at a concentration of 100 μM, was also measured. The average value of (n_{L}r_{1,p}) was found to be ˜31.0 mM⁻¹ s⁻¹ and ˜22.5 mM⁻¹ s⁻¹, at a Larmor frequency of 0.01 MHz and 20 MHz, respectively. The relaxivity for P15/Gd was several times larger than other smart ¹H MRI contrast agents currently reported for copper. Larger relaxivity values for P15/Gd are consistent with the general prediction that compounds with higher molecular weights and larger numbers of Gd⁺³ ions coordinated sites will have higher relaxivity values. Smart contrast agents with larger relaxivity values imply lower MRI detection limits for the target species and are advantageous over contrast agents with lower relaxivity values.

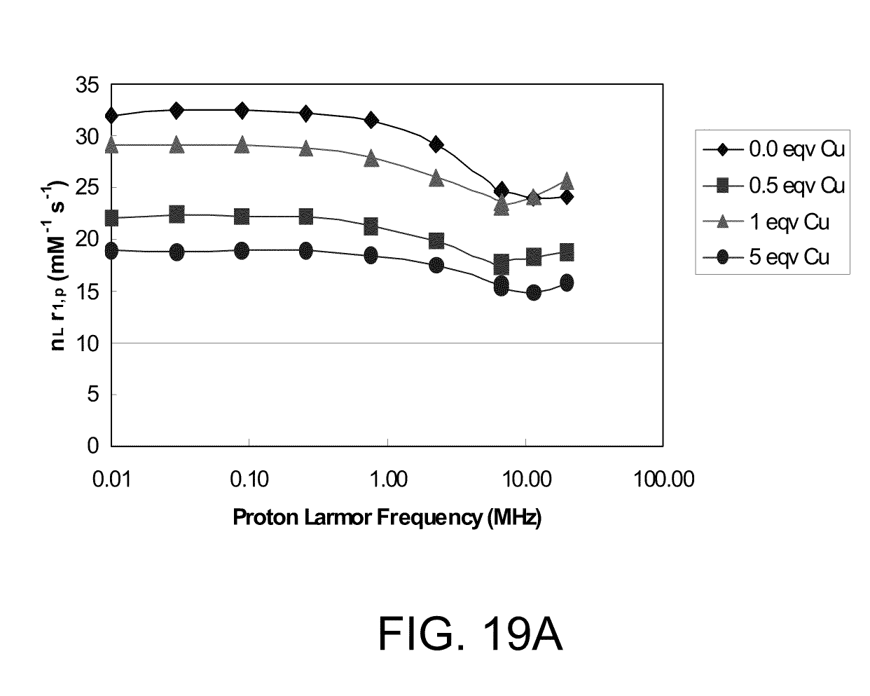

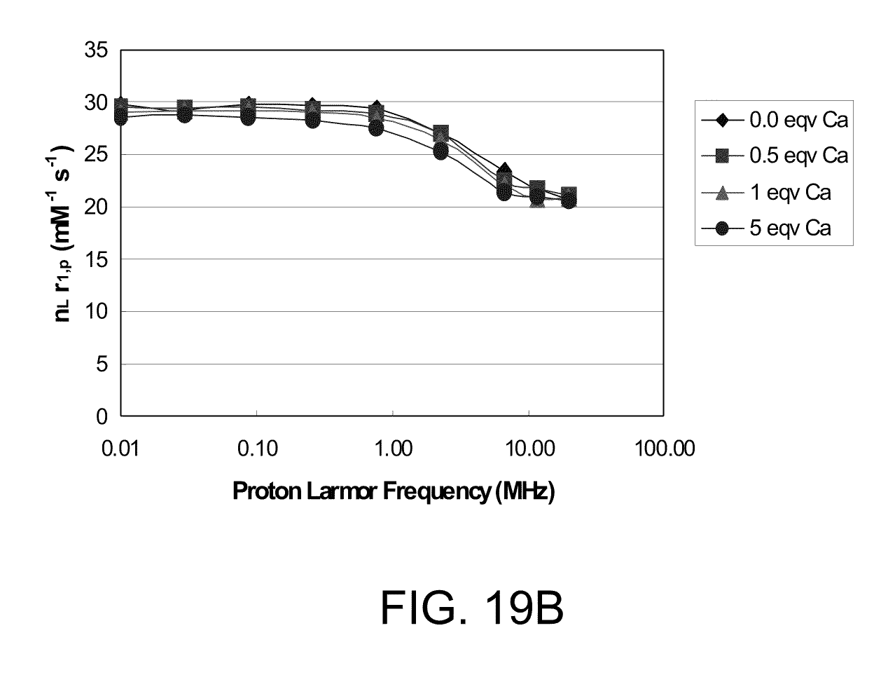

To determine the effect of copper ions on the T₁ relaxation times, 0.1 mM P15/Gd solutions in 50 mM NEM buffer, at pH=7.0, were incubated with 0.0 mM, 0.05 mM, 0.1 mM, and 0.5 mM CuSO₄. The nuclear magnetic relaxation dispersion (NMRD) profiles are given in FIG. 19A and FIG. 19B. The incubation was conducted in the dark, at room temperature, and lasted for 3 hours. The longitudinal relaxivity was measured at field strengths between 0.01 MHz to 20 MHz. The relaxivity of only CuSO₄ and GdCl₃, in 50 mM NEM, at pH=7.0, was found to be 0.4 mM⁻¹ s⁻¹ and 5.1 mM⁻¹ s⁻¹, at 20 MHz, respectively. The r₁ relaxation times exhibited a complex behavior toward the addition of Cu⁺² ions. The underlining mechanisms responsible for the observed behavior have not been well characterized to date, but likely contain contributions from several factors including gadolinium-copper ion exchange, alterations in peptide solubility, peptide structural rearrangements, remaining synthesis impurities, and buffer interactions with both copper and gadolinium ions. Similar titrations using CaCl₂ produced little change in the baseline P15/Gd solution relaxivity, which showed the P15/Gd peptide can operate as a selective MRI contrast agent for Cu⁺², against a high Ca⁺² concentration background.

### Example 3

### A Fluorescent Agent that Uses 2 Labeling Groups with Improved Metal Binding Affinities

The compound used in this example will be referred to as P41, includes the following sequence, K(6-FAM)PHGGGWGQK(dabcyl), where the K(6-FAM) is shown below.

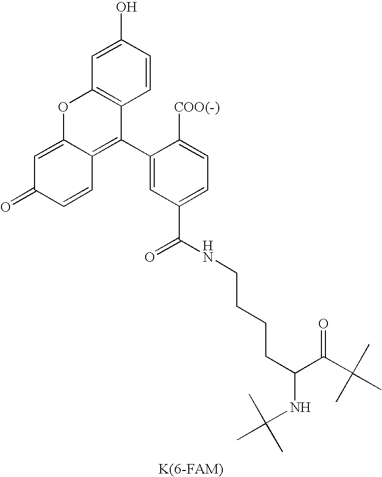

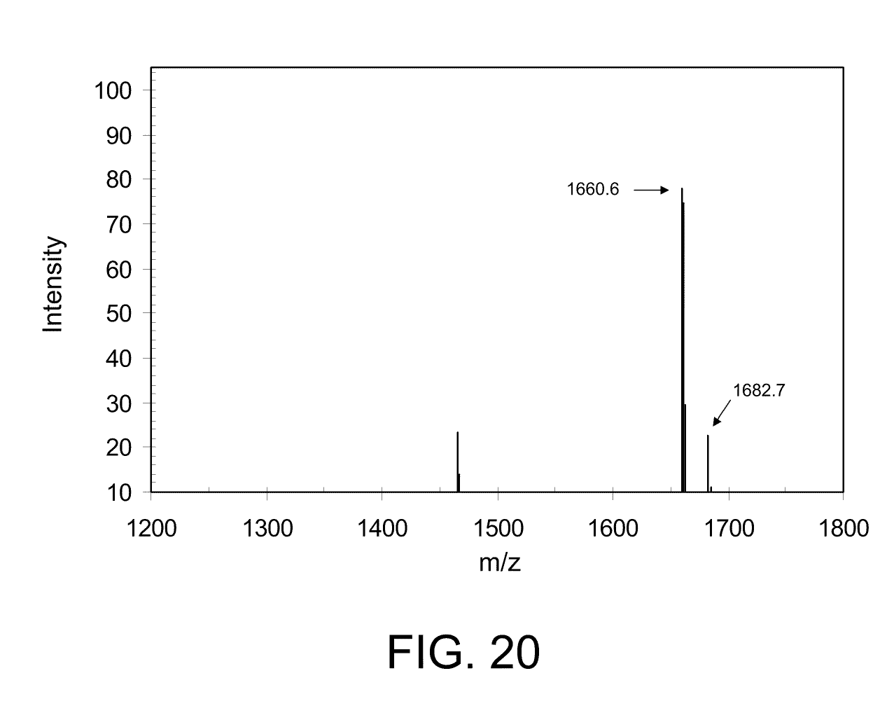

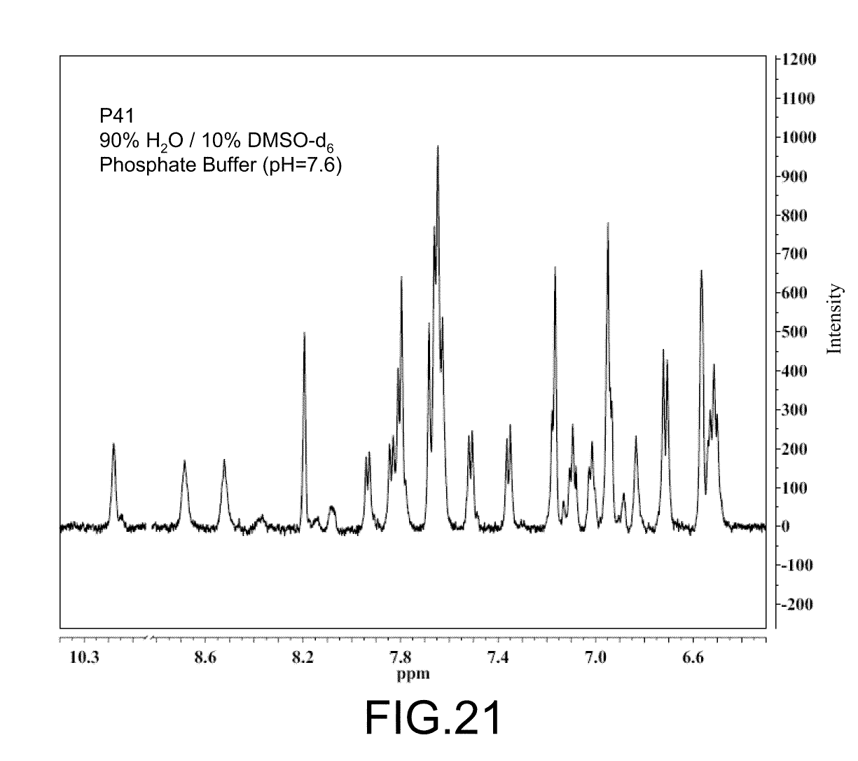

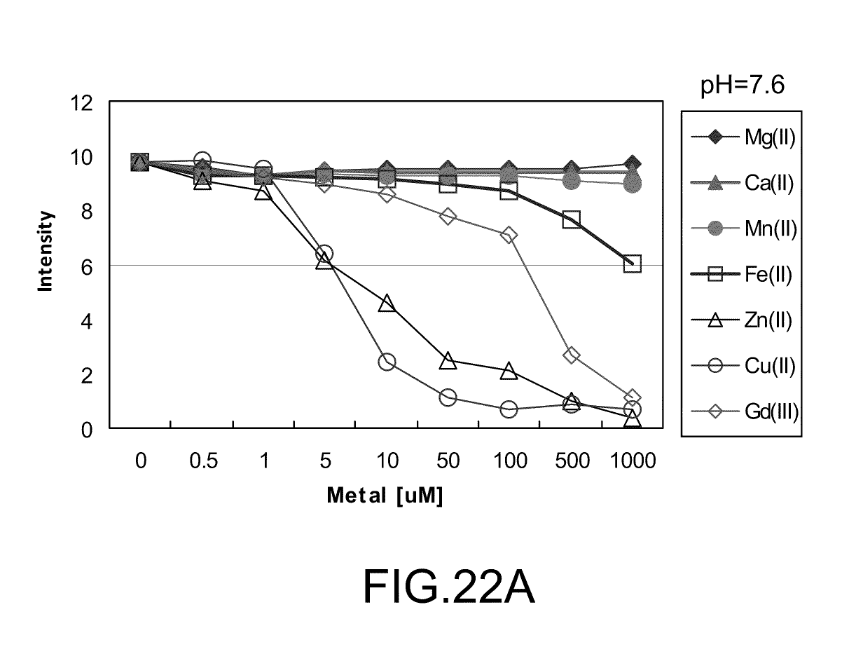

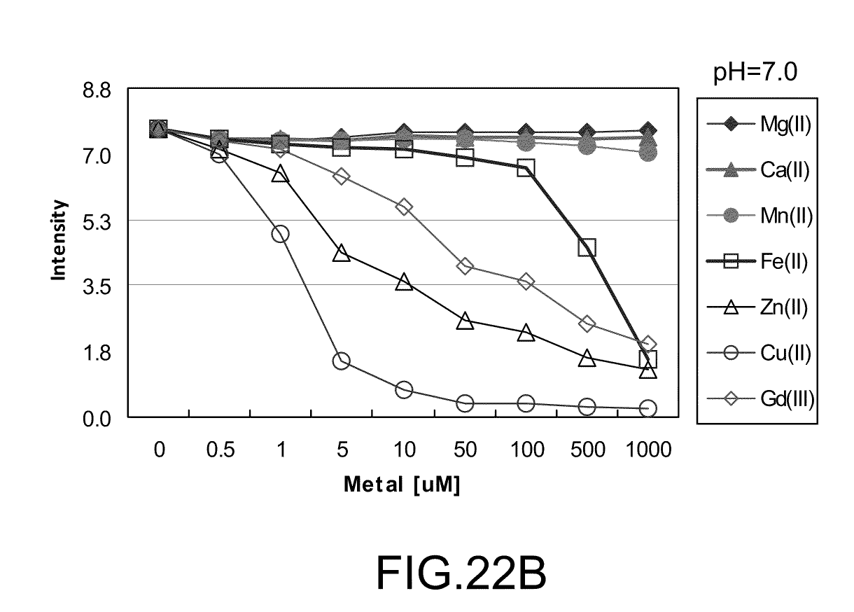

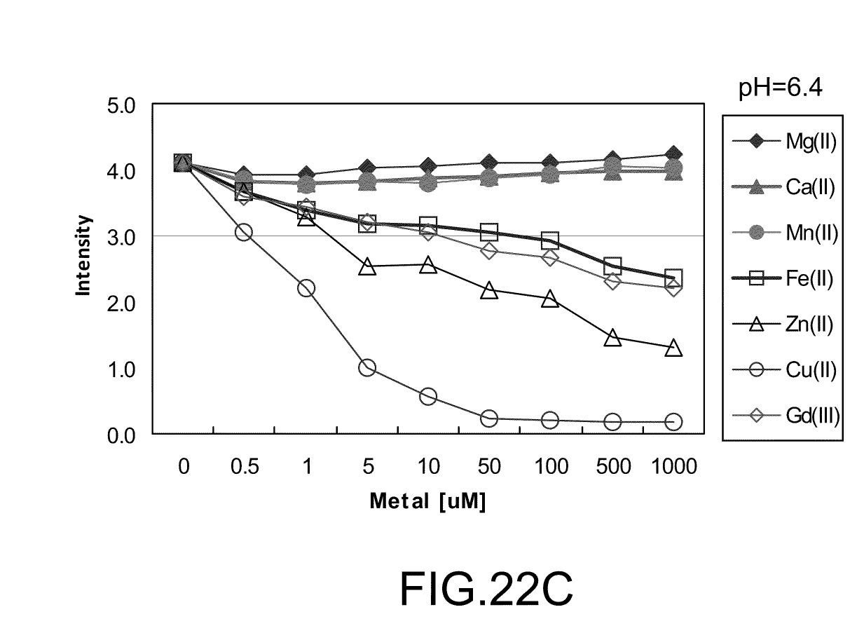

P41 was chemically synthesized, in its zwitterionic form, using standard Fmoc/tBu methods (W C Chan and PD White 2000). Commercially available Fmoc-Lys(FAM)-OH building blocks were found to sometimes suffer from lower than advertised purity and an unclear isomeric chemical structure. To achieve a higher quality and more precise synthesis, Fmoc-Lys(FAM)-OH building blocks were not used. The 6-FAM group was instead directly added to the target lysine side chain in an additional step following the primary synthesis of the peptide backbone chain. The crude peptide product was purified using reverse-phase HPLC. Purification was continued until >90% purification was achieved, as confirmed by HPLC and electrospray mass spectrometry (FIG. 20). To better characterize the chemistry of P41, a high resolution 600 MHz ¹H NMR scan of the peptide in a 90/10% H₂O/DMSO-d6 solvent mixture was conducted using a sodium phosphate buffer (pH=7.6). The ¹H NMR spectrum was obtained at room temperature, by averaging 512 individual NMR scans. The hydrophilic region of the spectrum is shown in FIG. 21.

Fluorescence titrations were used to determine the ability of P41 to bind with various cations in aqueous solutions of 10 μM P41, 20 mM buffer, and 2% DMSO. These solutions of P41 were titrated with CaCl₂, MgCl₂, MnCl₂, FeCl₂, CuSO₄, ZnCl₂, and GdCl₃ (FIGS. 21A-21C). The excitation filter wavelength was 485 nm and the fluorescence emission filter was 520 nm. The solutions were buffered at pH=6.4, pH=7.0, and pH=7.6, using either NEM or MES, as appropriate. The fluorescence of each condition was tracked over a period of 120 minutes, to insure equilibrium was obtained. After 120 minutes the pH of each condition was rechecked with pH paper, to confirm the pH remained constant during the experiment.

P41 showed no change in fluorescence following treatment with large excesses of Ca⁺² and Mg⁺², indicting P41 does not interact with these ions. Interactions with Fe⁺² were present, but were weak.

The reaction of P41 with Cu⁺² was strong at all pH values studied. The model presented in Equation 4 was found to describe the interaction of P41 with Cu⁺² and yielded binding constants under 1 μM. The NMR spectrum of P41 in a sodium phosphate buffer solution (pH=7.6) was also observed during the titration of 0.05, 0.10, 0.25 and 0.50 molar equivalents of copper(II) ions. The titrations show a strong, selective, peak broadening of the histidine hydrogens, along with the complete destruction of the H(δ2) and H(ε1) histidine peaks following treatment with 0.05 molar equivalents of copper. This is consistent with the general mechanism of octarepeat copper binding. The chemical shifts of the 6-FAM and dabcyl hydrogen atoms were also observed to exhibit an upward field shift during the copper titration. This indicated that the electrostatic environment of the donor and acceptor labeling group becomes more hydrophobic with copper binding and may indicate an increased association between the FAM and dabcyl groups.

The stability of P41 inside the body is also an important factor determining the practical usefulness of the current invention. It is possible that proteins and enzymes inside the body could adversely react with P41, causing a premature degradation of the peptide. To test the practically of using P41 in vivo, solutions of P41 in fetal bovine serum and P41 in a control protein free phosphate buffer solution were prepared. The solutions were then incubated at 37° C. in the dark for a period of 2 hours. The 2 hour incubation condition is a strong test of the compound's blood stability because imaging scans are usually completed within 30 minutes following the injection of a contrast agent.

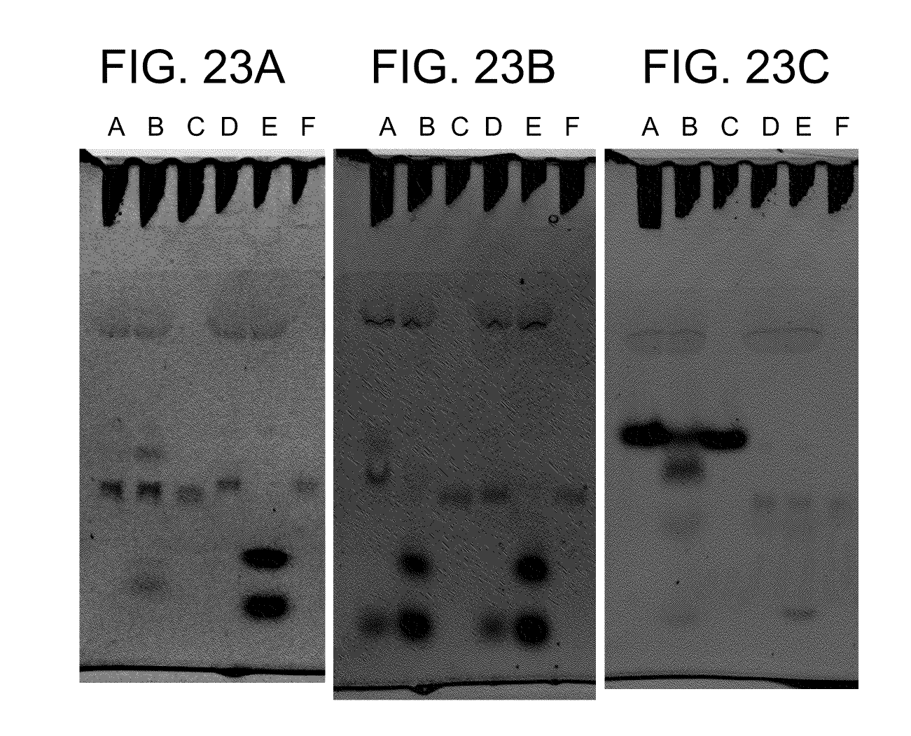

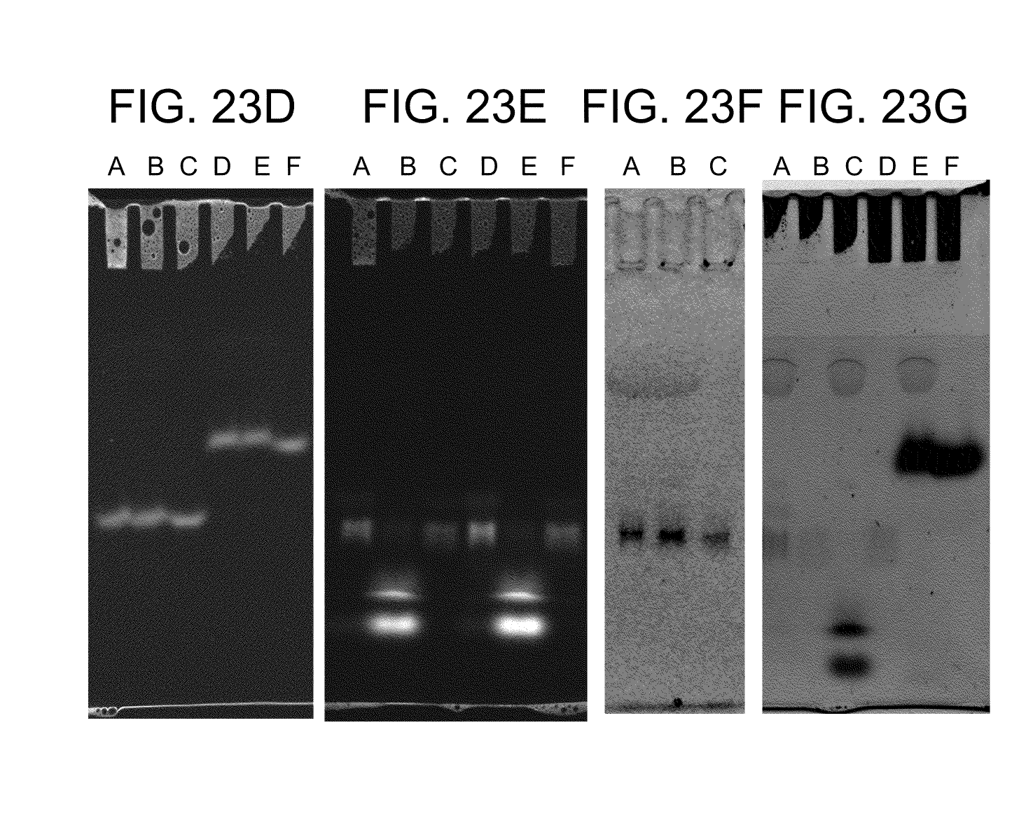

As shown in FIG. 23A, an SDS-PAGE procedure was performed to check if any fragmentation bands were present, thereby indicating that a degradation reaction was occurring in animal serum. The labeled columns denote the following: A, P41 exposed to serum for 0 hours; B, P41 exposed to serum for 2 hours; C, P41 in a control phosphate buffer; D, P42 exposed to serum for 0 hours; E, P42 exposed to serum for 2 hours; and F, P42 in a control phosphate buffer. For the condition of 0 hour exposure, the peptide samples were mixed with serum at room temperature, but then treated with an enzyme stop solution within 2 minutes of exposure. Any fragmentation of either the P41 or P42 peptide chain causes a significant increase in the fluorescence intensity because the dabcyl group is no longer able to quench the FAM group. This property makes the test highly sensitive for peptides containing both a FAM and a dabycl group. Only a very minor fragmentation of P41 was detected after 2 hours of serum incubation. P42 was not stable and was completely cut by enzymes inside the blood serum.

The high stability of P41 is advantageous over other similar peptide sequences that are not as resistant to enzyme attack. The results of the same test for other peptide sequences, according to aspects of the present teachings, are included in FIGS. 23B-23G as a comparison.

FIG. 23B illustrates the bovine serum stability of the P45 and P46 peptides. The labeled columns denote the following: A, P45 exposed to serum for 0 hours; B, P45 exposed to serum for 2 hours; C, P45 in a control phosphate buffer; D, P46 exposed to serum for 0 hours; E, P46 exposed to serum for 2 hours; and F, P46 in a control phosphate buffer.

FIG. 23C illustrates the bovine serum stability of the P58 and P48 peptides. The labeled columns denote the following: A, P58 exposed to serum for 0 hours; B, P58 exposed to serum for 2 hours; C, P58 in a control phosphate buffer; D, P48 exposed to serum for 0 hours; E, P48 exposed to serum for 2 hours; and F, P48 in a control phosphate buffer.

FIG. 23D illustrates the bovine serum stability of the P57 and P57/Gd peptides. The labeled columns denote the following: A, P57 exposed to serum for 0 hours; B, P57 exposed to serum for 2 hours; C, P57 in a control phosphate buffer; D, P57/Gd exposed to serum for 0 hours; E, P57/Gd exposed to serum for 2 hours; and F, P57/Gd in a control phosphate buffer.

FIG. 23E illustrates the bovine serum stability of the P1S and P1P peptides. The labeled columns denote the following: A, P1S exposed to serum for 0 hours; B, P1S exposed to serum for 2 hours; C, P1S in a control phosphate buffer; D, P1P exposed to serum for 0 hours; E, P1P exposed to serum for 2 hours; and F, P1P in a control phosphate buffer.

FIG. 23F illustrates the bovine serum stability of the P31 peptide. The labeled columns denote the following: A, P31 exposed to serum for 0 hours; B, P31 exposed to serum for 2 hours; C, P31 in a control phosphate buffer.

FIG. 23G illustrates the bovine serum stability of the P43, P44, and P50 peptides. The labeled columns denote the following: A, P43 exposed to serum for 2 hours; B, P43 in a control phosphate buffer; C, P44 exposed to serum for 2 hours; D, P44 in a control phosphate buffer; E, P50 exposed to serum for 2 hours; F, P50 in a control phosphate buffer.

The P42, P44, P45, P48, and P1S peptides were found to be strongly fragmented. The peptide P58, which does not contain a labeling group at the N-terminus, was also found to be significantly fragmented. Other peptide sequences, including P1P, P43, P50, P54, P57, and

P57/Gd, were not fragmented. These experiments showed that one key area affecting the stability of the peptides in animal serum is the appropriate selection of the N-terminus labeling group chemistry. Enhanced stability in serum was also obtained, as evidenced by P43, by substituting D-proline in place of the leading L-proline residue of the core peptide sequence.

### Example 4

### A Highly Copper Selective Agent that Uses Both a Fluorescent and Fluorine Labeled Group

The compound used in this example will be referred to as P50 and includes the following sequence, Ac-K(6-FAM)PHGGGWGQP(trifluoromethyl)NH₂, where the P(trifluoromethyl) is describe below.

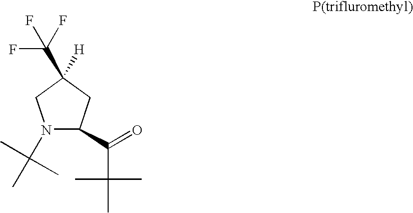

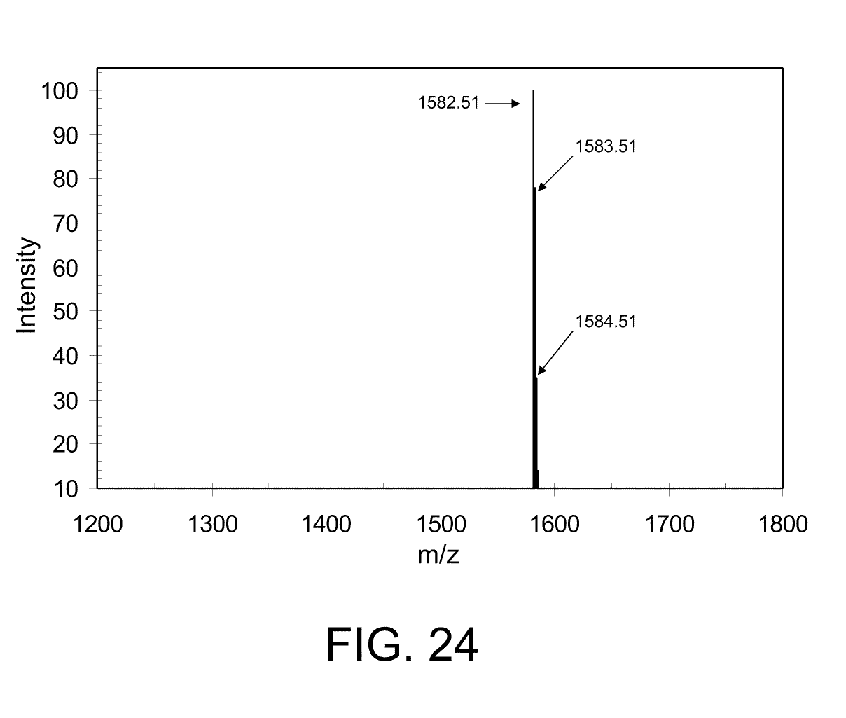

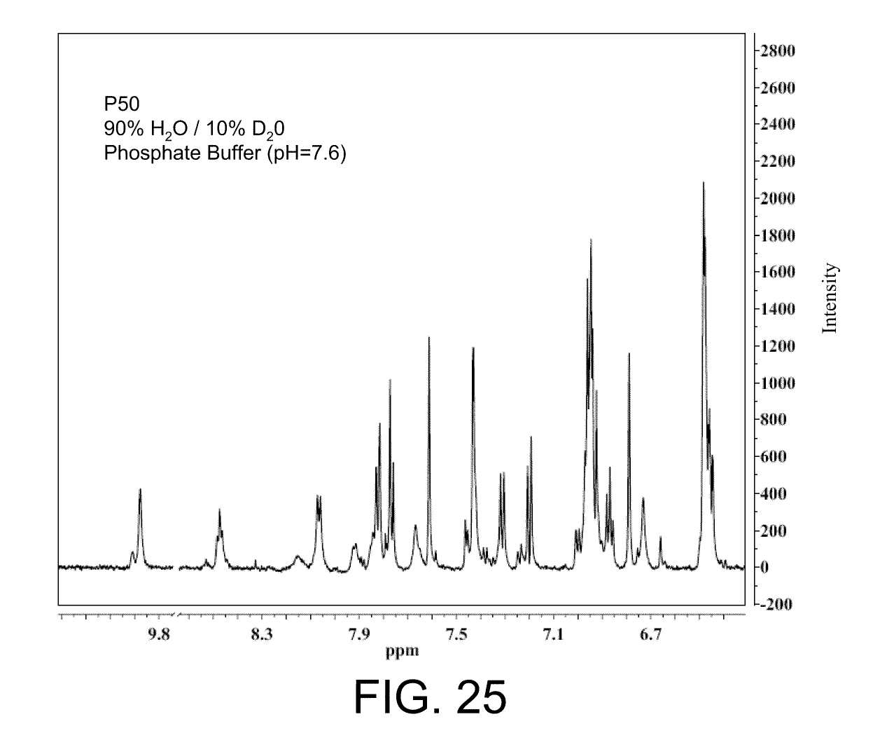

P50 was chemically synthesized, in its neutral free amine-acetylated form, using standard Fmoc/tBu methods (W C Chan and PD White 2000). Commercially available Fmoc-Lys(FAM)-OH building blocks were found to sometimes suffer from lower than advertised purity and unclear isomeric chemical structures. To achieve a higher quality and more precise synthesis, Fmoc-Lys(FAM)-OH building blocks were not used. The 6-FAM group was instead directly added to the target lysine side chain, in an additional step following the primary synthesis of the peptide backbone chain. The P(trifluoro) group was added using a commercially available Fmoc-Pro(trifluoro)-OH building block. The electrospray mass spectrum of P50 is shown in FIG. 24. To better characterize the chemistry of P50, a high resolution 600 MHz ¹H NMR scan of the peptide in a 90/10% H₂O/D₂O sodium phosphate buffered (pH=7.6) solvent mixture was conducted. The ¹H NMR spectrum was obtained, at room temperature, by averaging 512 individual NMR scans. The hydrophilic region of the spectrum is shown in FIG. 25.

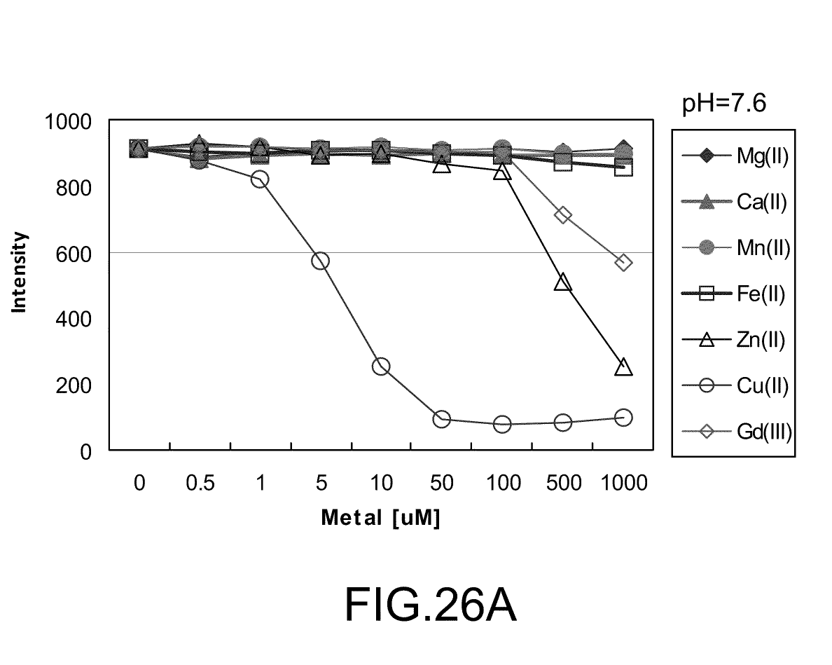

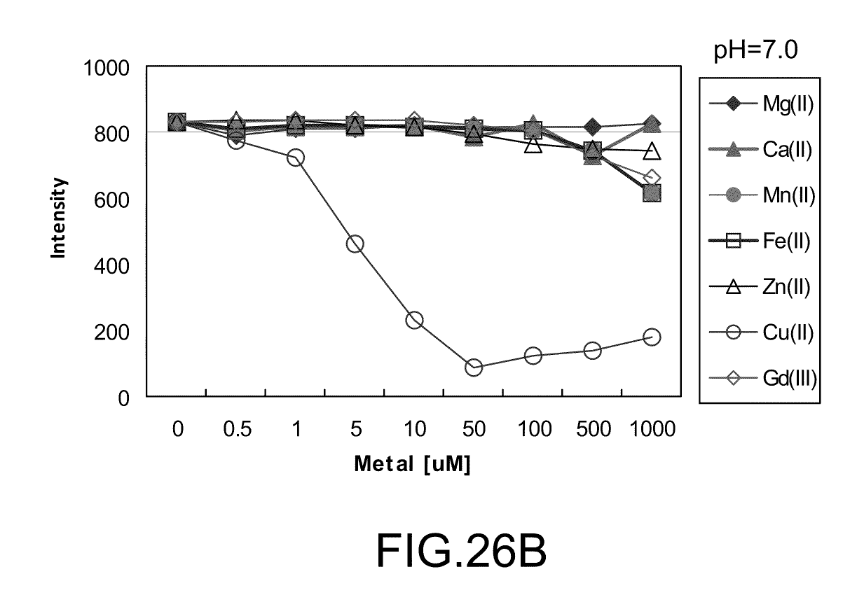

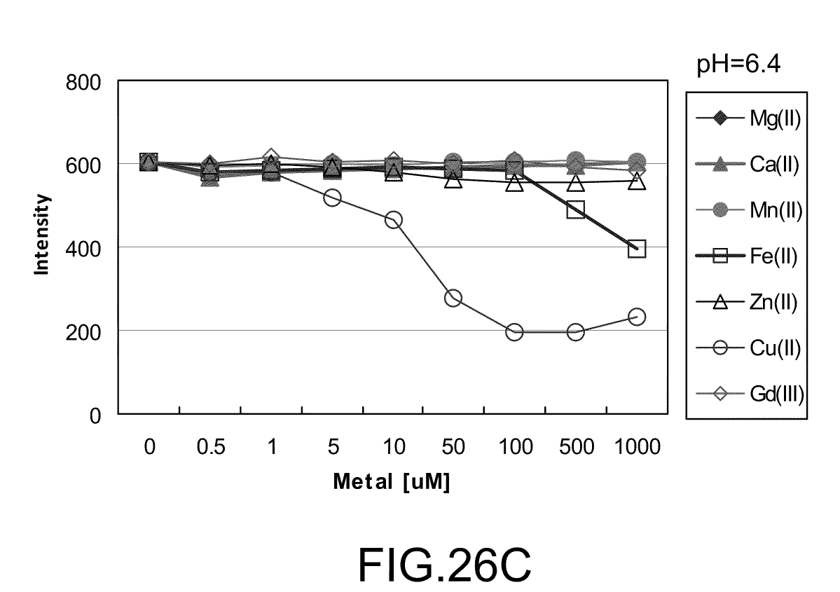

Fluorescence titrations were used to determine the ability of P50 to bind with various cations, in aqueous solutions of 10 μM P50 and 20 mM buffer. These solutions of P50 were titrated with CaCl₂, MgCl₂, MnCl₂, FeCl₂, CuSO₄, ZnCl₂, and GdCl₃ (FIGS. 26A-26C). The excitation filter wavelength was 485 nm, and the fluorescence emission filter was 520 nm. The solutions were buffered at pH=6.4, pH=7.0, and pH=7.6 using either NEM or MES, as appropriate. The fluorescence of each condition was tracked over a period of 120 minutes, to insure equilibrium was obtained. After 120 minutes, the pH of each condition was rechecked with pH paper, to confirm the pH remained constant during the experiment.

P50 showed no fluorescence change following treatment with large excesses of Ca⁺² and Mg⁺², indicting the P50 does not interact with these ions. Interactions with Zn⁺² and Fe⁺² were present, but were weak.

The reaction with P50 with Cu⁺² was strong at all pH values studied. The selectivity of P50 for Cu⁺² was significantly higher than that of P1P, P15, P20, and P41. The fluorescence intensity was also significantly larger, because of the lack of the dabcyl quenching group. The model presented in Equation 4 was found to describe the interaction of P50 with Cu⁺² and yielded binding constants of 19.1 μM±8.3 μM, 0.49 μM±0.4 μM, and 0.42 μM±0.9 μM, at pH 6.4, 7.0, and 7.6, respectively. The NMR spectrum of P50 buffered in sodium phosphate (pH=7.6) was observed during the titration of 0.05, 0.10, 0.25 and 0.50 molar equivalents of copper(II) ions. The titrations show a strong, selective peak broadening of the histidine hydrogens, along with a complete destruction of H(δ2), H(ε1), and H(β) histidine peaks following treatment with 0.25 molar equivalents of copper. This is consistent with the general mechanism of octarepeat copper binding. No significant alterations of the chemical shifts of the 6-FAM hydrogen atoms were observed, which indicates that electrostatic environment of the 6-FAM labeling group is not significantly affected when P50 binds (up to 0.50 molar equivalents) of copper.

The strong binding affinity of Cu⁺² ions with P50 further highlights its application as a therapeutic copper chelating agent. P50 was tested in vivo using adult male ICR mice to determine its pharmacokinetics (Table 7). The peptide was delivered using a 200 μL tail vein injection. Two doses of P50 were examined. The first dose was 2 mM P50 in 200 μL of physiological saline. The second, higher dose was 8 mM P50 in 200 μL of physiological saline. No adverse toxicological reactions were observed in the mice following the injections. The mice were sacrificed at 5, 10, and 15 minutes post injection, and their serum and internal organs were harvested. The brain, kidney, and liver tissues were homogenized prior to analysis. The fluorescence intensity of P50 was quite strong and was easily detected in all samples using fluorescence RP-HPLC. The RP-HPLC conditions were as follows: solution A, 5 mM phosphate buffer (pH=7.6); solution B, 5 mM phosphate buffer (pH=7.6) and 65% acetonitrile; column, 4.6×250 mm Cosmosil 5C18-SR-II; detector settings, excitation wavelength=490 nm and emission wavelength=525 nm. The RP-HPLC peaks detected in the serum samples were then purified and confirmed by SDS-PAGE and electrospray mass spectrum to be the P50 peptide.

TABLE 7
Mouse Pharmacokinetics of P50

| | Post Injection | | | |
|---|---|---|---|---|
| Dose | Time (min) | Tissue/Fluid | [P50] | Blood Ratio |
| 2 mM | 5 | Serum | 128 μg/mL | — |
| | 10 | | 116 μg/mL | — |
| | 15 | | 62 μg/mL | — |
| | 5 | Brain | 1225 ng/g | 1.0% |
| | 10 | | 1197 ng/g | 1.0% |
| | 15 | | 782 ng/g | 1.3% |
| | 5 | Liver | 10.2 μg/g | 8.0% |
| | 10 | | 9.4 μg/g | 8.1% |
| | 15 | | 3.8 μg/g | 6.1% |
| | 5 | Kidney | 34 μg/g | 26% |
| | 10 | | 27 μg/g | 23% |
| | 15 | | 18 μg/g | 29% |
| 8 mM | 5 | Serum | 236 μg/mL | — |
| | 10 | | 235 μg/mL | — |
| | 15 | | 227 μg/mL | — |
| | 5 | Brain | 4477 ng/g | 1.9% |
| | 10 | | 4242 ng/g | 1.8% |
| | 15 | | 2757 ng/g | 1.2% |
| | 5 | Liver | 38 μg/g | 16% |
| | 10 | | 30 μg/g | 13% |
| | 15 | | 25 μg/g | 11% |
| | 5 | Kidney | 85 μg/g | 36% |
| | 10 | | 123 μg/g | 52% |
| | 15 | | 70 μg/g | 30% |

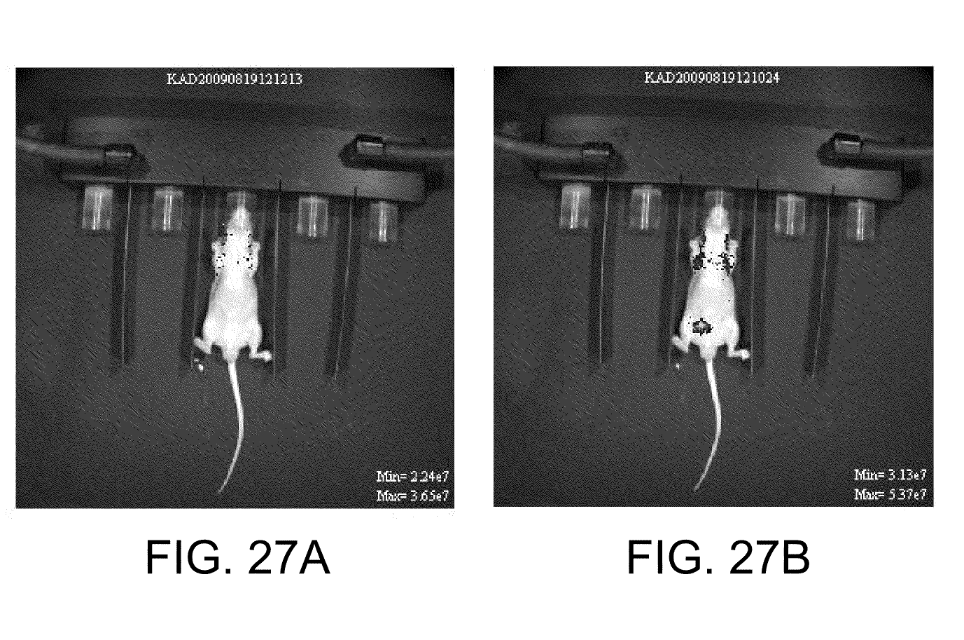

FIGS. 27A-27B illustrate fluorescence images of the approximate position of P50 inside a nude mouse(Jackson Laboratory strain 2019), using respective excitation pulses of 430 and 460 nm after 20 minutes, following a 2 mM P50×200 μL tail vein injection. The mouse was fed on an alfalfa free diet for one week to reduce any auto fluorescence from partially digested food. Immediately following the injection the mouse was anesthetized using isofluorane. The images were obtained using a Xeongen fluorescence imaging system. FIG. 27A shows the fluorescence image through a GFP emission filter following an excitation pulse at 430 nm. This image is a control image since the 6-FAM group of the P50 peptide is not strongly excited by a 430 nm pulse. FIG. 27B shows the fluorescence image through a GFP emission filter following an excitation pulse at 460 nm, which however does strongly excitation of the 6-FAM group. A significant emission signal is clearly visible in the lower abdominal region of the mouse and is most likely the result of accumulation of P50 inside bladder.

The amount of P50 found inside the brain increased proportionally with injection dose, indicating that the amount of P50 inside the brain had not yet reached a saturated level. The average amount of P50 inside the brain was estimated to be 0.6 μM and 2.4 μM for the 2 mM and 8 mM injection doses, respectively. These concentrations are comparable to the amount of liable copper present in brain tissue.

An additional channel for reporting on the targeted metal is also made possible by the attachment of the fluorine label. This permits the detection of P50 using ¹⁹F MRI techniques. The P50 peptide experiences a change in its net charge during copper binding. The positive increase in net charge alters the solubility and transportation kinetics of P50, altering the pharmacokinetic profile of the smart contrast agent during copper binding. The accumulation and clearance of the smart contrast agent can then be used to construct a digital map of localized copper(II) ion concentrations.

In an additional embodiment of the current invention, the P50 peptide could also be modified to function as a PET agent. The Fmoc-Pro(trifluoro)-OH residue could be replaced with Fmoc-Pro(cis-4-fluoro)-OH [Fmoc-(2S,4S)-4-fluoro-pyrrolidine-2-carboxylic acid] or Fmoc-Pro(trans-4-fluoro)-OH [Fmoc-(2S,4R)-4-fluoro-pyrrolidine-2-carboxylic acid]. These building blocks are available commercially. PET active versions incorporating ¹⁹F atoms into the fluorine site, by halogenation of the 3,4 carbon double bond in 3,4-dehydro-proline, are also contemplated. This isotope substitution is an advantageous additional embodiment of the present invention because PET imaging can detect lower contrast agent concentrations than MRI.

### Example 5

### An Agent that Uses Two Labeling Groups and Two Octarepeat Sequences

The compound used in this example will be referred to as P20 and includes the following sequence, Ac-K(5-FAM)PHGGGWGQPHGGGWGQK(dabcyl)-NH₂.

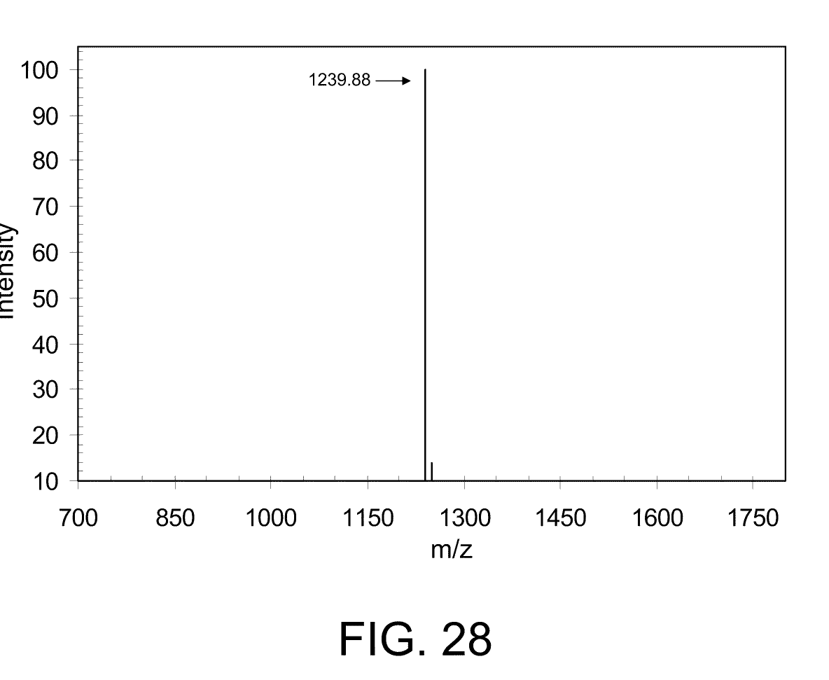

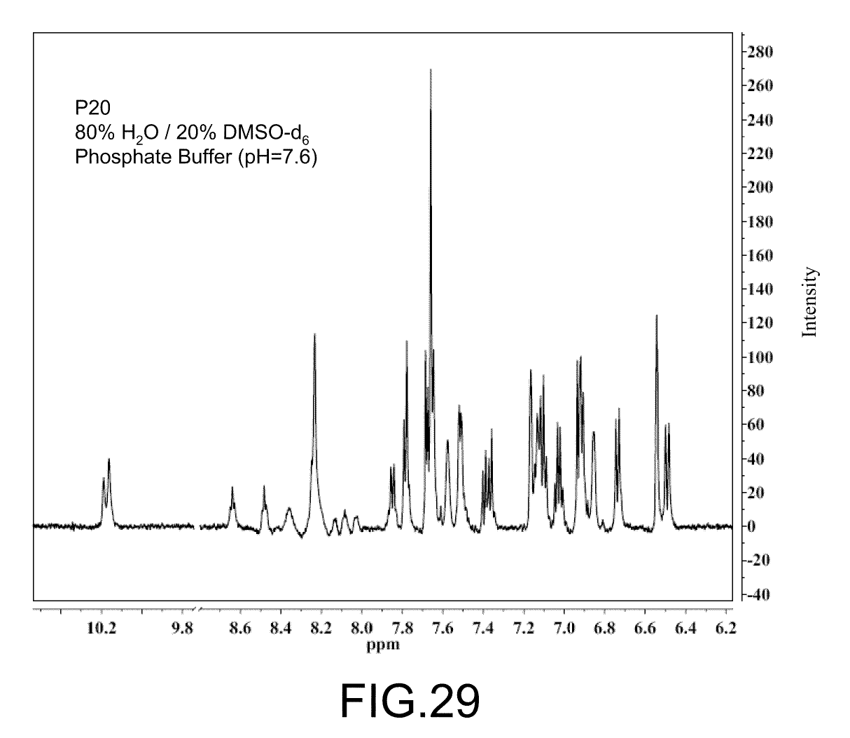

P20 was chemically synthesized, in its neutral free amine-acetylated form, using standard Fmoc/tBu methods (W C Chan and PD White 2000). The K(5-FAM) and K(dabcyl) groups were attached to the side chain of each lysine residue, prior to their attachment to the core peptide. Lysine peptide building blocks incorporating 5-FAM and dabcyl on to the side chain are commercially available as Fmoc-Lys(5-FAM)-OH and Fmoc-L-Lys(dabcyl)-OH. These building blocks were used in the synthesis procedure, using standard Fmoc/tBu techniques for peptide synthesis. The crude peptide product was purified using reverse-phase HPLC and a C18 column. Purification was continued until >95% purification, as confirmed by HPLC and electrospray mass spectrometry. The electrospray mass spectrum of P20 is shown in FIG. 28. To better characterize the chemistry of P20, a high resolution 600 MHz ¹H NMR scan of the peptide in an 80/20% H₂O/DMSO-d₆ sodium phosphate buffered (pH=7.6) solvent mixture was conducted. The ¹H NMR spectrum was obtained at room temperature, by averaging 512 individual NMR scans. The hydrophilic region of the spectrum is shown in FIG. 29.

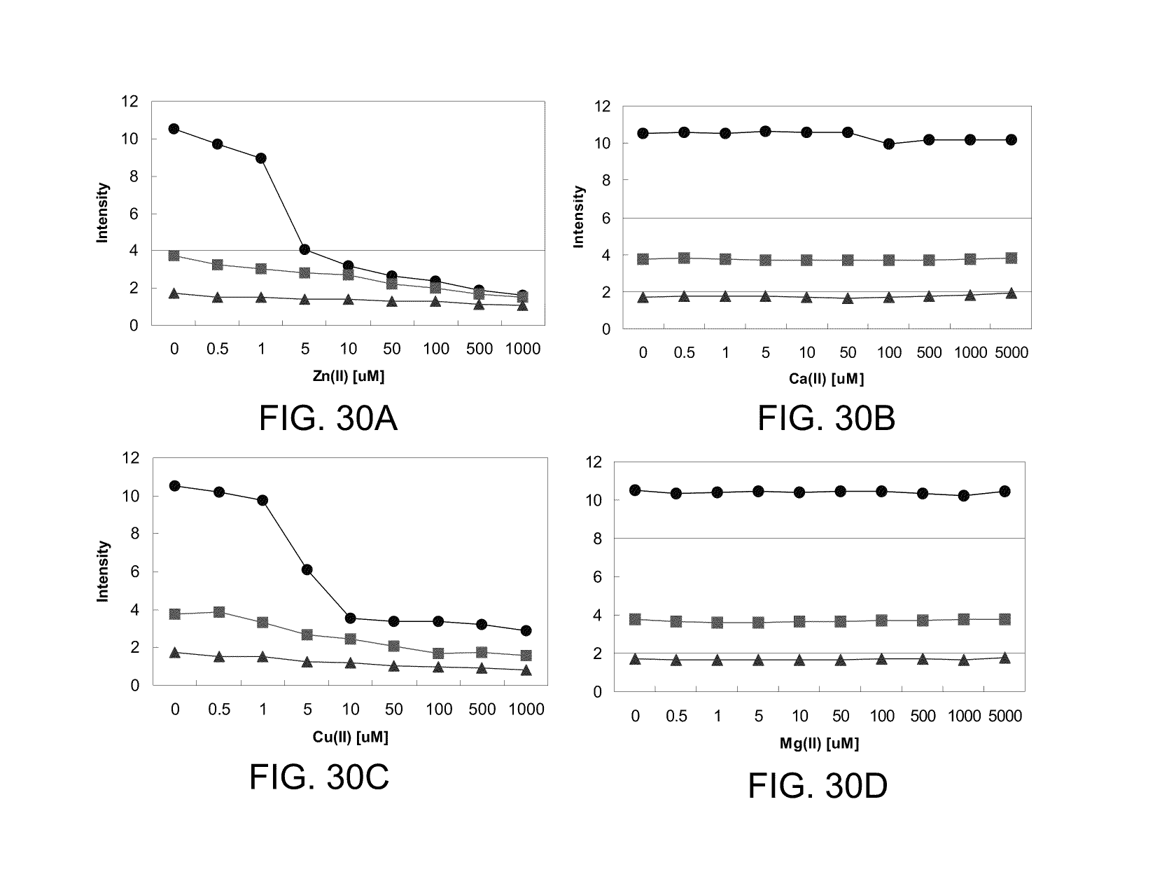

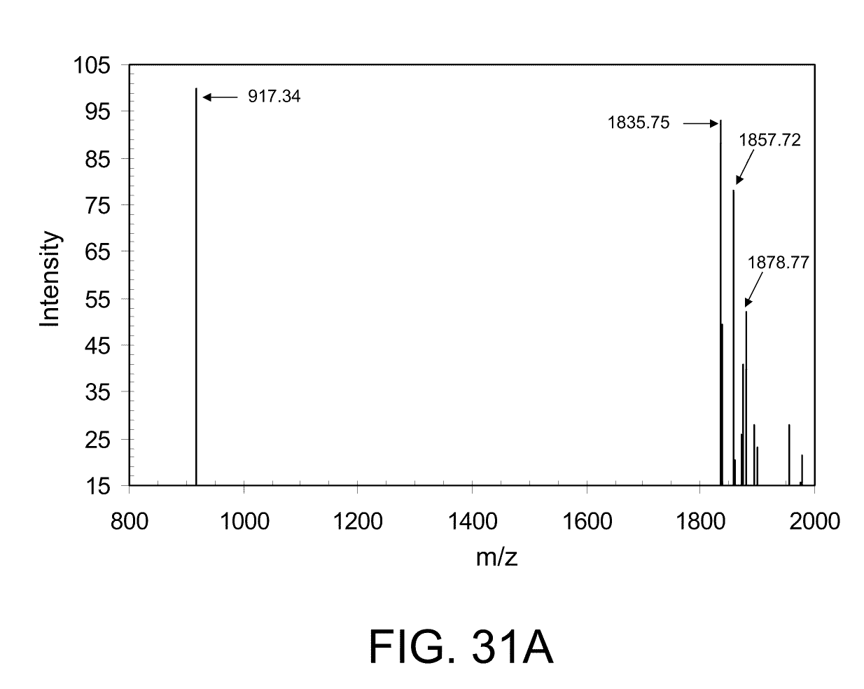

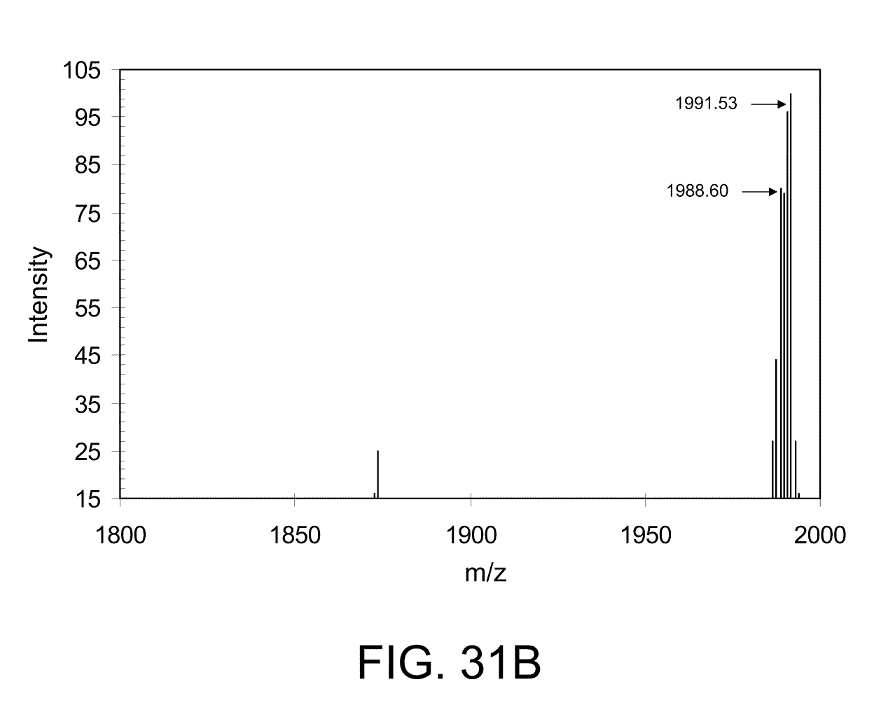

Fluorescence titrations were used to determine the ability of P20 to bind with various cations in aqueous solutions of 10 μM P20, 20 mM buffer, and 2% DMSO. These solutions of P20 were titrated with CaCl₂, MgCl₂, CuSO₄, and ZnCl₂ (FIGS. 30A-30C). The excitation filter wavelength was 485 nm, and the fluorescence emission filter was 520 nm. The solutions were buffered at pH=6.4, pH=7.0, and pH=7.6, using either NEM or MES, as appropriate. The fluorescence of each condition was tracked over a period of 120 minutes, to insure equilibrium was obtained. After 120 minutes the pH of each condition was rechecked with pH paper, to confirm the pH remained constant during the experiment.

P20 had no fluorescence change following the treatment with large excesses of Ca⁺² and Mg⁺², indicting the P20 does not interact with these ions.

The reaction with P20 with both Cu⁺² and Zn⁺² was strong at pH=7.6. The model presented in Equation 4 was found to yield binding constants for P20 and Cu⁺² of 0.017 μM±0.057 μM at pH=7.6. A similar strong reaction at pH=7.6 with Zn⁺² was also measured to be 0.08 μM+/−0.88 μM. Reactions at pH=7.0 and pH=6.4 were less well defined.

### Example 6

### A Highly Selective Agent for Copper that Uses Both a Fluorescent and DO3A Labeling Group

The compound used in this example will be referred to as P57 and includes the following sequence, Ac-K(6-FAM)PHGGGWGQK(DO3A)NH₂

P57 was chemically synthesized, in its neutral free amine-acetylated form, using standard Fmoc/tBu methods (W C Chan and PD White 2000). Commercially available Fmoc-Lys(FAM)-OH building blocks were found to sometimes suffer from lower than advertised purity and unclear isomeric chemical structures. To achieve a higher quality and more precise synthesis, Fmoc-Lys(FAM)-OH building blocks were not used. The 6-FAM group was instead directly added to the target lysine side chain in an additional step, following the primary synthesis of the peptide backbone chain. The attachment of the K(DO3A) into the peptide sequence was performed using the commercially available Fmoc-L-Lys(DO3A)-tris(t-Bu)—OH building block.

The complexation of Gd to the P57 was achieved by mixing 120 μL of 1 mM P57 with a 120 μL of 3 mM of GdCl₃. A three fold excess of Gd⁺³ ions was used to drive the complexation reaction toward completion. The pH of the mixture was adjusted to ˜5.0 units, using step wise additions of 0.01-0.1 M NaOH. The pH target of 5.0 was selected to minimize unwanted Gd salting out reactions with anionic impurities, while still favoring a strong chemical reaction of Gd⁺³ with the DO3A group. The complexation solution was then allowed to incubate in the dark at 30° C., for 2 hours. After incubation, 60 μL of 10 mM of sodium phosphate buffer (pH=8.0) was added. Phosphate and Gd⁺³ ions form an insoluble salt under neutral to basic conditions. Any Gd⁺³ ions that were not already strongly complexed by P57 were driven to react with excess PO₄⁻² ions and precipitated out of the solution. The solution was then centrifuged at 500 g, for 10 minutes, and the supernatant collected. The centrifuge step forced any suspended particles of Gd₂(PO₄)₃ out of solution, leaving only the soluble P57/Gd complexes in solution. The attachment of the strongly fluorescence 6-FAM group allowed the detection of very small amounts of P57 in a SDS-PAGE analysis. The band positions of P57 and P57/Gd in an SDS-PAGE analysis were also found to be quite different. Quality control experiments, following this complexation procedure, showed that P57 was completely reacted with Gd and produced a single P57/Gd species in solution.

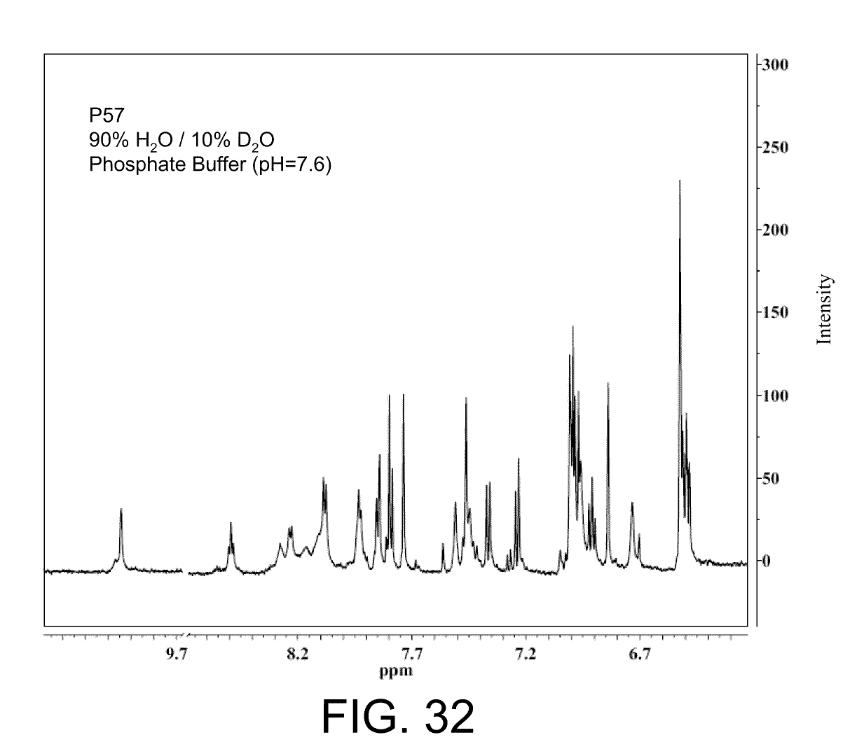

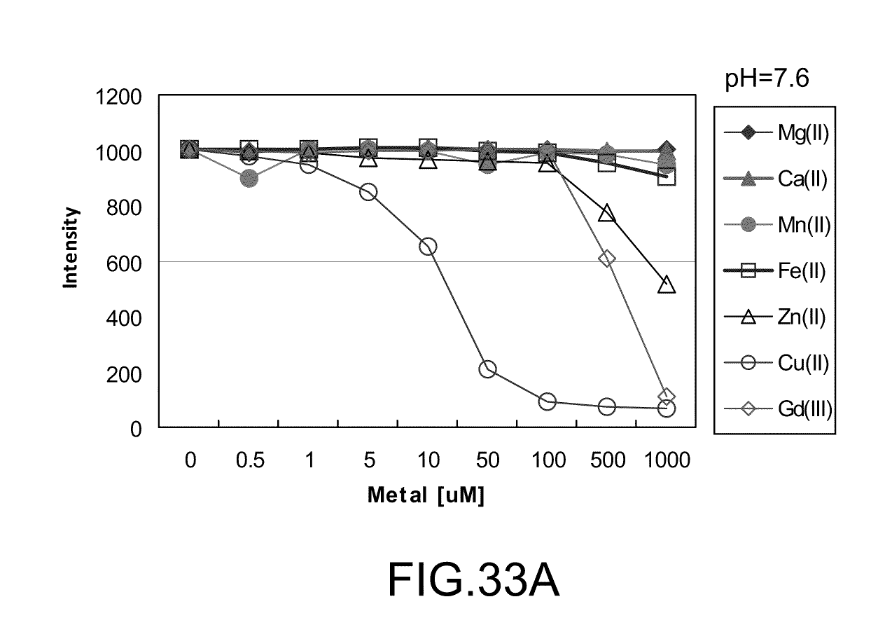

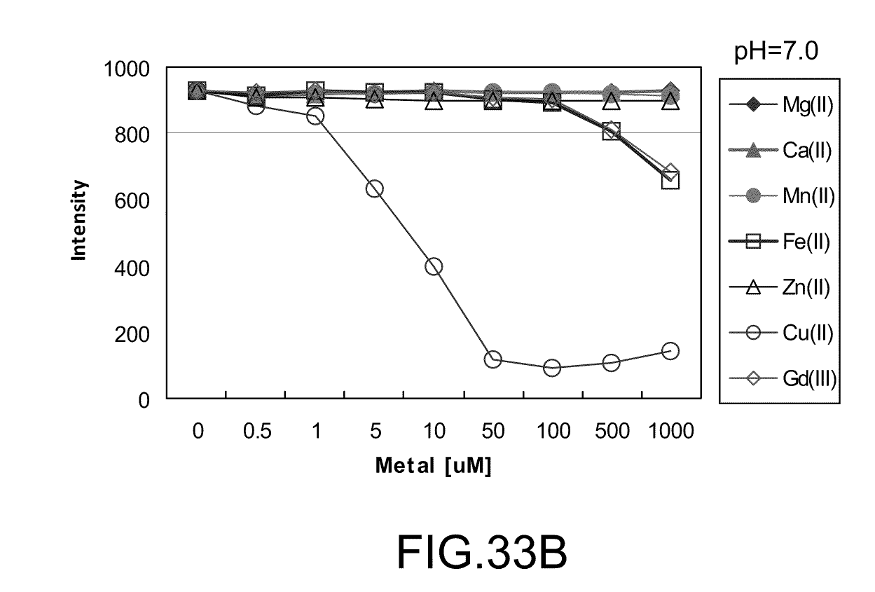

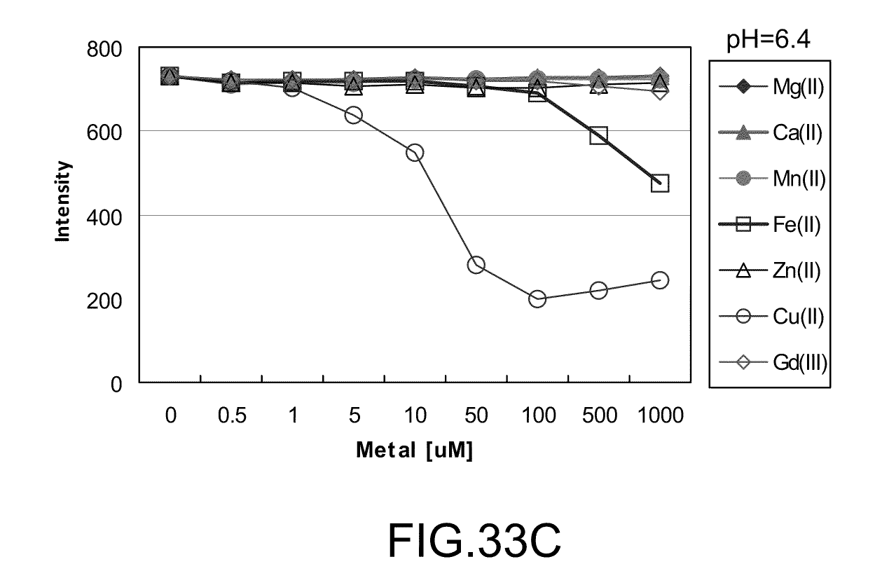

The electrospray mass spectrum of both P57 and the P57/Gd complex are shown in FIGS. 30A-30B. To better characterize the chemistry of P57, a high resolution 600 MHz ¹H NMR scan of the peptide in a 90/10% H₂O/D₂O sodium phosphate buffered (pH=7.6) solvent mixture was conducted. The ¹H NMR spectrum was obtained at room temperature, by averaging 512 individual NMR scans. The hydrophilic region of the spectrum is shown in FIG. 32.

Fluorescence titrations were used to determine the ability of P57/Gd to bind with various cations in aqueous solutions of 10 μM P57/Gd and 20 mM buffer. These solutions of P50 were titrated with CaCl₂, MgCl₂, MnCl₂, FeCl₂, CuSO₄, ZnCl₂, and GdCl₃ (FIGS. 32A-32C). The excitation filter wavelength was 485 nm, and the fluorescence emission filter was 520 nm. The solutions were buffered at pH=6.4, pH=7.0, and pH=7.6, using either NEM or MES, as appropriate. The fluorescence of each condition was tracked over a period of 120 minutes, to insure equilibrium was obtained. After 120 minutes the pH of each condition was rechecked with pH paper, to confirm the pH remained constant during the experiment.

P57/Gd had no fluorescence change following the treatment with large excesses of Ca⁺² and Mg⁺², indicting the peptide P57/Gd does not interact with these ions. Interactions with Zn⁺² and Fe⁺² were present, but were weak.

The reaction with P57/Gd with Cu⁺² was strong at all pH values studied. The selectivity of P57/Gd for Cu⁺² was significantly higher than P1P, P15, P20, and P41. The fluorescence intensity was also significantly larger, due to the lack of the dabcyl quenching group. The model presented in Equation 4 was found to describe the interaction of P57/Gd with Cu⁺² and yielded binding constants of 15.4 μM±7.2 μM, 2.7 μM±0.8 μM, and 6.3 μM±3.2 μM, at pH 6.4, 7.0, and 7.6, respectively.

The copper detection mechanism of P57/Gd is different from that of P15/Gd. P15/Gd was designed to exhibit a change in relaxivity during the binding of a targeted transition metal. P57/Gd was designed to exhibit a neutral relaxivity response to copper ions and instead operate on a more solely pharmacokinetic mechanism. The K(DO3A) group is located on the C-terminus of the P57/Gd peptide and is significantly separated from the copper binding site inside the core peptide region. This causes the solvent structure and molecular dynamics of the DO3A/Gd group to be invariant to copper binding. During copper binding P57/Gd however still does experience a change in its net charge. The positive increase in net charge alters the solubility and transportation kinetics of P57/Gd, thereby altering the pharmacokinetic profile of the smart contrast agent. The accumulation and clearance of the contrast agent can then be used to construct a digital map of localized copper concentrations. This situation is different than that of the previous P15/Gd example, where a second K(DO3A) group on the N-terminus is located much closer to the copper binding site. The inclusion of the dabycl group in the P15/Gd also enhances the structural changes that occur to P15/Gd during copper binding, as evidenced by the upfield shifts in dabcyl and FAM ¹H NMR resonances observed during the copper titration of P41.

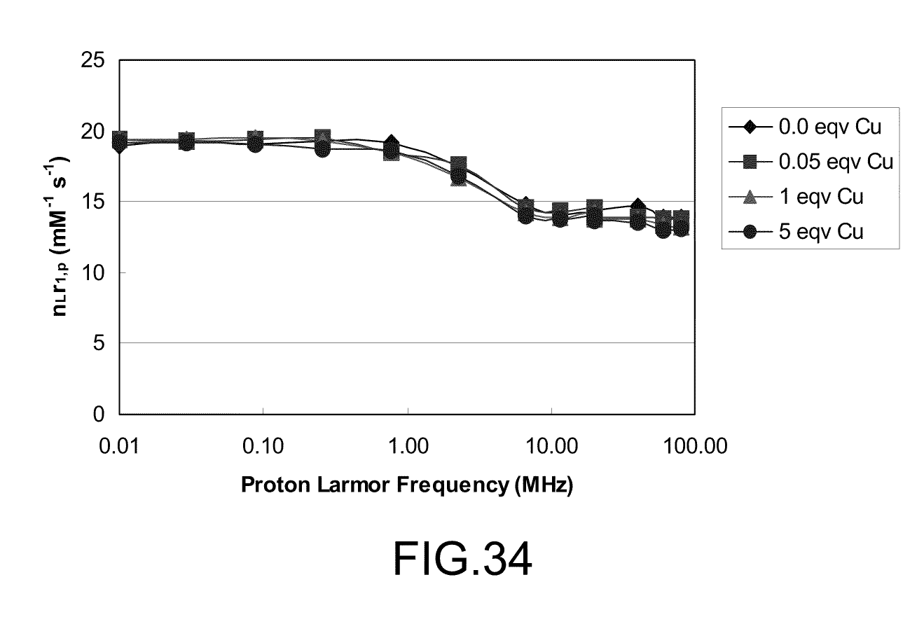

To determine the effect of copper ions on the T₁ relaxation times, 0.1 mM P57/Gd solutions in 50 mM phosphate buffer, at pH=7.0, were incubated with 0.0 mM, 0.05 mM, 0.1 mM, and 0.5 mM CuSO₄. The nuclear magnetic relaxation dispersion (NMRD) profiles are given in FIG. 34. The longitudinal relaxivity was measured at field strengths of from 0.01 MHz to 80 MHz. The average value of [symbol] n_{L}r_{1,p} [symbol] was found to be 19.0 mM⁻¹ s⁻¹ and 14.0 mM⁻¹ s⁻¹, at Larmor frequencies of 0.01 MHz and 80 MHz, respectively. The r₁ relaxivity of P57/Gd was invariant to increasing amounts of copper (II) ions. This property is advantageous, because it permits a simplified correlation between the ¹H MRI signal and the concentration to be obtained for each voxel. The invariance of the r₁ relaxivity P57/Gd to copper(II) ions also illustrates that a gadolinium-copper exchange is not a factor.

The T₁ relaxivity of P57/Gd was also measured using 7 Telsa MRI machine designed for rodent in vivo studies. Concentrations of 0 μM, 10 μM, 25 μM, 50 μM, and 100 μM of P57/Gd were buffered to near neutral conditions and imaged.

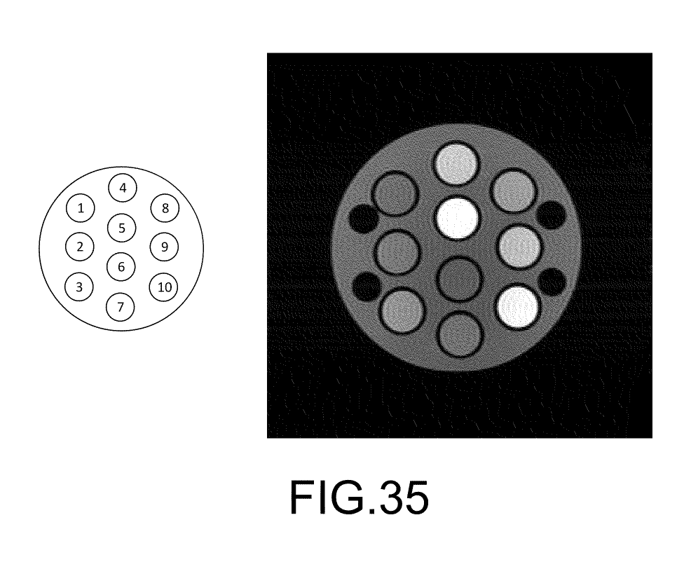

FIG. 35 illustrates the in vitro imaging of P57/Gd. Wells numbered 1-5 and 6-10 represent increasing concentrations of P57/Gd, ran in duplicate. The concentrations displayed are 0 μM, 10 μM, 25 μM, 50 μM, and 100 μM of P57/Gd. As shown in FIG. 35, the image progression from low to high concentrations was clearly visible. The decrease in T₁ was found to fit very well to equation 1 and the value of r_{1,p} was determined to between 6.7-6.0/(mM s). This relaxation rate was found to be superior to both that of Magnevist® and Dotarem®, measured using the same procedure to be 4.0/(mM s) and 2.7/(mM s) respectively. The relaxivity for P57/Gd is also larger than other smart ¹H MRI contrast agents currently reported for copper.

The already strong relaxivity of the P57/Gd peptide can be further increased in another exemplary embodiment, by substituting a large bulky hydrophobic amino acid for the glutamine residue in P57. Amino acid substitutions such as alanine, valine, leucine, and isoleucine hinder the rotation of the K(DO3A/Gd) group, increasing the relaxivity of the peptide. Chemical modifications to the alkyl side chain in the K(DO3A) group can also hinder the Gd site's rotation and increase the relaxivity. This can be accomplished in several ways, including the incorporation of a carbon double bond, a shorting of the alkyl chain length, or the use of branched alkyl chains, such as —CH₂—CH(CH₃)—CH₂—NH-DO3A or —CH₂—C(CH₃)₂—NH-DO3A.

The pharmacokinetics of P57/Gd in ICR mice was studied using a 200 μL tail vein injection of 2.0 mM P57/Gd in saline solution. No adverse toxicological reactions were observed in the mice following the injections. The mice were sacrificed at 5, 10, and 15 minutes post injection, and their serum and internal organs were harvested. The brain, kidney, and liver tissues were homogenized prior to analysis. The fluorescence intensity of P57/Gd is quite strong and was easily detected in all samples, using fluorescence RP-HPLC. The RP-HPLC conditions were as follows: solution A, 5 mM phosphate buffer (pH=7.6); solution B, 5 mM phosphate buffer (pH=7.6) and 65% acetonitrile; column, 4.6×250 mm Cosmosil 5C18-SR-II; detector settings, excitation wavelength=490 nm and emission wavelength=525 nm. The organ/blood ratio for P57/Gd was determined to be between 1.3%, 4.8%, and 23.7% for the brain, liver, and kidneys, respectively.

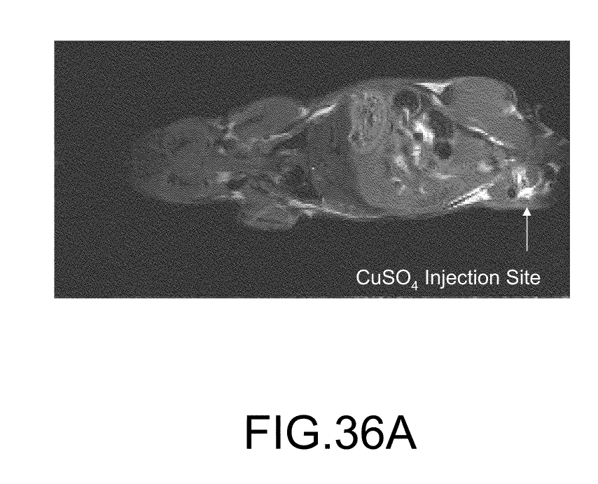

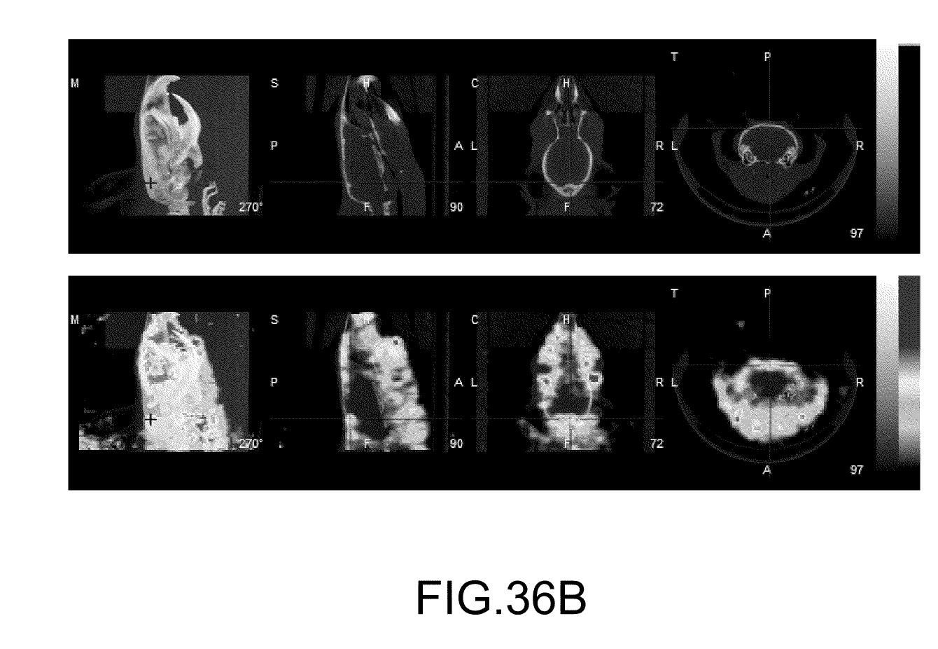

As shown in FIG. 36A, to confirm the ability of P57/Gd to detect copper in vivo using ¹H MRI scans, a male ICR mouse was injected in the leg muscle (inside the thigh) with a 100 μL solution of 1 mg/mL CuSO₄. The mouse was then administered a 200 μL tail vein injection of 2.0 mM P57/Gd in saline solution. After 15 minutes the mouse was imaged using an ¹H MRI scan. The image was obtained using a spin echo method with TR=500 ms, TE=14 ms, 10 slices, thickness=1.5 mm and the gap=0.5 mm. The injection site on the left, hind leg showed an increased positive signal. A similar feature in the control, right, hind leg was not visible.

In another example of the current invention, the Gd ion in P57/Gd can be replaced by radioactive metal ion, such as ¹¹¹In or ^{99m}Tc, to produce a SPECT contrast agent. In this implementation of the current invention, P57 is synthesized in the same manner as described above. The Gd complexation procedure however, is amended to use a radioactive salt solution instead of GdCl₃. This permits an additional detection channel using SPECT techniques, without significantly altering the chemical properties of the peptide. The substitution is an advantageous, because SPECT imaging can detect lower contrast agent concentrations than MRI.

As shown in FIG. 36B to illustrate this application, a low dose ˜5 μM solution of P57/¹¹¹In (426 μCi) was injected into the tail vein of an ICR mouse. Dynamic 9 minute brain SPECT scans were conducted over the first 20 minutes post injection. These scans were followed by a standard CT scan. The SPECT and CT scans were fused to produce a composite image. The top row of images are taken from only the CT scan and the bottom row represents the CT images with the SPECT intensity of the P57/¹¹¹In compound overlaid.

Localized regions of P57/¹¹¹In inside the head are clearly visible and the concentration of contrast agent can be quantified. The SPECT intensity was highest around the optical cavity and inside hind region of the upper skull (FIG. 36B). This area contains the cerebellum and pineal gland. These areas had a significantly elevated amount of P57/¹¹¹In as compared to other head and neck tissues. The pineal grand is known to posses high copper concentrations.

### Example 7

### A Highly Selective Agent for Copper that Uses Only a Single Fluorescent Labeling Group

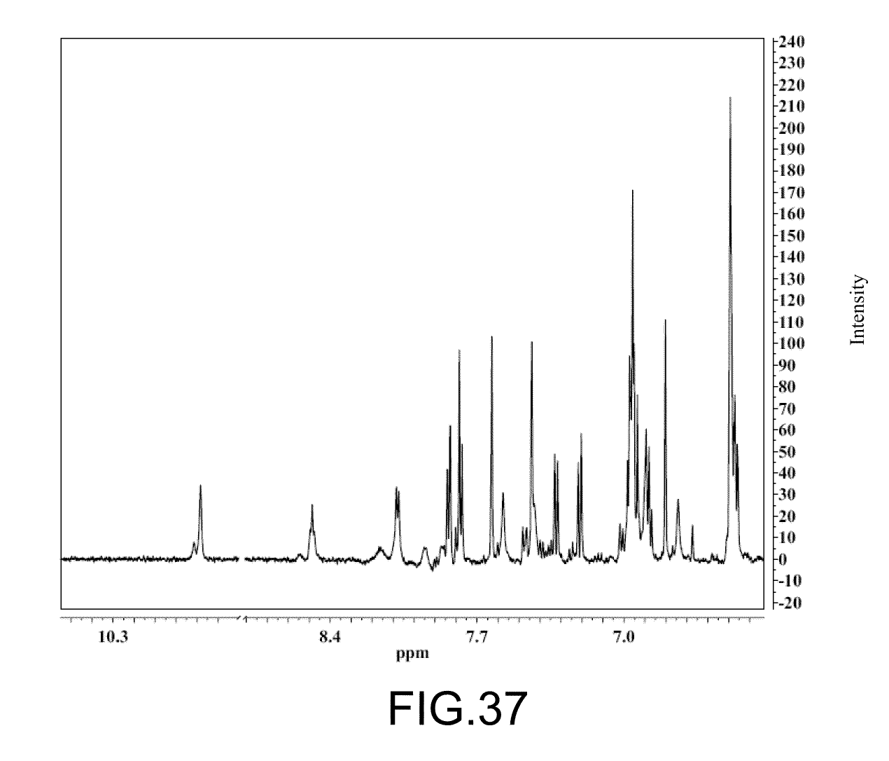

The compound used in this example will be referred to as P54 and includes the following sequence, Ac-K(6-FAM)PHGGGWGQP-NH₂. P54 was chemically synthesized, in its neutral free amine-acetylated form, using standard Fmoc/tBu methods (W C Chan and P D White 2000). Commercially available Fmoc-Lys(FAM)-OH building blocks were found to sometimes suffer from lower than advertised purity and a unclear isomeric chemical structure. To achieve a higher quality and more precise synthesis, Fmoc-Lys(FAM)-OH building blocks were not used. The 6-FAM group was instead directly added to the target lysine side chain, in an additional step following the primary synthesis of the peptide backbone chain. The crude peptide product was purified using reverse-phase HPLC. Purification was continued until >90% purity, as confirmed by HPLC and electrospray mass spectrometry (FIG. 37). To better characterize the chemistry of P54, a high resolution 600 MHz ¹H NMR scan of the peptide in a 90/10% H₂O/D₂O sodium phosphate buffer (pH=7.6) solvent mixture was conducted. The ¹H NMR spectrum was obtained at room temperature, by averaging 512 individual NMR scans. The hydrophilic region of the spectrum is shown in FIG. 37.

Fluorescence titrations were used to determine the ability of P54 to bind with various cations in aqueous solutions of 10 μM P54 and 20 mM NEM buffer (pH=7.6). These solutions of P50 were titrated with CaCl₂, ZnCl₂, and CuSO₄ (FIG. 37A). The excitation filter wavelength was 485 nm, and the fluorescence emission filter was 520 nm. The fluorescence of each condition was tracked over a period of 120 minutes, to insure equilibrium was obtained. After 120 minutes the pH of each condition was rechecked with pH paper, to confirm the pH remained constant during the experiment.

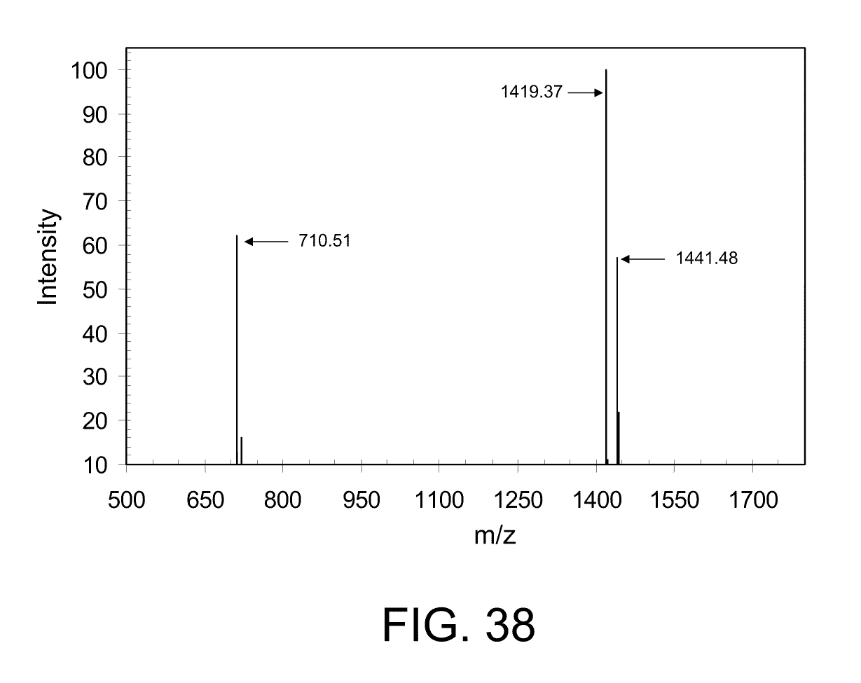

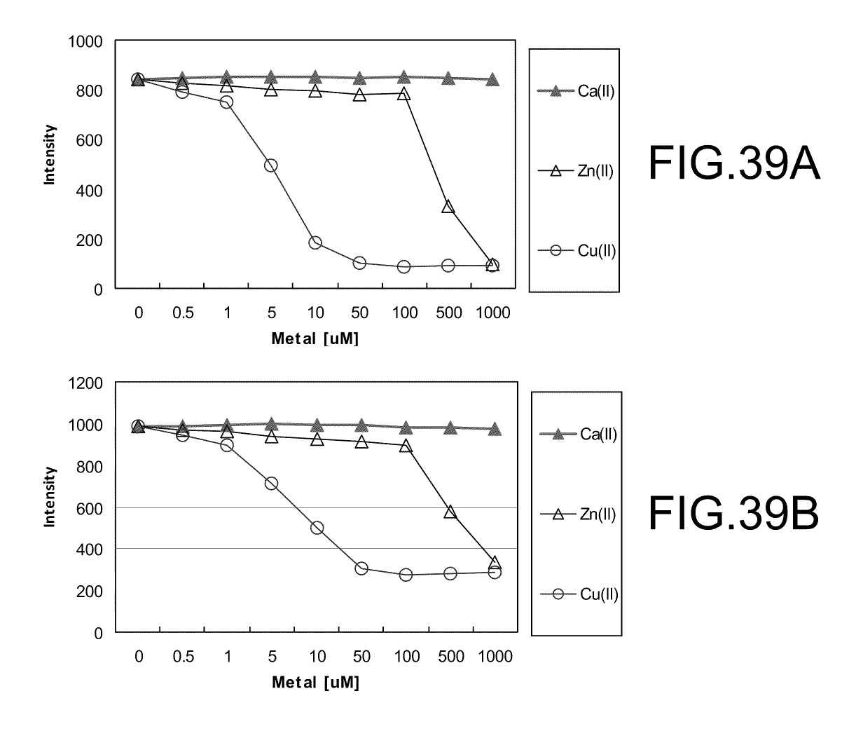

The reaction with P54 with Cu⁺² was strong. The model presented in Equation 4 was found to describe the interaction of P54 with Cu⁺² and yielded a binding constant of 0.26±0.48 μM. A weaker, but still strong, binding affinity of 1.75±1.0 μM with Cu⁺² was found for the P58 peptide, which has the K(6-FAM) residue located near the C-terminus, on the other end of the amino acid chain (FIG. 38B).

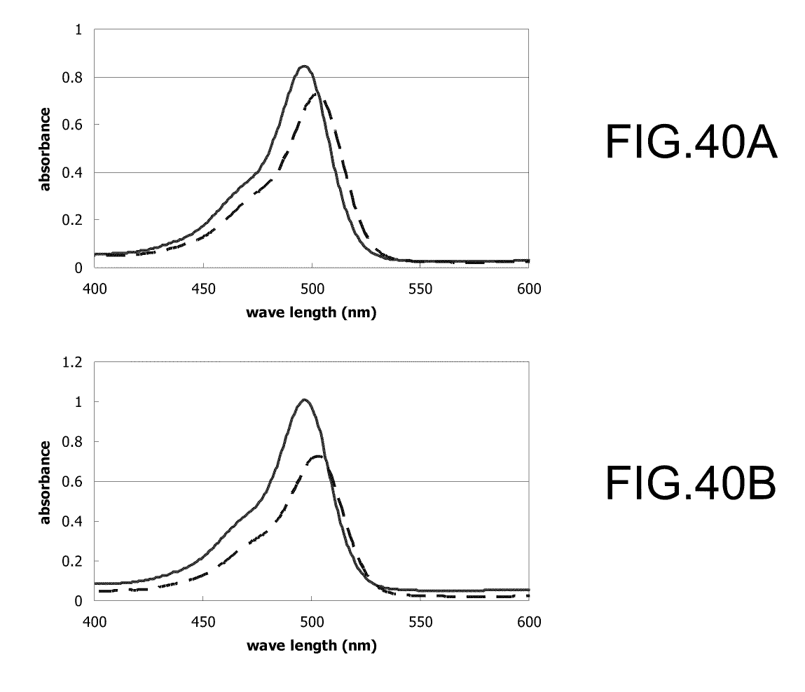

FIG. 40A-40B illustrate the absorbance spectrum of P54 and P58, respectively and where the solid curve denotes the contrast agent only and the dashed curve denotes the contrast agent in the presence of 3 equivalents of copper(II) ions at pH=7.6. The reporting mechanism of P54 and P58 for copper is the result of two concurrent electrostatic effects: an ion-dipole interaction and a dipole-dipole interaction.

The ion-dipole interaction is caused by the interaction of the electrostatic field of the copper ion with the conjugated π bond system of the 6-FAM group. The electrostatic field of the copper ion splits the quantum energy levels of the electrons in the 6-FAM group. As shown in FIGS. 40A-40B, this reduces the energy gap between the ground and excited states and results in a red shifting of the absorbance spectrums during copper binding. The red shift reduces the amount of energy absorbed from the 485 nm excitation beam and alters the fluorescence intensity recorded at 520 nm.

The second dipole-dipole interaction effect is between the FAM group and the copper/core peptide complex. Copper/histidine complexes have weak absorbance maximums (molar extinction coefficients ˜80 cm⁻¹ M⁻¹) around 550-700 nm. Even though this absorbance is much weaker than other acceptor groups, the Forester distance is still estimated to be on the same order as the peptide chain length. In the absence of copper, no acceptor group exists, and no energy transfer occurs. Once copper binds the histidine residue, the newly formed copper/core peptide complex acts as the acceptor group, and energy can be transferred via a FRET mechanism. In P54, the 6-FAM group is very close to the copper complex site and shows a strong decrease in fluorescence intensity during copper binding. In P58, the 6-FAM group is spatially located farther away from the copper complex site and shows a weaker decrease in fluorescence.

The P54 and P58 exhibited higher fluorescence intensities, as compared to the P1P and P41 implementations, because the baseline fluorescence intensity is considerably higher, without the presence of the dabcyl quenching group.

Interaction of P54 with Zn⁺² ions was several orders of magnitude weaker than with copper. The weak binding of zinc ions for P54, P58, P57/Gd, and P50 illustrates that the presence of the dabcyl group is an important factor for the detection of zinc ions. Zinc forms a complex with both the dabcyl labeling group and the core peptide region. Zinc ions are generally silent and to do not have a significant absorbance overlap with the 6-FAM fluorescence spectrum. The detection phenomenon for zinc ions by P1P, P41, and P20, therefore, is primarily driven by an interaction between the FAM and dabcyl groups. The overall result is that P54 is a selective contrast agent for copper ions and does not report on zinc ion concentrations.

Although a few exemplary embodiments of the present invention have been shown and described, it would be appreciated by those skilled in the art that changes may be made in these exemplary embodiments, without departing from the principles and spirit of the invention, the scope of which is defined in the claims and their equivalents.

1. A smart contrast agent comprising:
a core peptide comprising a transition metal binding domain having at least 70% homology to a fragment of a prion extended octarepeat region, the extended octarepeat region selected from SEQ ID NO: 3; and
a labeling group attached to the N-terminus of the core peptide.

2. The smart contrast agent of claim 1, wherein the core peptide comprises from one to two copies of a peptide having at least 70% homology to a sequence selected from SEQ ID NOS: 4-22.

3. The smart contrast agent of claim 1, wherein the end termini of the smart contrast agent are in a zwitterionic form.

4. The smart contrast agent of claim 1, wherein the smart contrast agent has an acetylated N-terminus, a neutral amine C-terminus, or a combination thereof.

5. The smart contrast agent of claim 1, wherein the core peptide forms a complex with copper that acts as a FRET acceptor.

6. The smart contrast agent of claim 1, wherein the fluorescence spectrum and/or the absorbance of the labeling group changes, when a transition metal is bound to the smart contrast agent.

7. The smart contrast agent of claim 1, wherein the N-terminus labeling group increases the resistance of the core peptide to chemical degradation by blood enzymes.

8. The smart contrast agent of claim 1, wherein the N-terminus labeling group comprises a 6-carboxyfluorescein (6-FAM) group, a 5-carboxyfluorescein (5-FAM) group, or a 1,4,7,10-tetraazacyclododecane-1,4,7-trisacetic acid (DO3A) group.

9. The smart contrast agent of claim 1, wherein the N-terminus labeling group is chemically attached to the side chain of a lysine amino acid, and the lysine residue is attached to the N-terminus of the core peptide.

10. The smart contrast agent of claim 1, wherein the N-terminus labeling group comprises a lysine residue and a 6-carboxyfluorescein (6-FAM) group attached to the side chain of the lysine residue.

11. The smart contrast agent of claim 1, wherein the N-terminus labeling group comprises a lysine residue attached to a D-proline N-terminus residue of the core peptide and a 5-carboxyfluorescein (5-FAM) group attached to the side chain of the lysine residue.

12. The smart contrast agent of claim 1, wherein the N-terminus labeling group comprises a lysine residue attached to the N-terminus of the core peptide and a 5-carboxyfluorescein (5-FAM) group attached to the N-terminus of the lysine residue.

13. The smart contrast agent of claim 1, further comprising a labeling group that is attached to the C-terminus of the core peptide.

14. The smart contrast agent of claim 13, wherein the C-terminus labeling group comprises a dabcyl group attached to the C-terminus of the core peptide.

15. The smart contrast agent of claim 13, wherein the C-terminus labeling comprises a 1,4,7,10-tetraazacyclododecane-1,4,7-trisacetic acid (DO3A) group, a diethylenetriamine-N,N,N′,N″,N″-pentaacetic acid (DTPA) group, a 10-(2-hydroxypropyl)-1,4,7,10-tetraazacyclododecane-1,4,7-triacetic acid (HP-DO3A) group, a 1,4,7,10-tetraazacyclododecane-1,4,7,10-tetraacetic acid (DOTA) group, a diethylenetriamine pentaacetic acid bismethylamide (DTPA-BMA) group, a metallofullerene group, or a texaphyrin group.

16. The smart contrast agent of claim 15, wherein the C-terminus labeling group is bound to gadolinium, europium, dysprosium, indium, or technetium.

17. The smart contrast agent of claim 13, wherein the C-terminus labeling group comprises a gold nanoparticle, a gold nanoshell, or an iron oxide particle.

18. The smart contrast agent of claim 13, wherein the C-terminus labeling group comprises a trifluoromethyl group, a (2S,4S)-4-fluoro-pyrrolidine group, a (2S,4R)-4-fluoro-pyrrolidine group, a 4-fluoro-phenylalanine group, a fluorophenyl group, or a fluorodeoxyglucose (FDG) group.

19. The smart contrast agent of claim 13, wherein the C-terminus labeling group comprises a radioactive ¹⁸F isotope.

20. The smart contrast agent of claim 13, wherein the C-terminus labeling group is chemically attached to the side chain of a lysine amino acid, and the lysine residue is attached to the C-terminus of the core peptide.

21. A method of treating disease, comprising:
delivering the smart contrast agent of claim 1 into a mammal; and
altering the distribution of the transition metal inside the mammal, such that negative effects of a disease are reduced.

22. The method of claim 21, wherein the smart contrast agent of claim 1 reduces an excess of transition metal in the mammal.

23. The method of claim 21, wherein the smart contrast agent is complexed with a transition metal, prior to being applied.

24. The method of claim 23, wherein the transition metal is delivered across the blood-brain barrier of the mammal and is then released.

25. The method of claim 21 where the transition metal is copper.

26. The method of claim 21, wherein the disease is one selected from the group consisting of Alzheimer's disease, Parkinson's disease, Lewy body disease, Wilson's disease, Menkes disease, bipolar disorders, schizophrenia, depression, and cancer.


SEQUENCE LISTING (22 sequences)

SEQ ID NO: 1 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 3 - protein, 50 aa, Artificial Sequence
  Description of Artificial Sequence Extended octarepeat region
PQGGGGWGQPHGGGWGQPHGGGWGQPHGGGWGQPHGGGWGQGGGTHSQWN

SEQ ID NO: 4 - protein, 8 aa, Artificial Sequence
  Description of Artificial Sequence Synthetic probe
PHGGGWGQ

SEQ ID NO: 5 - protein, 8 aa, Artificial Sequence
  Description of Artificial Sequence Synthetic probe
PHGGGWGA

SEQ ID NO: 6 - protein, 14 aa, Artificial Sequence
  Description of Artificial Sequence Synthetic probe
PHGGGWGQGGGTHS

SEQ ID NO: 7 - protein, 8 aa, Artificial Sequence
  Description of Artificial Sequence Synthetic probe
PHGGSWGQ

SEQ ID NO: 8 - protein, 16 aa, Artificial Sequence
  Description of Artificial Sequence Synthetic probe
PHGGGWGQPHGGSWGQ

SEQ ID NO: 9 - protein, 16 aa, Artificial Sequence
  Description of Artificial Sequence Synthetic probe
PHGGSWGQPHGGGWGQ

SEQ ID NO: 10 - protein, 8 aa, Artificial Sequence
  Description of Artificial Sequence Synthetic probe
PHGGGWGL

SEQ ID NO: 11 - protein, 8 aa, Artificial Sequence
  Description of Artificial Sequence Synthetic probe
PHGGGWGV

SEQ ID NO: 12 - protein, 8 aa, Artificial Sequence
  Description of Artificial Sequence Synthetic probe
PHGGGWGI

SEQ ID NO: 13 - protein, 8 aa, Artificial Sequence
  Description of Artificial Sequence Synthetic probe
PHGGGWGF

SEQ ID NO: 14 - protein, 7 aa, Artificial Sequence
  Description of Artificial Sequence Synthetic probe
PHGGGWG

SEQ ID NO: 15 - protein, 9 aa, Artificial Sequence
  Description of Artificial Sequence Synthetic probe
PHGGGWGQP

SEQ ID NO: 16 - protein, 9 aa, Artificial Sequence
  Description of Artificial Sequence Synthetic probe
PHGGGWGAP

SEQ ID NO: 17 - protein, 9 aa, Artificial Sequence
  Description of Artificial Sequence Synthetic probe
PHGGGWGLP

SEQ ID NO: 18 - protein, 9 aa, Artificial Sequence
  Description of Artificial Sequence Synthetic probe
PHGGGWGVP

SEQ ID NO: 19 - protein, 9 aa, Artificial Sequence
  Description of Artificial Sequence Synthetic probe
PHGGGWGIP

SEQ ID NO: 20 - protein, 9 aa, Artificial Sequence
  Description of Artificial Sequence Synthetic probe
PHGGGWGFP

SEQ ID NO: 21 - protein, 8 aa, Artificial Sequence
  Description of Artificial Sequence Synthetic probe
PHGGGWGP

SEQ ID NO: 22 - protein, 7 aa, Artificial Sequence
  Description of Artificial Sequence Synthetic probe
PHGGGWP